(19) United States
(12) Patent Application Publication
(10) Pub. No.: US 2025/0332277 A1
(43) Pub. Date: Oct. 30, 2025

(54) PROTEIN TYROSINE KINASE 7 ANTIBODIES AND ANTIBODY-DRUG CONJUGATES

(71) Applicant: Eli Lilly and Company, Indianapolis, IN (US)

(72) Inventors: Ahmad Hossin AKHGAR, Gaithersburg, MD (US); Elaine Margaret CONNER, San Diego, CA (US); Kevin Charles LINDQUIST, Berkeley, CA (US); Divya SAGAR, Hoboken, NJ (US); David John STOKELL, Indianapolis, IN (US)

(21) Appl. No.: 19/189,334

(22) Filed: Apr. 25, 2025

Related U.S. Application Data
(60) Provisional application No. 63/638,968, filed on Apr. 26, 2024.

(51) Int. Cl. A61K 47/68; A61P 35/00; C07K 16/40

(52) U.S. Cl. CPC A61K 47/6871; A61K 47/68037; A61P 35/00; C07K 16/40; C12Y 207/10002; C07K 2317/33; C07K 2317/732; C07K 2317/734; C07K 2317/92

(57) ABSTRACT

The present disclosure provides PTK-7 antibody drug conjugates and pharmaceutical compositions thereof, and methods of using for the treatment of cancer.

### REFERENCE TO A SEQUENCE LISTING

The present application is being filed along with a Sequence Listing in ST.26 XML format. The Sequence Listing is provided as a file titled “30976_US” created 30 Jan. 2025 and is 63 kilobytes in size. The Sequence Listing information in the ST.26 XML format is incorporated herein by reference in its entirety.

### FIELD

The disclosure relates to the field of medicine. More particularly, the disclosure relates to Protein Tyrosine Kinase 7 (PTK-7) antibodies, antibody-drug conjugates, and pharmaceutical compositions thereof, and their use in treating cancer.

### BACKGROUND

PTK-7 belongs to the family of Wnt-related pseudokinases and is overexpressed in multiple tumor types, including, triple-negative breast, non-small-cell lung, colorectal, gastric, esophageal, and ovarian cancers.

Antibody-drug conjugates (ADCs) for use as oncology treatments contain a tumor-targeting antibody conjugated to a payload designed to be cell-killing once inside the tumor cell. Certain PTK-7 antibodies have been used to create ADCs with an MMAE payload (WO2015168019) and with a camptothecin analog (Kong et al. Mol Cancer Ther 22 (10): 1128).

ADCs for use in oncology are very challenging compounds to design since multiple aspects of the molecule must be balanced, including, sufficient specificity for the tumor target over healthy cells, acceptable toxicity while maintaining desirable activity against bystander tumor cells, and labile payloads to allow intracellular delivery yet maintain good physical and chemical stability.

### SUMMARY

There remains a need for PTK-7 ADCs for treating cancer. In particular, PTK-7 ADCs are needed with a sufficient therapeutic index based on better tolerability and/or better efficacy to support high enough doses to effectively kill the tumor cells but also be tolerable for the patients. In particular, a need remains for PTK-7 ADCs with an effector null antibody, fully human antibody, and a topoisomerase I payload. In particular, a need remains for PTK-7 ADCs with enhanced bystander activity against PTK-7-low tumors. In particular, a need remains for PTK-7 ADCs with enhanced bystander activity against heterogenous expressing PTK-7 tumors. In particular, a need remains for PTK-7 ADCs that have low immunogenicity, stable in vivo pharmacokinetics, and adequate chemical and physical stability. Additionally, a need remains for PTK-7 ADCs that possess one or more of the following features: exhibit better anti-tumor activity as measured in certain tumor models, enhanced bystander activity for PTK-7-low tumors, lower immunogenicity, no measurable antibody effector function, no Fc gamma receptor engagement, and/or better physical and chemical stability. The ADCs provided herein address one or more of these needs.

Provided herein are certain PTK-7 ADCs and compositions comprising a PTK-7 ADC. Also provided herein are methods of using the PTK-7 ADCs or compositions comprising a PTK-7 ADC for cancer in a subject.

In one aspect, provided herein is an antibody that binds human PTK-7, wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein:
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19;
c) the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29; or
d) the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

In a further aspect, provided herein is an antibody that binds human PTK-7, wherein the antibody comprises a heavy chain variable region (VH) and a light chain variable region (VL), wherein:
a) the VH comprises SEQ ID NO: 10 and the VL comprises SEQ ID NO: 11;
b) the VH comprises SEQ ID NO: 20 and the VL comprises SEQ ID NO: 21;
c) the VH comprises SEQ ID NO: 30 and the VL comprises SEQ ID NO: 31; or
d) the VH comprises SEQ ID NO: 39 and the VL comprises SEQ ID NO: 40.

In another aspect, provided herein is an antibody that binds human PTK-7, wherein the antibody comprises a heavy chain (HC) and a light chain (LC), wherein:
a) the HC comprises amino acids 2-441 of SEQ ID NO: 2 and the LC comprises SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23;
d) the HC comprises amino acids 2-444 of SEQ ID NO: 32 and the LC comprises amino acids 2-215 of SEQ ID NO: 33.

In another aspect, provided herein is an antibody-drug conjugate (ADC) comprising a PTK-7 antibody disclosed herein conjugated directly or through a linker to a cytotoxic agent.

In a further aspect, provided herein is an ADC wherein the cytotoxic agent is a camptothecin analog comprising Formula I:

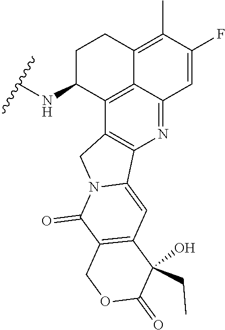 [SMILES: *N[C@H]1CCc2c(C)c(F)cc3nc4c(c1c23)Cn1c-4cc2c(c1=O)COC(=O)[C@]2(O)CC]

In another aspect, provided herein is an ADC of Formula II:

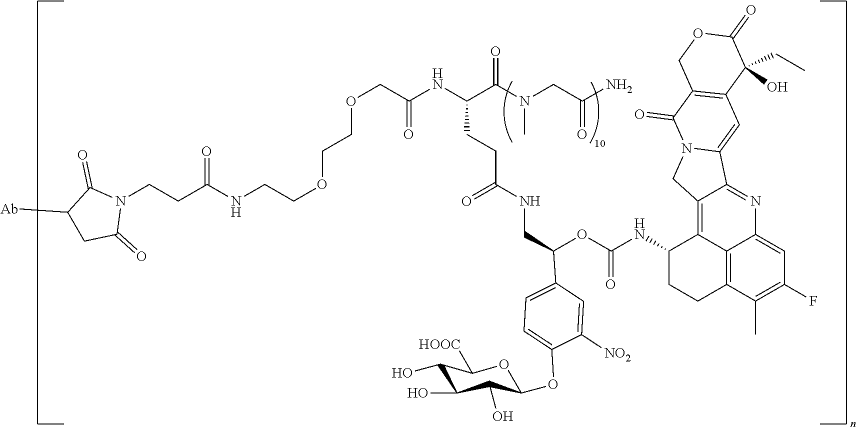

wherein: Ab is a PTK-7 antibody disclosed herein, and n is from about 1 to about 16.

In another aspect, provided herein is an ADC of Formula III:

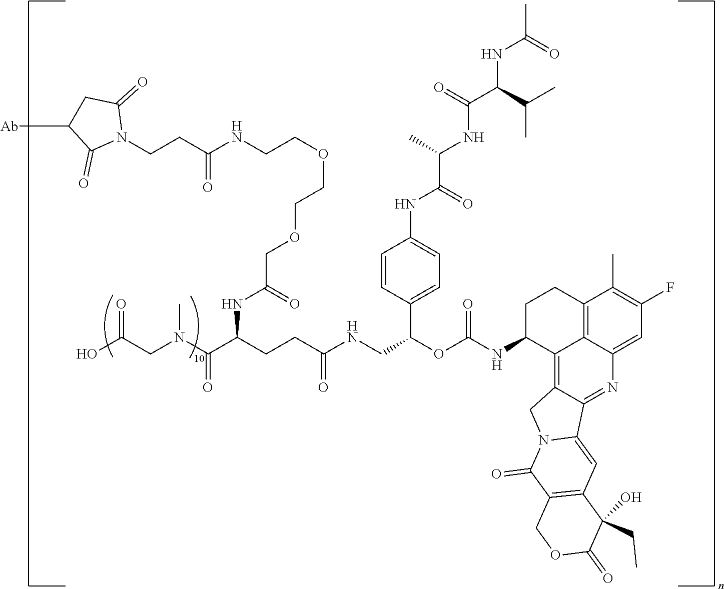

wherein: Ab is a PTK-7 antibody disclosed herein, and n is from about 1 to about 16.

In another aspect, provided herein is a pharmaceutical composition comprising a PTK-7 antibody disclosed herein, and one or more pharmaceutically acceptable carriers, diluents, or excipients. In another aspect, provided herein is a pharmaceutical composition comprising a PTK-7 ADC disclosed herein, and one or more pharmaceutically acceptable carriers, diluents, or excipients.

In another aspect, provided herein is a method of treating cancer, comprising administering to a patient in need thereof, an effective amount of a PTK-7 ADC disclosed herein. In a further aspect, provided herein is a method of treating cancer, comprising administering to a patient in need thereof, an effective amount of a PTK-7 ADC disclosed herein, wherein the cancer is ovarian cancer, lung cancer, breast cancer, gastric cancer, kidney cancer, prostate cancer, liver cancer, or colorectal cancer.

### DETAILED DESCRIPTION

### PTK-7

As used herein, “human PTK-7” refers to human protein tyrosine kinase 7, also known as colon carcinoma kinase-4 (CCK-4). An amino acid sequence of human PTK-7 can be found at NP_002812, including the signal peptide, as provided in SEQ ID NO: 1.

### PTK-7 Antibodies (Also Known as Anti-PTK-7 Antibodies)

The term “antibody,” as used herein, refers to an immunoglobulin molecule that binds an antigen. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). The CDRs of the present disclosure are determined by North.

Certain antibodies described herein contain an IgG1 Fc region or an Fc region derived from human IgG1, e.g., a modified IgG1 Fc region having altered Fc effector functions. IgG1 is known to induce antibody-dependent cell cytotoxicity (ADCC) and complement-dependent cytotoxicity (CDC). Some antibodies of the present disclosure have amino acid substitutions introduced into IgG1 Fc region altering effector function. According to some disclosed herein, mutations are introduced in the Fc region at positions 234 and 235 (according to the EU Index numbering). According to some disclosed herein, mutations are introduced in the Fc region at positions 234, 235, and 265 (according to the EU Index numbering). In some aspects, the PTK-7 antibodies of the present disclosure comprise a modified human IgG1 Fc region comprising alanine at residues 234 and 235, and serine at position 265 (according to the EU Index numbering, also called hIgG1 effector null or hIgG1EN Fc region). In further aspects, some antibodies have further mutations in the Fc region, including glutamine, alanine, or glycine at position 297, alanine or glutamine at position 322, alanine or glycine at position 329, and/or alanine or serine at position 331 (according to the EU Index numbering). In some aspects, these antibody mutations are alanine at position 234, glutamic acid at position 235, alanine at position 237, serine at position 330, and serine at position 331 (according to the EU Index numbering). In further aspects, these amino acid substitutions introduced into IgG1 Fc region reduced or eliminated measurable antibody effector function.

In certain aspects of the present disclosure, the PTK-7 antibody has a modified human IgG1 or human IgG4 constant domain comprising one or more engineered cysteine residues. In further aspects, the antibody comprises an engineered cysteine in one or more sites within the heavy chain constant domain 1 (CH1), the heavy chain constant domain 2 (CH2), and/or the heavy chain constant domain 3 (CH3).

Mammalian expression of antibodies typically results in glycosylation. Glycosylation of antibodies is typically either N-linked or O-linked. N-linked glycosylation refers to the attachment of a carbohydrate moiety to the side chain of an asparagine residue. O-linked glycosylation refers to the attachment of a sugar, for example N-acetylgalactosamine, galactose, or xylose to a hydroxyamino acid. Typically, glycosylation occurs in the Fc region of the antibody at a highly conserved N-glycosylation site (e.g., position 297 in IgG1, according to IMGT or EU Index numbering). Glycosylation sites can be modified to alter glycosylation (e.g., blocking or reducing glycosylation or altering the amino acid sequence to produce additional or diverse glycosylation).

Mammalian expression of antibodies from IgG subclasses can result in clipping of C-terminal amino acids from one or both heavy chains; for example, one or two C-terminal amino acids can be removed for IgG1 antibodies. For IgG1 antibodies, if a C-terminal lysine is present, then it may be truncated or clipped off from the heavy chain during expression. Additionally, a penultimate glycine may also be truncated or clipped off from the heavy chain as well.

Mammalian expression of antibodies can also result in the modification of N-terminal amino acids. For example, where the N-terminal most amino acid of a heavy chain or light chain is a glutamine or glutamic acid, it may be modified into pyro-glutamic acid. For example, where the C-terminal most amino acid of a heavy chain or light chain is a lysine or glycine, it may be removed.

The terms “nucleic acid” or “polynucleotide”, as used interchangeably herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

Polynucleotides of the present disclosure may be expressed in a host cell, for example after the polynucleotides have been operably linked to an expression control sequence. Expression control sequences capable of expression of polynucleotides to which they are operably linked are well known in the art. For example, an expression vector may include a sequence that encodes one or more signal peptides that facilitate secretion of the polypeptide(s) from a host cell. The signal peptide may be an immunoglobulin signal peptide or a heterologous signal peptide, for example. Expression vectors containing a polynucleotide of interest (e.g., a polynucleotide encoding a polypeptide of an antibody) may be transferred into a host cell by well-known methods. Additionally, expression vectors may contain one or more selection markers, e.g., tetracycline, neomycin, and dihydrofolate reductase, to aid in detection of host cells transformed with the desired polynucleotide sequences.

A host cell includes cells stably or transiently transfected, transformed, transduced or infected with one or more expression vectors expressing all or a portion of an antibody of the present disclosure. According to some aspects, a host cell may be stably or transiently transfected, transformed, transduced, or infected with an expression vector expressing HC polypeptides and an expression vector expressing LC polypeptides of an antibody of the present disclosure. In some aspects, a host cell may be stably or transiently transfected, transformed, transduced, or infected with an expression vector expressing HC and LC polypeptides of an antibody of the present disclosure. The antibody of the present disclosure may be produced in mammalian cells such as CHO, NS0, HEK293 or COS cells according to techniques well known in the art.

Medium, into which an antibody of the present disclosure has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

In an aspect, provided herein is an antibody that binds human PTK-7, wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9. In another aspect, provided herein is an antibody that binds human PTK-7, wherein the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19. In another aspect, provided herein is an antibody that binds human PTK-7, wherein the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29. In another aspect, provided herein is an antibody that binds human PTK-7, wherein the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

In an aspect, provided herein is an antibody that binds human PTK-7, wherein the antibody comprises a heavy chain variable region (VH) and a light chain variable region (VL), wherein the antibody comprises a VH comprising SEQ ID NO: 10 and a VL comprising SEQ ID NO: 11. In another aspect, provided herein is an antibody that binds human PTK-7, wherein the antibody comprises a VH comprising SEQ ID NO: 20 and a VL comprising SEQ ID NO: 21. In another aspect, provided herein is an antibody that binds human PTK-7, wherein the antibody comprises a VH comprising SEQ ID NO: 30 and a VL comprising SEQ ID NO: 31. In another aspect, provided herein is an antibody that binds human PTK-7, wherein the antibody comprises a VH comprising SEQ ID NO: 39 and a VL comprising SEQ ID NO: 40.

In a further aspect, provided herein is an antibody that binds human PTK-7, wherein the antibody comprises a heavy chain variable region (VH) and a light chain variable region (VL), and wherein the antibody has a human IgG1 or IgG4 isotype. In a further aspect, wherein the antibody has a human IgG1 isotype. In a further aspect, wherein the antibody comprises alanine at residues 234 and 235 (according to EU Index numbering). In a further aspect, wherein the antibody further comprises serine at position 265 (according to EU Index numbering). In another aspect, wherein the antibody has a human IgG4 isotype.

In an aspect, provided herein is an antibody that binds human PTK-7, wherein the antibody comprises a heavy chain (HC) and a light chain (LC), wherein:
a) the HC comprises amino acids 2-441 of SEQ ID NO: 2 and the LC consists of SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23;
d) the HC comprises amino acids 2-444 of SEQ ID NO: 32 and the LC comprises amino acids 2-215 of SEQ ID NO: 33.

In an aspect, provided herein is an antibody that binds human PTK-7, wherein the antibody comprises a heavy chain (HC) and a light chain (LC), wherein the HC comprises amino acids 2-441 of SEQ ID NO: 2 and the LC comprises SEQ ID NO: 3. In an aspect, provided herein is an antibody that binds human PTK-7, wherein the antibody comprises a heavy chain (HC) and a light chain (LC), wherein the HC comprises amino acids 2-448 of SEQ ID NO: 12 and the LC comprises amino acids 2-215 of SEQ ID NO: 13. In an aspect, provided herein is an antibody that binds human PTK-7, wherein the antibody comprises a heavy chain (HC) and a light chain (LC), wherein the HC comprises amino acids 2-447 of SEQ ID NO: 22 and the LC comprises amino acids 2-215 of SEQ ID NO: 23. In an aspect, provided herein is an antibody that binds human PTK-7, wherein the antibody comprises a heavy chain (HC) and a light chain (LC), wherein the HC comprises amino acids 2-444 of SEQ ID NO: 32 and the LC comprises amino acids 2-215 of SEQ ID NO: 33.

In a further aspect, provided herein is an antibody that binds human PTK-7, wherein the HC consists of SEQ ID NO: 2 and the LC consists of SEQ ID NO: 3. In another aspect, provided herein is an antibody that binds human PTK-7, wherein the HC consists of SEQ ID NO: 12 and the LC consists of SEQ ID NO: 13. In another aspect, provided herein is an antibody that binds human PTK-7, wherein the HC consists of SEQ ID NO: 22 and the LC consists of SEQ ID NO: 23. In another aspect, provided herein is an antibody that binds human PTK-7, wherein the HC consists of SEQ ID NO: 32 and the LC consists of SEQ ID NO: 33.

In another aspect, provided herein are different mammalian cells comprising a DNA molecule comprising a polynucleotide sequence encoding polypeptides having the amino acid sequences of SEQ ID NO: 2 and SEQ ID NO: 3, SEQ ID NO: 12 and SEQ ID NO: 13, SEQ ID NO: 22 and SEQ ID NO: 23, SEQ ID NO: 32 and SEQ ID NO: 33, wherein the cell is capable of expressing PTK-7 antibodies disclosed herein.

In another aspect, provided herein are mammalian cells comprising a first DNA molecule and a second DNA molecule, wherein the first DNA molecule comprises a polynucleotide sequence encoding polypeptides having the amino acid sequence as follows, and wherein the second DNA molecule comprises a polynucleotide sequence encoding polypeptides having the amino acid sequence as follows, wherein the cell is capable of expressing PTK-7 antibodies disclosed herein:

| first DNA encodes polypeptide | second DNA encodes polypeptide |
|---|---|
| SEQ ID NO: 2 | SEQ ID NO: 3, |
| SEQ ID NO: 12 | SEQ ID NO: 13, |
| SEQ ID NO: 22 | SEQ ID NO: 23, |
| SEQ ID NO: 32 | SEQ ID NO: 33. |

In another aspect, provided herein is a process for producing a PTK-7 antibody comprising cultivating one of the mammalian cells disclosed herein under conditions such that the antibody is expressed, and recovering the expressed antibody.

In another aspect, provided herein is an antibody produced by cultivating a mammalian cell comprising a DNA molecule comprising a polynucleotide sequence encoding polypeptides having the amino acid sequences of SEQ ID NO: 2 and SEQ ID NO: 3, SEQ ID NO: 12 and SEQ ID NO: 13, SEQ ID NO: 22 and SEQ ID NO: 23, SEQ ID NO: 32 and SEQ ID NO: 33, under conditions such that the antibody is expressed, and recovering the expressed antibody.

In another aspect, provided herein is an antibody produced by cultivating a mammalian cell comprising a first DNA molecule and a second DNA molecule, wherein the first DNA molecule comprises a polynucleotide sequence encoding polypeptides having the amino acid sequence following, and wherein the second DNA molecule comprises a polynucleotide sequence encoding polypeptides having the amino acid sequence following under conditions such that the antibody is expressed, and recovering the expressed antibody:

| first DNA encodes polypeptide | second DNA encodes polypeptide |
|---|---|
| SEQ ID NO: 2 | SEQ ID NO: 3, |
| SEQ ID NO: 12 | SEQ ID NO: 13, |
| SEQ ID NO: 22 | SEQ ID NO: 23, |
| SEQ ID NO: 32 | SEQ ID NO: 33. |

The term “cofetuzumab” as used herein refers to a humanized anti-PTK-7 IgG1 antibody (hu6M024) with the sequence as disclosed in WO2012112943, expressed and purified using standard conditions. The term “cofetuzumab pelidotin” refers to an ADC with a cleavable valine-citrulline linker, auristatin-0101 as the payload with a drug-to-antibody ratio (DAR) of 4.

### Payloads

PTK-7 antibodies of the present disclosure can be conjugated to various payloads (including pharmaceutically acceptable salts thereof) to form an antibody drug conjugate (ADC). Suitable moieties for conjugation to the PTK-7 antibodies disclosed herein include cytotoxic agents (e.g., chemotherapeutic agents), prodrug converting enzymes, radioactive isotopes or compounds, toxins, and other known payloads in the art.

Exemplary ADCs herein utilize camptothecin-based payloads (e.g., a camptothecin analog). Camptothecin analogs are topoisomerase I (TOPO 1) inhibitors that have been shown to have anticancer activity. Camptothecin and its analogs bind to the TOPO 1/DNA complex which prevents reannealing leading to cell death from the accumulation of partially cleaved DNA. Other camptothecin analogs known in the art can be used as payloads, such as topotecan, irinotecan, SN-38, belotecan, exatecan, deruxtecan (Dxd), and their salts, such as exatecan mesylate.

In an aspect, provided herein is an ADC, wherein the camptothecin analog is exatecan. In an aspect, provided herein is an ADC, wherein the exatecan comprises one of Formula IV, V, or VI, respectively:

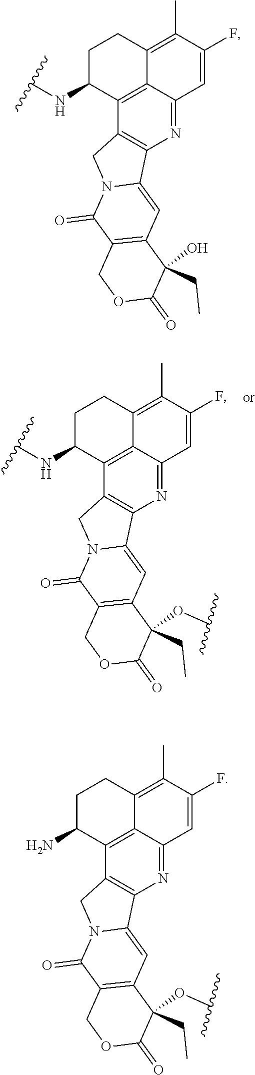

Other payloads for ADCs known in the art, such as maytansinoids (e.g., DM1 and DM4), pyrrolobenzodiazepines (e.g., PBD dimer), auristatin peptides (e.g., MMAE and MMAF), duocarmycins, calicheamicins, DNA minor groove binders (e.g., enediynes and lexitropsins), and taxanes (e.g., paclitaxel and docetaxel).

### Self-Immolative Units

Self-immolation, or self-removal, of a part of the ADC can be designed into the global structure of the ADC. Self-immolation typically involves a trigger group being activated and leading to spontaneous chemical and/or biological reactions which cause the elimination of the group itself, the self-immolation unit. The self-immolative unit can provide positive attributes to the ADC, such as providing space to reduce steric hindrance of cellular proteases reaching the peptide cleavage site in the ADC.

In some aspects of the present disclosure, the ADC described herein contains a self-immolative unit. When present, the self-immolative unit on the ADC is exposed and triggered after the ADC linker is cleaved, such as the peptide unit by a cellular protease or as the sugar cleavable unit by a cellular enzyme. In further aspects, the self-immolative unit is a para-aminobenzyloxycarbonyl (PABC), ortho-aminobenzyl carbonate (OABC), or another self-immolative unit known in the art.

In some aspects of the present disclosure, the ADC described herein comprises a self-immolative unit of Formula VII:

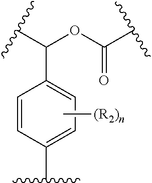

In some aspects of the present disclosure, the ADC described herein comprises a self-immolative unit of Formula VIII:

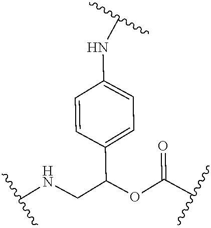

In some aspects of the present disclosure, the ADC described herein does not contain a self-immolative unit. In these aspects, the camptothecin analog described herein is directly linked to the peptide unit or sugar cleavable unit.

It is understood that payloads described herein may exist as stereoisomers. Embodiments of the present disclosure include all enantiomers, diastereomers, and mixtures thereof.

### Drug-to-Antibody Ratio (DAR)

In the present disclosure, the average drug load when referring to a composition comprising a population of ADCs is the drug-to-antibody ratio or DAR. The average number of drugs per antibody in a preparation may be characterized by conventional means such as mass spectroscopy, HIC, ELISA, and HPLC. Higher DAR can produce more potent ADCs, but also, higher DAR can also result in destabilization, aggregation, increased off-target toxicity, and enhanced drug clearance from systemic circulation.

In some aspects, the DAR is from 1 to about 16, about 2 to about 14, or about 2 to about 10. In an aspect, the DAR is from about 2 to about 5. In a further aspect, the DAR is an average of 4. In another aspect, the DAR is from about 6 to about 10. In a further aspect, the DAR is an average of 8.

In some aspects, at least about 50%, 60%, 70%, 80%, 90%, 95%, 96%, 97%, 98%, or 99% of the ADC disclosed herein has a DAR of 4. In some aspects, at least about 50%, 60%, 70%, 80%, 90%, 95%, 96%, 97%, 98%, or 99% of the ADC disclosed herein has a DAR of 8. In some aspects, the ADC disclosed herein is present in a composition wherein the ADC present in the composition has an average DAR greater than about 1, 2, 3, 4, 5, 6, 7, or 8.

### Linkers

As disclosed herein, payloads can be conjugated with a PTK-7 antibody to form a PTK-7 ADC described herein by methods understood by one of skill in the art. One example of such conjugation would include connection of a payload described herein to a PTK-7 antibody described herein via a linker.

Linkers used for ADCs are designed for stability in plasma to allow time for the ADC to localize to the target cells. Releasing the payload too soon lowers the therapeutic index of the ADC by damaging non-targeted tissue of all kinds. When the ADC is internalized into the target cell, then the linker should provide a mechanism for liberation of the payload such so the payload can work as designed.

Linkers known to those of skill in the art contain, for example, cleavable moieties and noncleavable moieties. Accordingly, provided herein are ADCs where the payload, such as a camptothecin analog, is conjugated to an antibody via a linker with a cleavable moiety or is conjugated to an antibody via a linker with a non-cleavable moiety.

Any suitable linkers known in the art can be used in preparing the ADCs of the present disclosure. In certain aspects, the linkers comprise reactive groups capable of conjugating with both the antibodies of the present disclosure and the drug or cytotoxic agent. Examples include but are not limited to N-succinimidyl-4-(N-maleimidomethyl)-cyclohexane-1-carboxylate (SMCC), N-succinimidyl-4-(iodoacetyl)-aminobenzoate (SIAB), bis-maleimidopolyethyleneglycol (BMPEO), BM (PEO) 2, BM (PEO) 3, N-(b-maleimidopropyloxy) succinimide ester (BMPS), g-maleimidobutyric acid N-succinimidyl ester (GMBS), e-maleimidocaproic acid N-hydroxysuccinimide ester (EMCS), 5-maleimidovaleric acid NHS ester, N-succinimidyl-4-(N-maleimidomethyl)-cyclohexane-1-carboxy-(6-amidocaproate), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido) propionate (SBAP), N-succinimidyl iodoacetate (SIA), k-maleimidoundecanoic acid N-succinimidyl ester (KMUA), N-succinimidyl 4-(p-maleimidophenyl)-butyrate (SMPB), succinimidyl-6-(b-maleimidopropionamido) hexanoate (SMPH), succinimidyl-(4-vinylsulfonyl)benzoate (SVSB), dithiobis-maleimidoethane (DTME), 1,4-bis-maleimidobutane (BMB), 1,4-bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(y-maleimidobutryloxy) sulfosuccinimide ester (sulfo-GMBS or sGMBS), N-(e-maleimidocaproyloxy) sulfosuccimido ester (sulfo-EMCS), N-(K-maleimidoundecanoyloxy) sulfosuccinimide ester (sulfo-KMUS), and sulfosuccinimidyl 4-(p-maleimidophenyl) butyrate (sulfo-SMPB), succinimidyl 6-hydrazinonicotinamide acetone hydrazone (SANH), succinimidyl 4-hydrazidoterephthalate hydrochloride (SHTH), succinimidyl hydrazinium nicotinate hydrochloride (SHNH), succinimidyl-p-formyl benzoate (SFB), and succinimidyl-p-formylphenoxyacetate (SFPA), V-succinimidyl-3-(2-pyridyldithio) propionate (SPDP), V-succinimidyl-4-(2-pyridyldithio) pentanoate (SPP), N-succinimidyl-4-(2-pyridyldithio) butanoate (SPDB), and V-succinimidyl-4-(2-pyridyldithio) 2-sulfo butanoate (sulfo-SPDB).

In certain aspects of the present disclosure, the ADC comprises a cleavable linker. Known in the art are different mechanisms to employ in the linker to release the drug or cytotoxic agent. Included among these mechanisms are (1) utilizing protease cleavage or other enzymatic cleavage sites in the linker that will be cleaved by cellular enzymes (e.g. cathepsin B or beta-glucuronidase), (2) using the lower pH of the lysosome to cause hydrolysis of an acid-labile unit in the linker, or (3) utilizing the higher intracellular levels of glutathione to reduce a disulfide bridge in the linker.

In some aspects of the present disclosure, the ADC described herein comprises a linker which comprises a peptide unit which provides a site for protease cleavage. In some aspects, the peptide unit is -Gly-Gly-Gly-, -Ala-Val-, -Val-Ala-, -Val-Cit-, -Val-Lys-, -Lys-Val-, -Phe-Lys-, -Lys-Phe-, -Lys-Lys-, -Ala-Lys-, -Lys-Ala-, -Phe-Cit-, -Cit-Phe, -Leu-Cit-, -Cit-Leu-, -Ile-Cit-, -Phe-Ala-, -Ala- Phe-, -Phe-Phe-Lys-, -Lys-Phe-Phe-, -Gly-Phe-Lys-, -Lys-Phe-Gly-, -Leu-Ala-Leu-, -Ile-Ala-Leu-, -Leu-Ala-Ile-, -Val-Ala-Val-, -Ala-Leu-Ala-Leu-(SEQ ID NO: 49), -Leu-Ala-Leu-Ala-(SEQ ID NO: 50), -Gly-Phe-Leu-Gly-(SEQ ID NO: 51), -Gly-Leu-Phe-Gly-(SEQ ID NO: 52), -Val-Arg-, -Arg-Val-, -Arg-Arg-, -Ala-Ala-, -Ala-Met-, -Met-Ala-, -Thr-Thr-, -Thr-Met-, -Met-Thr-, -Leu-Ala-, -Ala-Leu-, -Cit-Val-, -Gln-Val-, -Val-Gln-, -Ser-Val-, -Val- Ser-, -Ser-Ala-, -Ser-Gly-, -Ala-Ser-, -Gly-Ser-, -Leu-Gln-, -Gln-Leu-, -Phe-Arg-, -Arg-Phe-, -Tyr-Arg-, -Arg-Tyr-, -Phe-Gln-, -Gln-Phe-, -Val-Thr-, -Thr-Val-, -Met-Tyr-, -Tyr-Met-, -Ala-Ala-, -Ala-Ala-Ala-, -Ala-Ala-Ala-Ala-(SEQ ID NO: 53), -Gly-Ala-Gly-Gly-(SEQ ID NO: 54), -Gly-Gly-Ala-Gly-(SEQ ID NO: 55), -Gly-Val-Gly-Gly-(SEQ ID NO: 56), -Gly-Gly-Val-Gly-(SEQ ID NO: 57), -Gly-Phe-Gly-Gly-(SEQ ID NO: 58), or -Gly-Gly-Phe-Gly-(SEQ ID NO: 59). Citrulline is represented by Cit.

In aspects of the disclosure, the peptide unit contains all natural amino acids of the L-amino acid form. In further aspects, the peptide unit may comprise all D-amino acids or L-amino acids, or a combination therein. Accordingly, the peptide unit may comprise such as D-Val-D-Ala, L-Val-L-Ala, D-Val-L-Ala, or L-Val-D-Ala.

The term “sugar cleavable unit” as used herein refers a site in a linker containing a sugar moiety that can be cleaved by an enzyme, such as a lysosomal acid hydrolase like beta-glucuronidase. In some aspects of the present disclosure, the ADC provided herein comprises a linker which comprises a sugar cleavable unit which provides a site for cleavage. In further aspects, the sugar cleavable unit is a glucuronide or a galactoside, such as a beta-glucuronide or a beta-galactoside moiety. In further aspects the sugar cleavable unit comprises a beta-glucuronide of Formula IX:

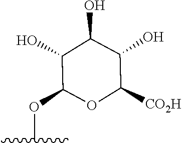 [SMILES: *O[C@@H]1O[C@H](C(=O)O)[C@@H](O)[C@H](O)[C@H]1O]

The term “hydrophobicity masking group” as used herein refers to a group that can reduce the apparent hydrophobicity of a compound. In some aspects of the present disclosure, the ADC described herein, particularly the linkers described herein, contains a hydrophobicity masking entity of polysarcosine, polyethylene glycol, or chitooligosaccharides. In further aspects, the hydrophobicity masking group, including polysarcosine, polyethylene glycol, or chitooligosaccharides, is in a branched configuration on the linker, rather than linearly connected. Examples of such a branched configuration for the hydrophobicity masking group are found in Formulas XXVI and XXI. The number of ethylene glycol or sarcosine moieties may vary in a wide range. For instance, the number of ethylene glycol or sarcosine moieties in the hydrophobicity masking entity may be between 2 and 500, between 5 and 100, or between 5 and 25. In further aspects, the ADC provided herein has a hydrophobicity masking entity of polysarcosine comprising from 2 to 50 sarcosine moieties, from 4 to 30 sarcosine moieties, from 6 to 24 sarcosine moieties, or from 10 to 12 sarcosine moieties. The number of chitosan in the chitooligosaccharide may vary between 2 and 20, or between 2 and 8.

In aspects of the present disclosure, the ADC comprises a linker which comprises a connecting unit that connects the linker-payload to the antibody. In further aspects, the ADC provided herein have a connecting unit that connects the cysteine(s) of the antibodies disclosed herein to the linkers and/or payloads described herein. Some of the chemistries used in the art to connect to the cysteine include maleimide, succinimide, or bromoacetamide chemistries, and can be utilized for ADCs of the present disclosure. In further aspects, spacers, such as chains of CH2 and/or PEG (such as 2-PEG or 3-PEG) are utilized as part of the connecting unit. In further aspects of the present disclosure, maleimide-type connecting units such as maleimidocaproyl (mc) or maleimidomethyl cyclohexane-1-carboxylate are used.

In aspects of the present disclosure, the maleimide-containing connecting unit which is conjugated to the antibody via Michael addition through a cysteine thiol, creating a succinimide linkage to form the ADC, can exist or slowly convert in vivo to a ring opened form by hydrolytic cleavage of the succinimide.

In some aspects of the present disclosure, provided herein is an ADC with a connecting unit of Formula X:

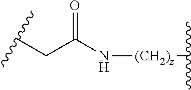

wherein z is from 1-5.

In some aspects of the present disclosure, provided herein is an ADC with a connecting unit of Formula XI:

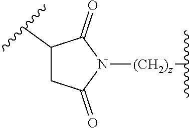

wherein z is from 1-5.

In some aspects of the present disclosure, provided herein is an ADC with a connecting unit of Formula XII:

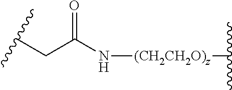

wherein z is from 1-5.

In some aspects of the present disclosure, provided herein is an ADC with a connecting unit of Formula XII:

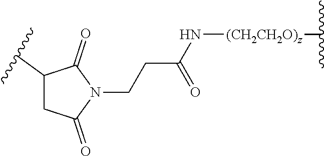

wherein z is from 1-5.

In another aspect, provided herein is an ADC, wherein the ADC is of Formula II, IIa, or IIb:

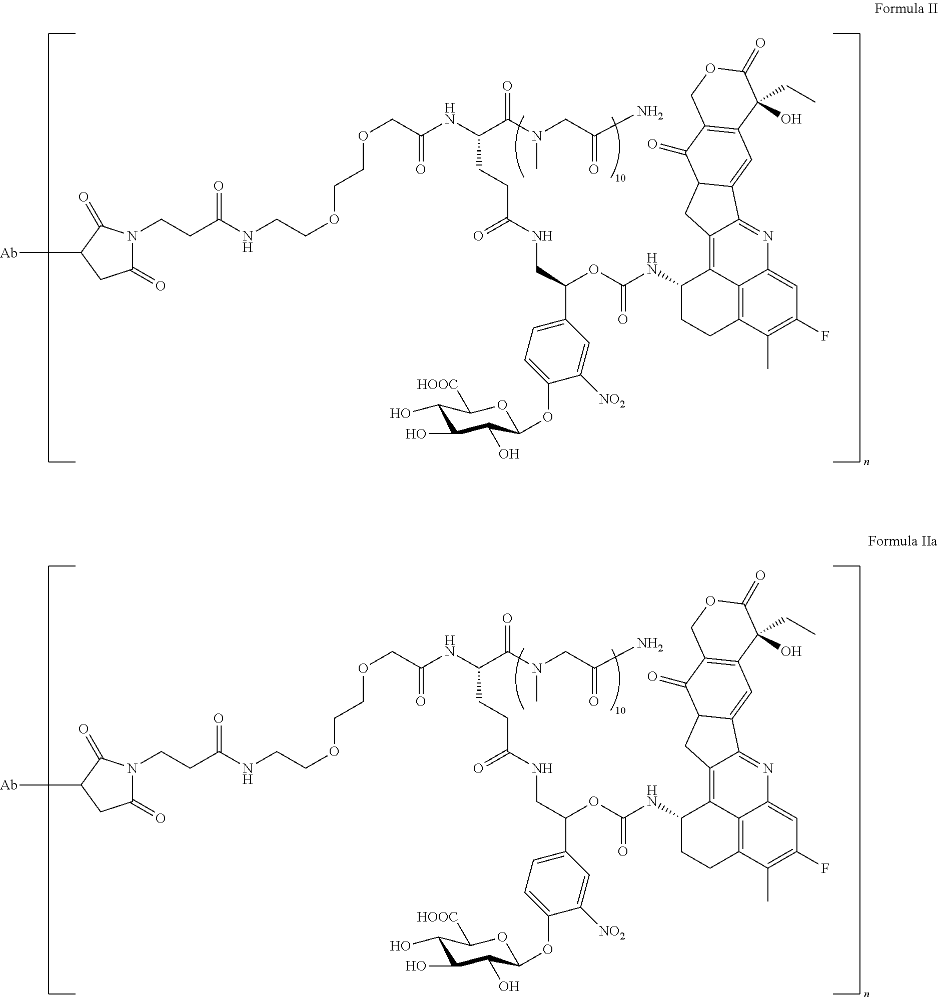

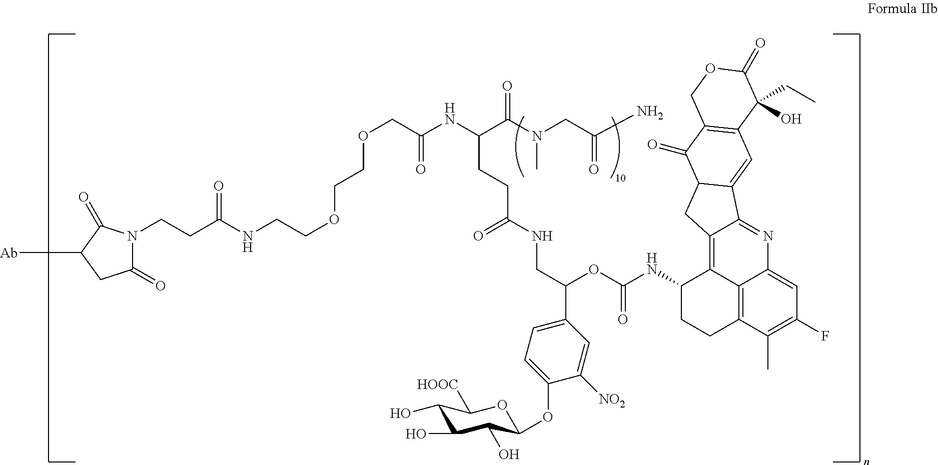

wherein: Ab is a PTK-7 antibody disclosed herein, and n is from about 1 to about 16.

In another aspect, provided herein is an ADC, wherein the ADC is of Formula III, IIIa, or IIIb:

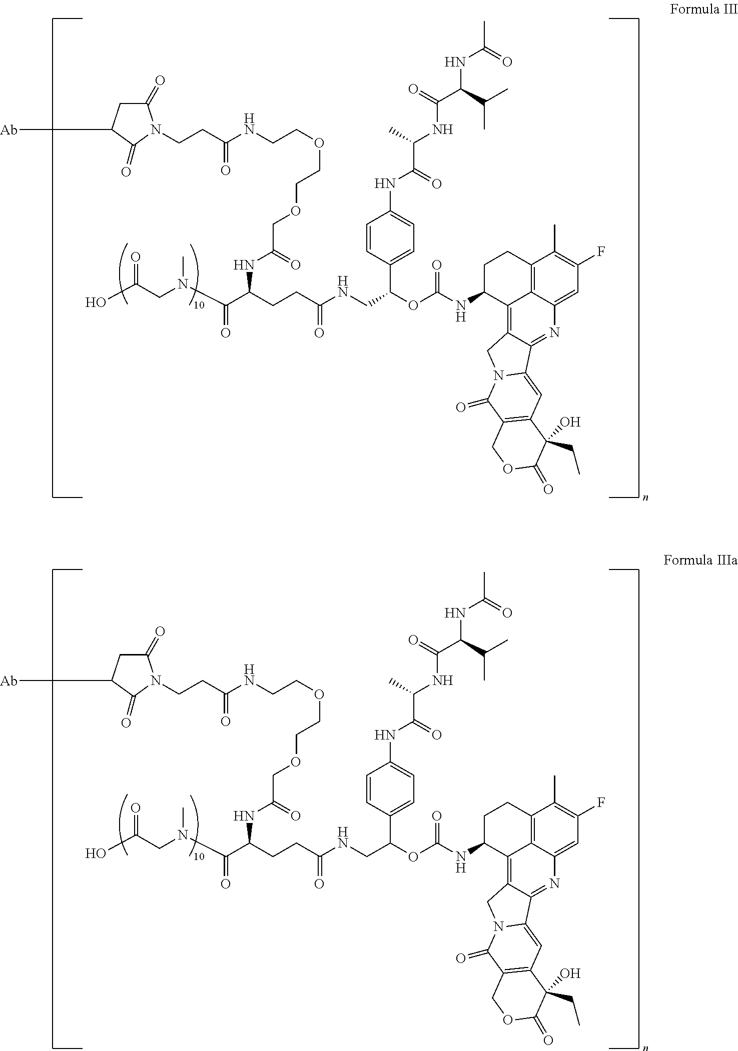

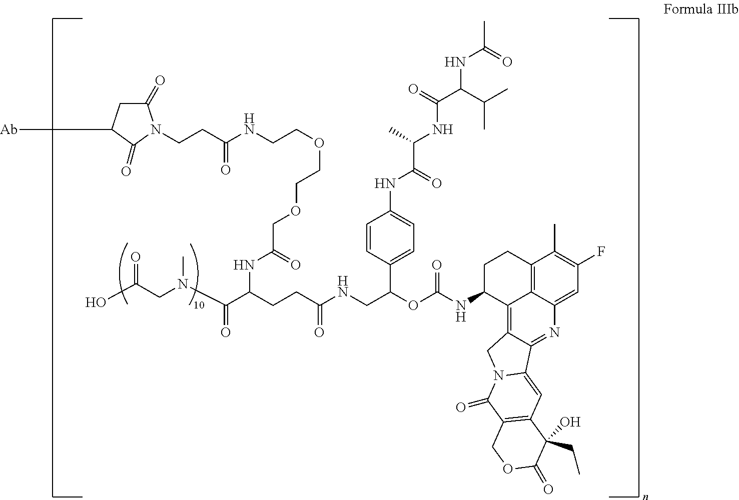

wherein: Ab is a PTK-7 antibody disclosed herein, and n is from about 1 to about 16.

In a further aspect, n is from about 2 to about 12. In another aspect, n is from about 2 to about 8. In another aspect, n is from about 4 to about 8. In another aspect, n is from about 8 to about 12. In another aspect, n is about 2. In another aspect, n is about 4. In another aspect, n is about 6. In another aspect, n is about 8. In another aspect, n is about 10. In another aspect, n is about 12.

In an aspect, disclosed herein, wherein the Ab comprises a HC comprising amino acids 2-441 of SEQ ID NO: 2 and a LC comprising SEQ ID NO: 3, and wherein n is about 8. In an aspect, disclosed herein, wherein the Ab comprises a HC comprising amino acids 2-441 of SEQ ID NO: 2 and a LC comprising SEQ ID NO: 3, and wherein n is about 4. In a further aspect, disclosed herein, wherein the Ab comprises a HC consisting of SEQ ID NO: 2 and a LC consisting of SEQ ID NO: 3.

In an aspect, disclosed herein, wherein the Ab comprises a HC comprising amino acids 2-448 of SEQ ID NO: 12 and a LC comprising amino acids 2-215 of SEQ ID NO: 13, and wherein n is about 8. In an aspect, disclosed herein, wherein the Ab comprises a HC comprising amino acids 2-448 of SEQ ID NO: 12 and a LC comprising amino acids 2-215 of SEQ ID NO: 13, and wherein n is about 4. In a further aspect, disclosed herein, wherein the Ab comprises a HC consisting of SEQ ID NO: 12 and a LC consisting of SEQ ID NO: 13.

In an aspect, disclosed herein, wherein the Ab comprises a HC comprising amino acids 2-447 of SEQ ID NO: 22 and a LC comprising amino acids 2-215 of SEQ ID NO: 23, and wherein n is about 8. In an aspect, disclosed herein, wherein the Ab comprises a HC comprising amino acids 2-447 of SEQ ID NO: 22 and a LC comprising amino acids 2-215 of SEQ ID NO: 23, and wherein n is about 4. In a further aspect, disclosed herein, wherein the Ab comprises a HC consisting of SEQ ID NO: 22 and a LC consisting of SEQ ID NO: 23.

In an aspect, disclosed herein, wherein the Ab comprises a HC comprising amino acids 2-444 of SEQ ID NO: 32 and a LC comprising amino acids 2-215 of SEQ ID NO: 33, and wherein n is about 8. In an aspect, disclosed herein, wherein the Ab comprises a HC comprising amino acids 2-444 of SEQ ID NO: 32 and a LC comprising amino acids 2-215 of SEQ ID NO: 33, and wherein n is about 4. In a further aspect, disclosed herein, wherein the Ab comprises a HC consisting of SEQ ID NO: 32 and a LC consisting of SEQ ID NO: 33.

It is understood that linkers and linker-payloads described herein may exist as stereoisomers. Embodiments of the present disclosure include all enantiomers, diastereomers, and mixtures thereof.

### Conjugation to Anti-PTK-7 Antibody

Methods to conjugate antibodies disclosed herein to the payloads and linker-payloads disclosed herein are known in the art. In some methods, the antibody is conjugated to a linker in a first reaction, and then the antibody and linker are conjugated to the payload in a second reaction. In some methods, the antibody is conjugated to the payload or payload/linker in one reaction.

In some aspects of the present disclosure, a PTK-7 antibody described herein is covalently linked to a camptothecin analog described herein through the thiol group of one or more cysteine residues located on the PTK-7 antibody. In further aspects, the cysteine residue(s) used for conjugation are each an interchain disulfide cysteine residue. Known in the art are methods to control reduction of the interchain disulfides to allow for conjugation to those involved cysteines. In other aspects, the cysteine residue or residues used for conjugation are engineered into the antibody, separate from the ones used for interchain disulfides.

In some aspects of the ADC described herein, the Ab is conjugated to the linker-payload via the thiol group of one or more cysteine residues. In some aspects, the Ab is conjugated via the thiol group of one or more cysteine residues in the HC of the Ab disclosed herein. In some aspects, the Ab is conjugated via the thiol group of one or more cysteine residues and wherein the cysteines are selected from C219, C225, and C228 of the heavy chain and C215 of the light chain, or combinations thereof.

In other aspects of the present disclosure, a PTK-7 antibody described herein is covalently linked to a camptothecin analog described herein through the amino group of one or more lysine residues located on the PTK-7 antibody. In other aspects of the present disclosure, a PTK-7 antibody described herein is covalently linked to a camptothecin analog described herein through the amino group of one or more glutamine residues located on the PTK-7 antibody.

In an aspect, provided herein is an ADC, wherein connection of the cytotoxic agent, cytotoxic agent-self-immolative spacer, or cytotoxic agent-self-immolative spacer-linker to the antibody occurs through a thiol group on one or more cysteines of the antibody. In a further aspect, wherein the one or more cysteines are each a natural cysteine in the hinge region of the antibody.

In an aspect, provided herein is an ADC, wherein the linker-payload used for conjugation to the PTK7 antibodies described herein is of Formula XIV or XV:

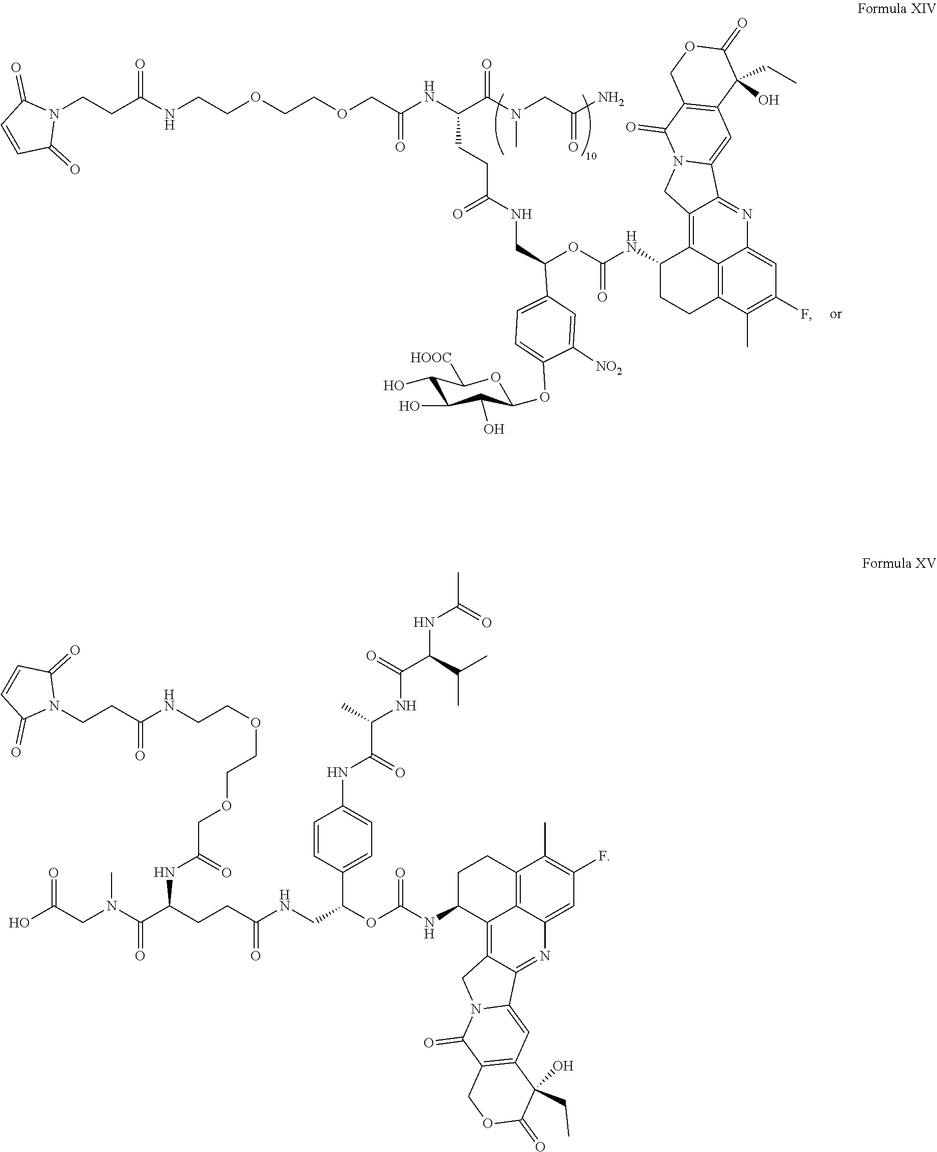

The present disclosure provides a method of producing an ADC, the method comprising the steps of:
(a) reducing a PTK-7 antibody disclosed herein with a reducing agent to produce a reduced PTK-7 antibody; and
(b) contacting the reduced PTK-7 antibody with a compound of the present disclosure to produce the conjugate, wherein the compound comprises a linker-payload disclosed herein, such as Formula XIV or XV. A further aspect, wherein the reducing agent is DTT or TCEP.

The conjugates of the present disclosure, or salts thereof, may be readily prepared by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates of the disclosure, or salts thereof. The product of each step can be recovered by conventional methods well known in the art, including extraction, evaporation, precipitation, chromatography, filtration, trituration, and crystallization. All substituents unless otherwise indicated, are as previously defined. The reagents and starting materials are readily available to one of ordinary skill in the art. The following preparations, examples, and assays further illustrate the disclosure, but should not be construed to limit the scope of the disclosure in any way.

### Therapeutic Applications

In another aspect, provided herein are methods of treating cancer, comprising administering to a patient in need thereof, an effective amount of a PTK-7 ADC or pharmaceutical composition described herein. In a further aspect, provided herein is a method of treating cancer, comprising administering to a patient in need thereof, an effective amount of an ADC or pharmaceutical composition described herein, wherein the cancer is ovarian cancer, endometrial cancer, lung cancer, head and neck cancer, thyroid cancer, breast cancer, gastric cancer, kidney cancer, prostate cancer, liver cancer, pancreatic cancer, or colorectal cancer.

In a further aspect, provided is a method of treating cancer, wherein the cancer is ovarian cancer. In a further aspect, provided is a method of treating cancer, wherein the cancer is endometrial cancer. In a further aspect, provided is a method of treating cancer, wherein the cancer is lung cancer. In a further aspect, provided is a method of treating cancer, wherein the cancer is head and neck cancer. In a further aspect, provided is a method of treating cancer, wherein the cancer is thyroid cancer. In a further aspect, provided is a method of treating cancer, wherein the cancer is breast cancer. In a further aspect, provided is a method of treating cancer, wherein the cancer is gastric cancer. In a further aspect, provided is a method of treating cancer, wherein the cancer is kidney cancer. In a further aspect, provided is a method of treating cancer, wherein the cancer is prostate cancer. In a further aspect, provided is a method of treating cancer, wherein the cancer is liver cancer. In a further aspect, provided is a method of treating cancer, wherein the cancer is pancreatic cancer. In a further aspect, provided is a method of treating cancer, wherein the cancer is colorectal cancer.

In a further aspect, the patient being treated with an ADC or pharmaceutical composition described herein previously received a programmed death receptor-1 (PD-1) or programmed death-ligand 1 (PD-L1) inhibitor, with or without a platinum-containing chemotherapy in the neoadjuvant/adjuvant, locally advanced or metastatic setting.

In a further aspect, provided are methods comprising the administration of an effective amount of an ADC or pharmaceutical composition described herein in simultaneous, separate, or sequential combination with one or more antitumor agents. In a further aspect, provided are methods comprising the administration of an effective amount of an ADC or pharmaceutical composition described herein in simultaneous, separate, or sequential combination with a PD-1 inhibitor or PD-L1 inhibitor.

In another aspect, provided herein is a PTK-7 ADC or pharmaceutical composition described herein, for use in therapy. In a further aspect, provided is an ADC or pharmaceutical composition described herein, for use in the treatment of cancer. In a further aspect, the cancer is ovarian cancer, endometrial cancer, lung cancer, head and neck cancer, thyroid cancer, breast cancer, gastric cancer, kidney cancer, prostate cancer, liver cancer, pancreatic cancer, or colorectal cancer.

In a further aspect, provided herein is an ADC or pharmaceutical composition described herein, for use in the treatment of ovarian cancer. In a further aspect, provided herein is an ADC or pharmaceutical composition described herein, for use in the treatment of endometrial cancer. In a further aspect, provided herein is an ADC or pharmaceutical composition described herein, for use in the treatment of lung cancer. In a further aspect, provided herein is an ADC or pharmaceutical composition described herein, for use in the treatment of head and neck cancer. In a further aspect, provided herein is an ADC or pharmaceutical composition described herein, for use in the treatment of thyroid cancer. In a further aspect, provided herein is an ADC or pharmaceutical composition described herein, for use in the treatment of breast cancer. In a further aspect, provided herein is an ADC or pharmaceutical composition described herein, for use in the treatment of gastric cancer. In a further aspect, provided herein is an ADC or pharmaceutical composition described herein, for use in the treatment of kidney cancer. In a further aspect, provided herein is an ADC or pharmaceutical composition described herein, for use in the treatment of prostate cancer. In a further aspect, provided herein is an ADC or pharmaceutical composition described herein, for use in the treatment of liver cancer. In a further aspect, provided herein is an ADC or pharmaceutical composition described herein, for use in the treatment of pancreatic cancer. In a further aspect, provided herein is an ADC or pharmaceutical composition described herein, for use in the treatment of colorectal cancer.

In a further aspect, provided herein is an ADC or pharmaceutical composition described herein, for use in the treatment of cancer, wherein prior use occurred for a PD-1 or PD-L1 inhibitor, and with or without a platinum-containing chemotherapy in the neoadjuvant/adjuvant, locally advanced or metastatic setting.

In a further aspect, provided herein is an ADC or pharmaceutical composition described herein in simultaneous, separate, or sequential combination with one or more antitumor agents for use in the treatment of cancer. In a further aspect, with the antitumor agent is a PD-1 inhibitor or PD-L1 inhibitor.

In another aspect, provided herein is the use of a PTK-7 ADC or pharmaceutical composition described herein for the manufacture of a medicament for the treatment of cancer. In a further aspect, provided herein is the use of an ADC or pharmaceutical composition described herein for the manufacture of a medicament for the treatment of cancer, wherein the cancer is ovarian cancer, endometrial cancer, lung cancer, head and neck cancer, thyroid cancer, breast cancer, gastric cancer, kidney cancer, prostate cancer, liver cancer, pancreatic cancer, or colorectal cancer.

In a further aspect, provided herein is the use of an ADC or pharmaceutical composition described herein for the manufacture of a medicament for the treatment of cancer, wherein prior use of a PD-1 or PD-L1 inhibitor, and with or without a platinum-containing chemotherapy in the neoadjuvant/adjuvant, locally advanced or metastatic setting.

In a further aspect, provided herein is the use of an ADC or pharmaceutical composition described herein in the manufacture of a medicament for the treatment of cancer wherein said medicament is to be administered simultaneously, separately, or sequentially with one or more antitumor agents. In a further aspect, provided herein is the use of an ADC or pharmaceutical composition described herein in the manufacture of a medicament for the treatment of cancer wherein said medicament is to be administered simultaneously, separately, or sequentially with a PD-1 inhibitor or PD-L1 inhibitor.

In a further aspect, the breast cancer is HR-positive, HER2-negative breast cancer, or triple negative breast cancer (TNBC). In a further aspect, the breast cancer is ductal or lobular. In a further aspect, the lung cancer is squamous non-small cell lung cancer (NSCLC) or non-squamous NSCLC. In a further aspect, the NSCLC is metastatic or advanced. In a further aspect, the lung cancer is squamous, adenocarcinoma, or small cell carcinoma. In a further aspect, the lung cancer is small cell lung cancer (SCLC). In a further aspect, the small cell lung cancer is metastatic or advanced. In a further aspect, the gastric cancer is esophageal cancer or gastroesophageal junction cancer. In a further aspect, the ovarian cancer is metastatic or advanced ovarian carcinoma. In a further aspect, the ovarian cancer is serous or mucinous. In a further aspect, the ovarian cancer is fallopian or peritoneal. In a further aspect, the ovarian cancer is epithelial. In a further aspect, the patient with ovarian cancer has relapsed or refractory disease after treatment with one or a combination of paclitaxel, carboplatin, platinum-based chemotherapy, and mirvetuximab soravtansine.

In a further aspect, the patient or the cancer has relapsed or become refractory to one or more of the following standard of care treatments: carboplatin and etoposide with or without atezolizumab (or durvalumab), cisplatin and etoposide with or without atezolizumab (or durvalumab), pembrolizumab, pembrolizumab with or without carboplatin and pemetrexed, docetaxel with or without ramucirumab, carboplatin and paclitaxel with or without bevacizumab.

In a further aspect, the antitumor agents may be chemotherapeutic therapeutic agents, including platinum-containing chemotherapy, and/or may include cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), gemcitabine, topotecan, liposomal irinotecan, pemetrexed, and cetuximab. In a further aspect, the antitumor agents may be immuno-oncology agents, including those selected from the group consisting of nivolumab, ipilimumab, pidilizumab, pembrolizumab, tremelimumab, urelumab, lirilumab, atezolizumab, epacadostat, and durvalumab. In a further aspect, the antitumor agents may be carboplatin and etoposide with or without atezolizumab (or durvalumab), cisplatin and etoposide with or without atezolizumab (or durvalumab), pembrolizumab, pembrolizumab with or without carboplatin and pemetrexed, docetaxel with or without ramucirumab, carboplatin and paclitaxel with or without bevacizumab.

### Pharmaceutical Compositions and Methods of Administration

The antibodies or ADCs described herein can be formulated as pharmaceutical compositions administered by any route which makes the antibody or ADC bioavailable including, for example, oral, topical, or subcutaneous administration.

Also provided herein is a pharmaceutical composition comprising an antibody or ADC provided herein and one or more agents selected from the group consisting of a physiologically acceptable carrier, a diluent, an excipient, and an auxiliary.

The antibodies or ADCs of the present disclosure, or pharmaceutical compositions comprising the same, may be administered by parenteral routes (e.g., subcutaneous and intravenous). An antibody or ADC of the present disclosure may be administered to a patient alone with pharmaceutically acceptable carriers, diluents, or excipients in single or multiple doses. Pharmaceutical compositions described herein can be prepared by methods well known in the art (e.g., Remington: The Science and Practice of Pharmacy, 22nd ed. (2012), A. Loyd et al., Pharmaceutical Press) and comprise an antibody or ADC, as disclosed herein, and one or more pharmaceutically acceptable carriers, diluents, or excipients.

In an aspect, disclosed herein is a pharmaceutical composition comprising an antibody disclosed herein and one or more pharmaceutically acceptable carriers, diluents, or excipients. In an aspect, disclosed herein is a pharmaceutical composition comprising an ADC disclosed herein and one or more pharmaceutically acceptable carriers, diluents, or excipients.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

As used herein, the term “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

As used herein, the terms “treating”, “treatment”, or “to treat” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms.

The term “patient”, as used herein, refers to a human patient.

Certain abbreviations are defined as follows: “ACN” refers to acetonitrile; “AEEA” refers to 2-aminoethoxy-2-ethoxy acetic acid; “CTC” refers to 2-chlorotrityl chloride; “DCM” refers to dichloromethane; “DEA” refers to diethylamine; “DIC” refers to N,N′-diisopropylcarbodiimide; “DIPEA” refers to N,N-diisopropylethylamine; “DBU” refers to 1,8-diazabicyclo[5.4.0]undec-7-ene; “DMF” refers to N,N-dimethylformamide; “DTT” refers to dithiothreitol; “EtOAc” refers to ethyl acetate; “EDTA” refers to ethylenediaminetetraacetic acid, “EDCI” refers to 1-ethyl-3-(3-dimethylaminopropyl) carbodiimide; “FA” refers to formic acid; “h” refers to hour; “Fmoc” refers to fluorenylmethoxycarbonyl; “HEPES” refers to (N-2-hydroxyethylpiperazine-N′-2-ethanesulfonic acid); “HATU” refers to hexafluorophosphate azabenzotriazole tetramethyl uronium; “HBTU” refers to hexafluorophosphate benzotriazole tetramethyl uronium; “HOBt” refers to hydroxybenzotriazole; “HOPO” refers to 2-hydroxypyridine-1-oxide; “MeOH” refers to methanol; “MTBE” refers to methyl tert-butyl ether; “MWCO” refers to molecular weight cut-off; “NHS” refers to N-hydroxysuccinimide; “NMP” refers to (N-methyl-2-pyrrolidone); “OAll” refers to allyloxy; “Sar” refers to sarcosine; “Su” refers to succinimide; “THF” refers to tetrahydrofuran; “TsOH” refers to p-toluenesulfonic acid; and “TCEP” refers to (tris(2-carboxyethyl) phosphine).

### Embodiments

Embodiments of the present disclosure are contemplated to include, but are not limited to the following:

1. An antibody that binds human PTK-7, wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein:
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19;
c) the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29; or
d) the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

2. The antibody of embodiment 1, wherein the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9.

3. The antibody of embodiment 1, wherein the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19.

4. The antibody of embodiment 1, wherein the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29.

5. The antibody of embodiment 1, wherein the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

6. The antibody of embodiment 1, wherein:
a) the VH comprises SEQ ID NO: 10 and the VL comprises SEQ ID NO: 11;
b) the VH comprises SEQ ID NO: 20 and the VL comprises SEQ ID NO: 21;
c) the VH comprises SEQ ID NO: 30 and the VL comprises SEQ ID NO: 31;
d) the VH comprises SEQ ID NO: 39 and the VL comprises SEQ ID NO: 40.

7. The antibody of embodiment 6, wherein the antibody comprises a VH comprising SEQ ID NO: 10 and a VL comprising SEQ ID NO: 11.

8. The antibody of embodiment 6, wherein the antibody comprises a VH comprising SEQ ID NO: 20 and a VL comprising SEQ ID NO: 21.

9. The antibody of embodiment 6, wherein the antibody comprises a VH comprising SEQ ID NO: 30 and a VL comprising SEQ ID NO: 31.

10. The antibody of embodiment 6, wherein the antibody comprises a VH comprising SEQ ID NO: 39 and a VL comprising SEQ ID NO: 40.

11. The antibody of any one of embodiments 1-10, wherein the antibody has a human IgG1 or IgG4 isotype.

12. The antibody of embodiment 11, wherein the antibody has a human IgG1 isotype.

13. The antibody of embodiment 12, comprising alanine at residues 234 and 235 (according to EU Index numbering).

14. The antibody of embodiment 13, further comprising serine at position 265 (according to EU Index numbering).

15. The antibody of embodiment 1, wherein the antibody comprises a heavy chain (HC) and a light chain (LC), wherein:
a) the HC comprises amino acids 2-441 of SEQ ID NO: 2 and the LC comprises SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23; or
d) the HC comprises amino acids 2-444 of SEQ ID NO: 32 and the LC comprises amino acids 2-215 of SEQ ID NO: 33.

16. The antibody of embodiment 15, wherein:
a) the HC consists of SEQ ID NO: 2 and the LC consists of SEQ ID NO: 3;
b) the HC consists of SEQ ID NO: 12 and the LC consists of SEQ ID NO: 13;
c) the HC consists of SEQ ID NO: 22 and the LC consists of SEQ ID NO: 23; or
d) the HC consists of SEQ ID NO: 32 and the LC consists of SEQ ID NO: 33.

17. The antibody of embodiment 15 or 16, wherein the HC consists of SEQ ID NO: 2 and the LC consists of SEQ ID NO: 3.

18. The antibody of embodiment 15 or 16, wherein the HC consists of SEQ ID NO: 12 and the LC consists of SEQ ID NO: 13.

19. The antibody of embodiment 15 or 16, wherein the HC consists of SEQ ID NO: 22 and the LC consists of SEQ ID NO: 23.

20. The antibody of embodiment 15 or 16, wherein the HC consists of SEQ ID NO: 32 and the LC consists of SEQ ID NO: 33.

21. An antibody-drug conjugate (ADC) comprising the antibody of any one of embodiments 1-20 conjugated to a cytotoxic agent.

22. The ADC of embodiment 21, wherein the cytotoxic agent is selected from the group consisting of a microtubule inhibitor, a topoisomerase I inhibitor, a DNA damaging agent, a DNA alkylating agent, and a DNA minor groove binder.

23. The ADC of embodiment 21 or 22, wherein the cytotoxic agent is a topoisomerase I inhibitor.

24. The ADC of embodiment 23, wherein the topoisomerase I inhibitor is a camptothecin analog.

25. The ADC of embodiment 24, wherein the camptothecin analog comprises the Formula:

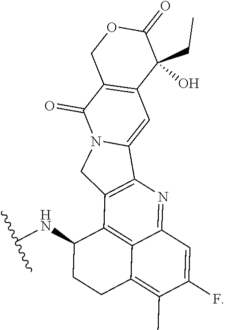 [SMILES: *N[C@@H]1CCc2c(C)c(F)cc3nc4c(c1c23)Cn1c-4cc2c(c1=O)COC(=O)[C@@]2(O)CC]

26. The ADC of embodiment 24 or 25, wherein the ADC comprises one of the Formula:

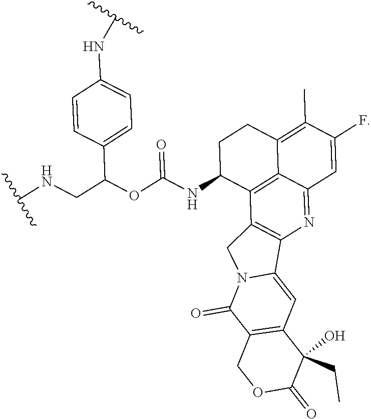 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(N*)cc1]

27. The ADC of any one of embodiments 21-26, wherein the ADC further comprises a linker which connects the antibody to the cytotoxic agent.

28. The ADC of embodiment 27, wherein the linker comprises a peptide unit or a sugar cleavable unit.

29. The ADC of embodiment 28, wherein the peptide unit comprises Val-Ala, Val-Cit, Phe-Lys, or Ala-Ala-Asn.

30. The ADC of embodiment 29, wherein the peptide unit comprises Val-Ala.

31. The ADC of embodiment 29, wherein the peptide unit comprises Val-Cit.

32. The ADC of any one of embodiments 21-30, wherein the ADC comprises the Formula:

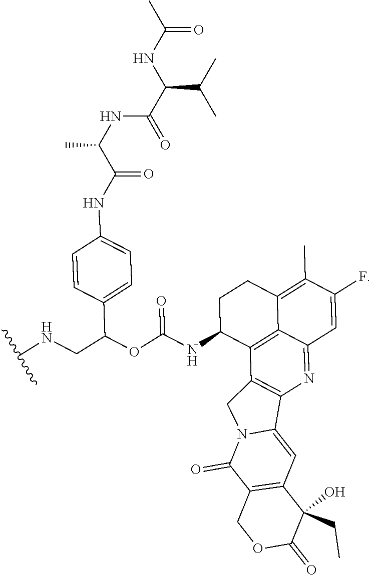 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(NC(=O)[C@H](C)NC(=O)[C@@H](NC(C)=O)C(C)C)cc1]

33. The ADC of embodiment 28, wherein the sugar cleavable unit comprises the Formula:

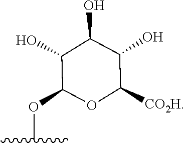 [SMILES: *O[C@@H]1O[C@H](C(=O)O)[C@@H](O)[C@H](O)[C@H]1O]

34. The ADC of embodiment 33, wherein the ADC comprises the Formula:

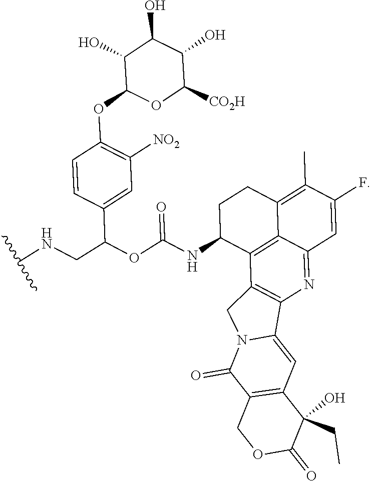 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(O[C@@H]2O[C@H](C(=O)O)[C@@H](O)[C@H](O)[C@H]2O)c([N+](=O)[O-])c1]

35. The ADC of any one of embodiments 21-34, wherein the linker further comprises a hydrophobicity masking group.

36. The ADC of embodiment 35, wherein the hydrophobicity masking group is selected from polysarcosine or polyethylene glycol.

37. The ADC of embodiment 35 or 36, wherein the hydrophobicity masking group is in a branched configuration on the linker.

38. The ADC of embodiment 36 or 37, wherein the hydrophobicity masking group is polysarcosine of the Formula:

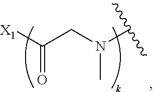

and wherein k is an integer from 6 to 12, and X₁ is H, OH, or NH₂.

39. The ADC of embodiment 38, wherein k is 10.

40. The ADC of embodiment 38, wherein k is 12.

41. The ADC of any one of embodiments 27-40, wherein the linker further comprises a connecting unit.

42. The ADC of embodiment 41, wherein the connecting unit is of the Formula:

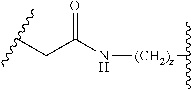

wherein z is from 1 to 5.

43. The ADC of embodiment 41, wherein the connecting unit is of the Formula:

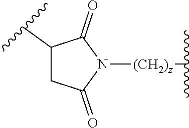

wherein z is from 1 to 5.

44. The ADC of embodiment 41, wherein the connecting unit is of the Formula:

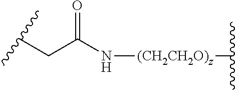

wherein z is from 1 to 5.

45. The ADC of embodiment 41, wherein the connecting unit is of the Formula:

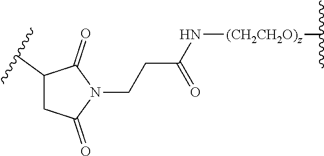

wherein z is from 1 to 5.

46. An antibody-drug conjugate (ADC), wherein the ADC is one of the Formula:

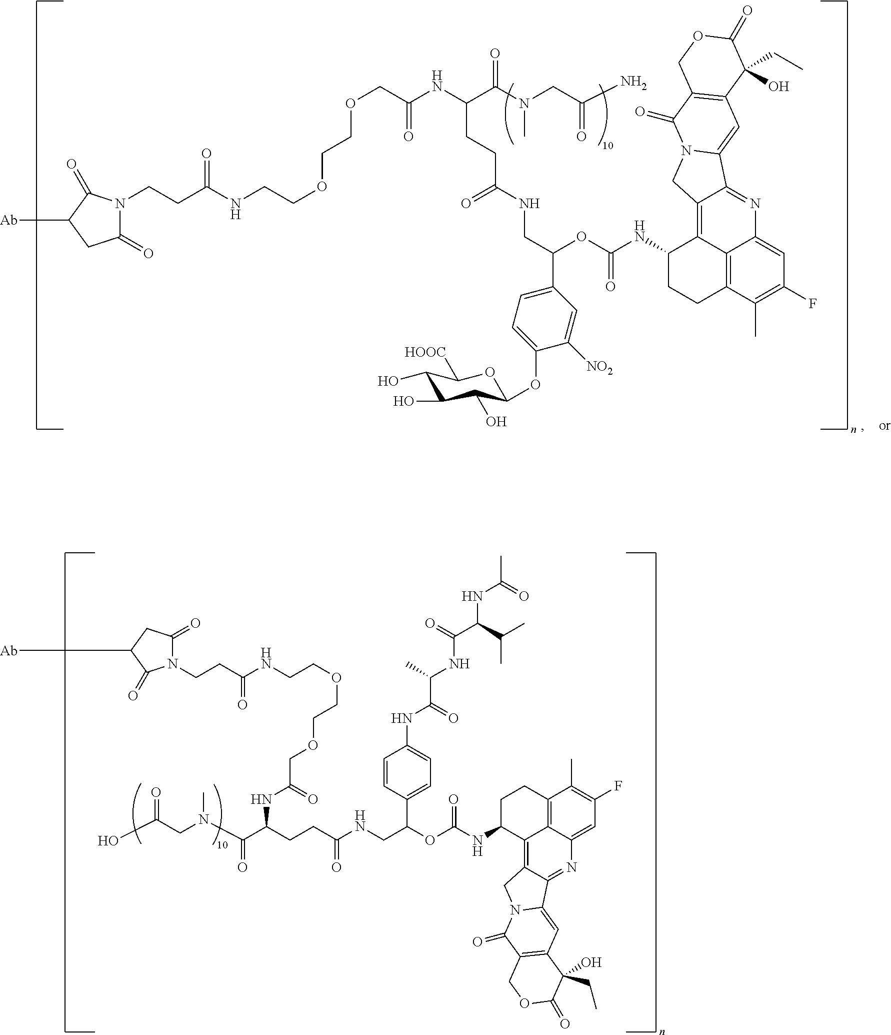

wherein:
Ab is the antibody of any one of embodiments 1-21, and
n is from about 1 to 16.

47. The ADC of embodiment 46, wherein n is from about 2 to 12.

48. The ADC of embodiment 46, wherein n is from about 2 to 8.

49. The ADC of any one of embodiments 46-48, wherein the ADC is of the Formula:

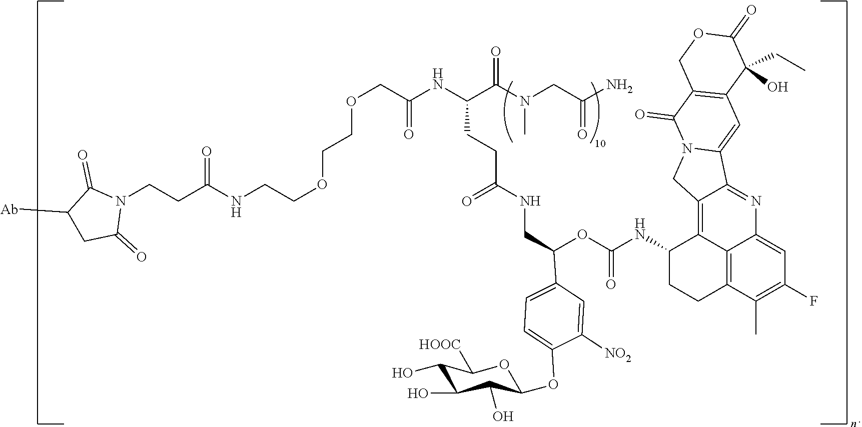

50. The ADC of any one of embodiments 46-48, wherein the ADC is of the Formula:

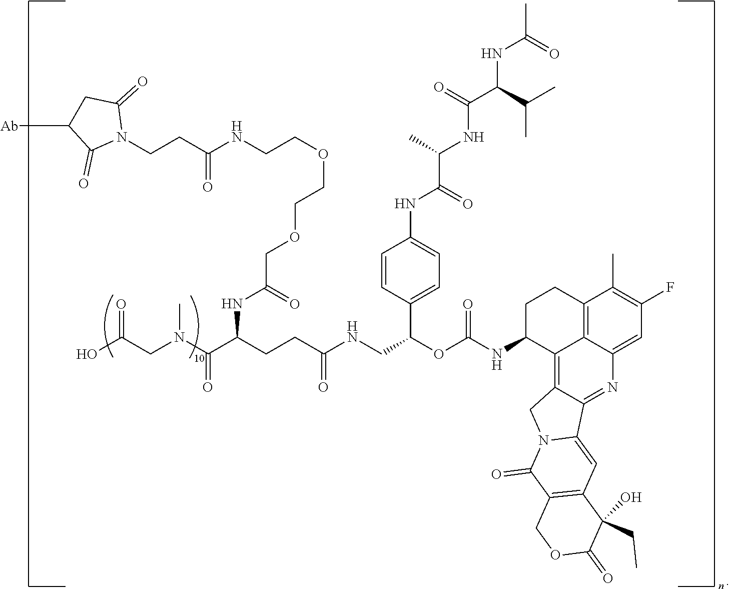

51. The ADC of any one of embodiments 46-50, wherein n is about 2.

52. The ADC of any one of embodiments 46-50, wherein n is about 4.

53. The ADC of any one of embodiments 46-50, wherein n is about 6.

54. The ADC of any one of embodiments 46-50, wherein n is about 8.

55. The ADC of any one of embodiments 46-54, wherein connection to the antibody occurs through a thiol group on one or more cysteines of the antibody.

56. The ADC of embodiment 55, wherein the one or more cysteines are each a natural cysteine in the hinge region of the antibody.

57. The ADC of any one of embodiments 46-56, wherein the Ab comprises a HC comprising amino acids 2-441 of SEQ ID NO: 2 and a LC comprising SEQ ID NO: 3, and wherein n is about 8.

58. The ADC of embodiment 57, wherein the Ab comprises a HC consisting of SEQ ID NO: 2 and a LC consisting of SEQ ID NO: 3.

59. The ADC of any one of embodiments 46-56, wherein the Ab comprises a HC comprising amino acids 2-448 of SEQ ID NO: 12 and a LC comprising amino acids 2-215 of SEQ ID NO: 13, and wherein n is about 8.

60. The ADC of embodiment 59, wherein the Ab comprises a HC consisting of SEQ ID NO: 12 and a LC consisting of SEQ ID NO: 13.

61. The ADC of any one of embodiments 46-56, wherein the Ab comprises a HC comprising amino acids 2-447 of SEQ ID NO: 22 and a LC comprising amino acids 2-215 of SEQ ID NO: 23, and wherein n is about 8.

62. The ADC of embodiment 61, wherein the Ab comprises a HC consisting of SEQ ID NO: 22 and a LC consisting of SEQ ID NO: 23.

63. The ADC of any one of embodiments 46-56, wherein the Ab comprising amino acids 2-444 of SEQ ID NO: 32 and a LC comprising amino acids 2-215 of SEQ ID NO: 33, and wherein n is about 8.

64. The ADC of embodiment 63, wherein the Ab comprises a HC consisting of SEQ ID NO: 32 and a LC consisting of SEQ ID NO: 33.

65. A pharmaceutical composition comprising the antibody of any one of embodiments 1-20 and one or more pharmaceutically acceptable carriers, diluents, or excipients.

66. A pharmaceutical composition comprising the ADC of any one of embodiments 21-64 and one or more pharmaceutically acceptable carriers, diluents, or excipients.

67. A method of treating cancer, comprising administering to a patient in need thereof, an effective amount of the ADC of any one of embodiments 21-64.

68. The method of embodiment 67, wherein the cancer is ovarian cancer, lung cancer, breast cancer, gastric cancer, kidney cancer, prostate cancer, liver cancer, or colorectal cancer.

69. The method of embodiment 67 or 68, further comprising administrating simultaneously, separately, or sequentially a PD-1 inhibitor or PD-L1 inhibitor.

70. The ADC of any one of embodiments 21-64, for use in therapy.

71. The ADC of any one of embodiments 21-64, for use in the treatment of cancer.

72. The ADC for use of embodiment 71, wherein the cancer is ovarian cancer, lung cancer, breast cancer, gastric cancer, kidney cancer, prostate cancer, liver cancer, or colorectal cancer.

73. The ADC for use of any one of embodiments 21-64, wherein the ADC is administered in simultaneous, separate, or sequential combination with a PD-1 inhibitor or PD-L1 inhibitor.

74. A pharmaceutical composition for use in treating cancer, comprising an effective amount of the ADC of any one of embodiments 21-64.

75. The composition for use of embodiment 74, wherein the cancer is ovarian cancer, lung cancer, breast cancer, gastric cancer, kidney cancer, prostate cancer, liver cancer, or colorectal cancer.

76. The composition of embodiment 74 or 75, which is administered in simultaneous, separate, or sequential combination with a PD-1 inhibitor or PD-L1 inhibitor.

77. The use of an ADC of any one of embodiments 21-64 for the manufacture of a medicament for the treatment of cancer.

78. The use of embodiment 77, wherein the cancer is ovarian cancer, lung cancer, breast cancer, gastric cancer, kidney cancer, prostate cancer, liver cancer, or colorectal cancer.

79. The use of embodiment 77 or 78, wherein said medicament further comprises a PD-1 inhibitor or PD-L1 inhibitor.

80. A method of preparing an ADC comprising conjugating the antibody of any one of embodiments 1-20 to a linker-payload.

81. The method of embodiment 80, wherein the linker-payload comprises the Formula:

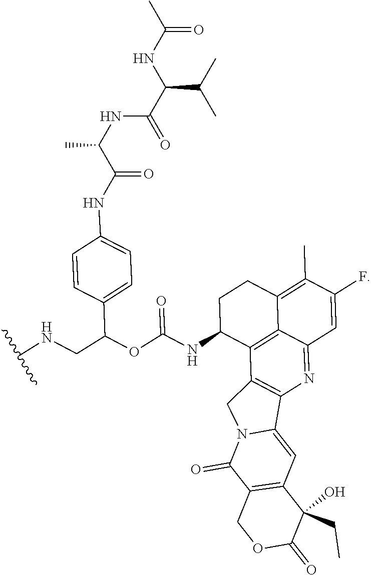 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(NC(=O)[C@H](C)NC(=O)[C@@H](NC(C)=O)C(C)C)cc1]

82. The method of embodiment 80, wherein the linker-payload comprises the Formula:

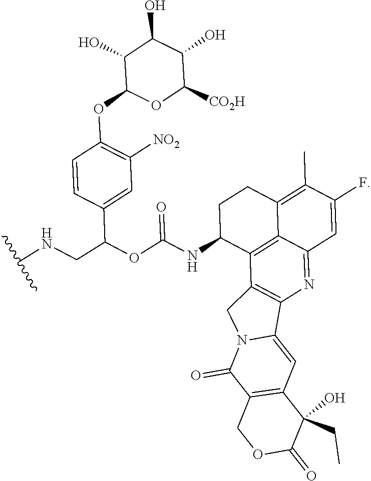 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(O[C@@H]2O[C@H](C(=O)O)[C@@H](O)[C@H](O)[C@H]2O)c([N+](=O)[O-])c1]

83. A method of producing an ADC, the method comprising contacting the antibody of any one of embodiments 1-20 with a compound of the Formula:

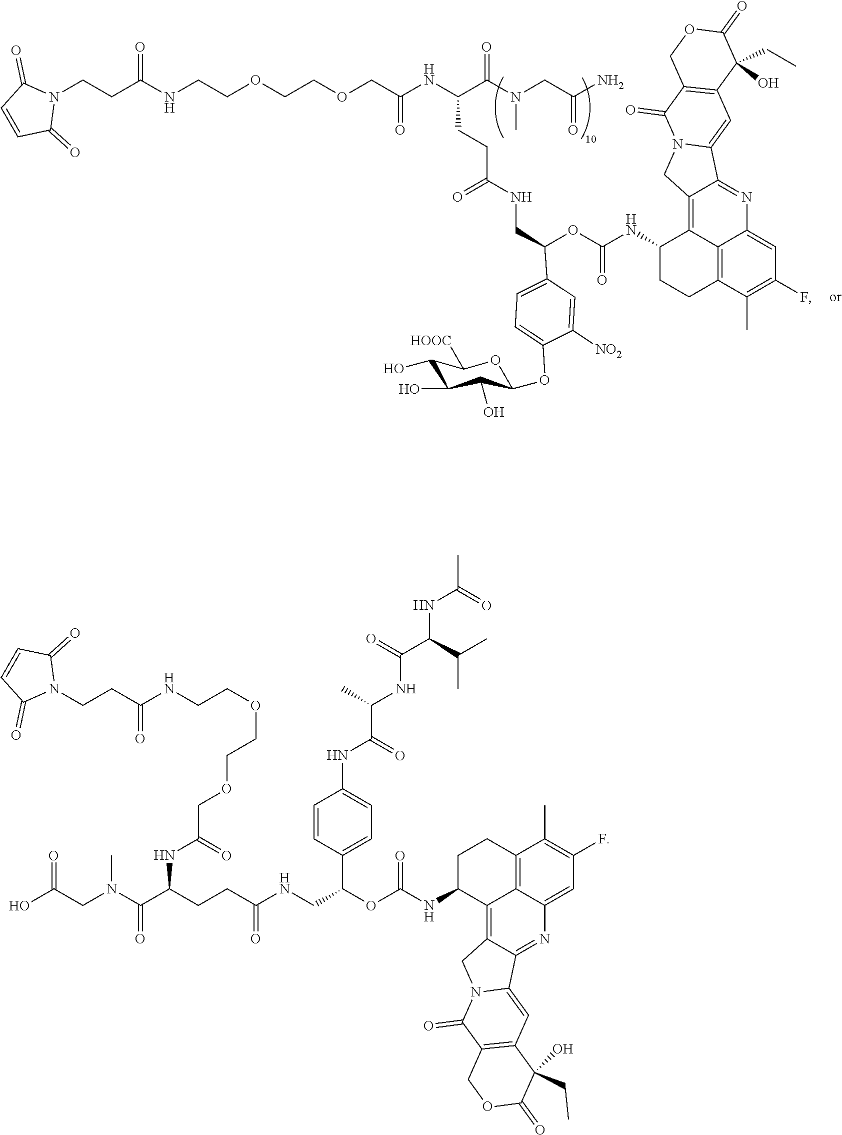

84. The method of embodiment 83, further comprising, reducing the antibody with a reducing agent to produce a reduced PTK-7 antibody prior to the contacting.

### EXAMPLES

### Example 1: Generation of PTK-7 Antibodies

The amino acid sequences of the CDRs, the variable regions, the complete heavy chain and light chain of Antibodies 1-4, and the nucleotide sequences encoding the same, are listed below in the section entitled “Amino Acid and Nucleotide Sequences.” In addition, the SEQ ID NOs for the CDRs, light chain, heavy chain, light chain variable region, and heavy chain variable region of Antibodies 1˜4 are shown in Tables 1 and 2.

The anti-PTK-7 antibodies of the present disclosure, including, but not limited to Abs 1-4, can be expressed and purified essentially as follows. Abs 1˜4 are fully human antibodies generated from immunization in a transgenic system.

Antibodies were synthesized and purified by well-known methods. Genes encoding for antibody heavy and light chain variable regions were cloned into PBv2-pKlight and pPB-huIgG1 (AAS) vectors for mAb expression and purification following standard procedures. Light chains are human kappa. Heavy chains are human IgG1-AAS: L234A/L235A/D265S based on EU index number.

An appropriate host cell, such as Chinese hamster ovarian cells (CHO), can be either transiently or stably transfected with an expression system for secreting antibodies using predetermined HC:LC ratios if two vectors are used, or either single vector system encoding both heavy and light chains. Clarified media, into which the antibody has been secreted, can be purified using the commonly used techniques. Initial capture utilizes Protein A column chromatography using conventional methods and buffer systems, followed by ion-exchange and/or hydrophobic interaction chromatography as polishing steps to remove host cell proteins and high molecule weight species. The product may be immediately frozen, for example at −80° C., or stored at 2-8° C. for several months.

TABLE 1
SEQ ID Nos for CDR amino acid sequences
of exemplified human PTK-7 antibodies

| PTK-7 | SEQ ID No. for CDR Sequence | | | | | |
|---|---|---|---|---|---|---|
| Antibody | HCDR1 | HCDR2 | HCDR3 | LCDR1 | LCDR2 | LCDR3 |
| Ab1 | 4 | 5 | 6 | 7 | 8 | 9 |
| Ab2 | 14 | 15 | 16 | 17 | 18 | 19 |
| Ab3 | 24 | 25 | 26 | 27 | 28 | 29 |
| Ab4 | 34 | 35 | 36 | 37 | 18 | 38 |

TABLE 2
SEQ ID Nos for exemplified human PTK-7 antibodies

| PTK-7 | | | | |
|---|---|---|---|---|
| Antibody | HC | LC | VH | VL |
| Ab1 | 2 | 3 | 10 | 11 |
| Ab2 | 12 | 13 | 20 | 21 |
| Ab3 | 22 | 23 | 30 | 31 |
| Ab4 | 32 | 33 | 39 | 40 |

TABLE 3
SEQ ID Nos for DNA sequences for heavy and light
chains of exemplified human PTK-7 antibodies

| PTK-7 | | |
|---|---|---|
| Antibody | HC | LC |
| Ab1 | 41 | 42 |
| Ab2 | 43 | 44 |
| Ab3 | 45 | 46 |
| Ab4 | 47 | 48 |

### Example 2: Generation of PTK-7 ADCs

### Synthesis of PSAR-Glucuronide-Exatecan-Containing Linker-Payload of Formula XIV

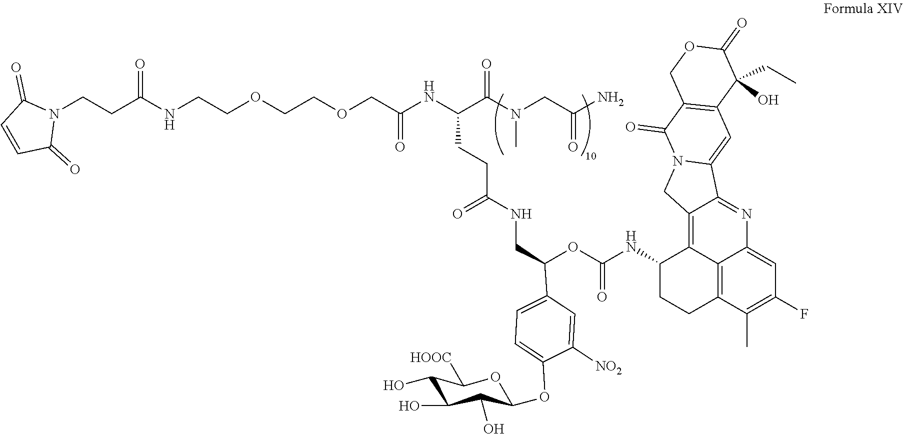

To prepare for conjugation to PTK antibodies of the present disclosure, the linker-payload of Formula XVI was synthesized from precursors or intermediates that include: a polysarcosine (PSAR) compound; a 4-beta-glucuronide-3-nitro-octapamine compound; an exatecan compound; and a maleimido-proprionyl compound. The linker-payload may be synthesized using methods disclosed in WO2019081455 and WO2022207699.

### PSAR Intermediate: FmocNH-PEG2-Glu (Su)-PSAR10-NH₂

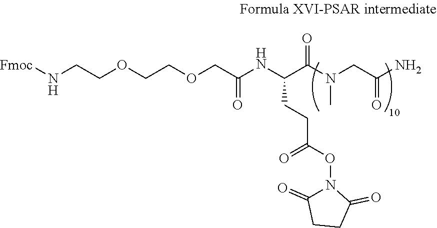

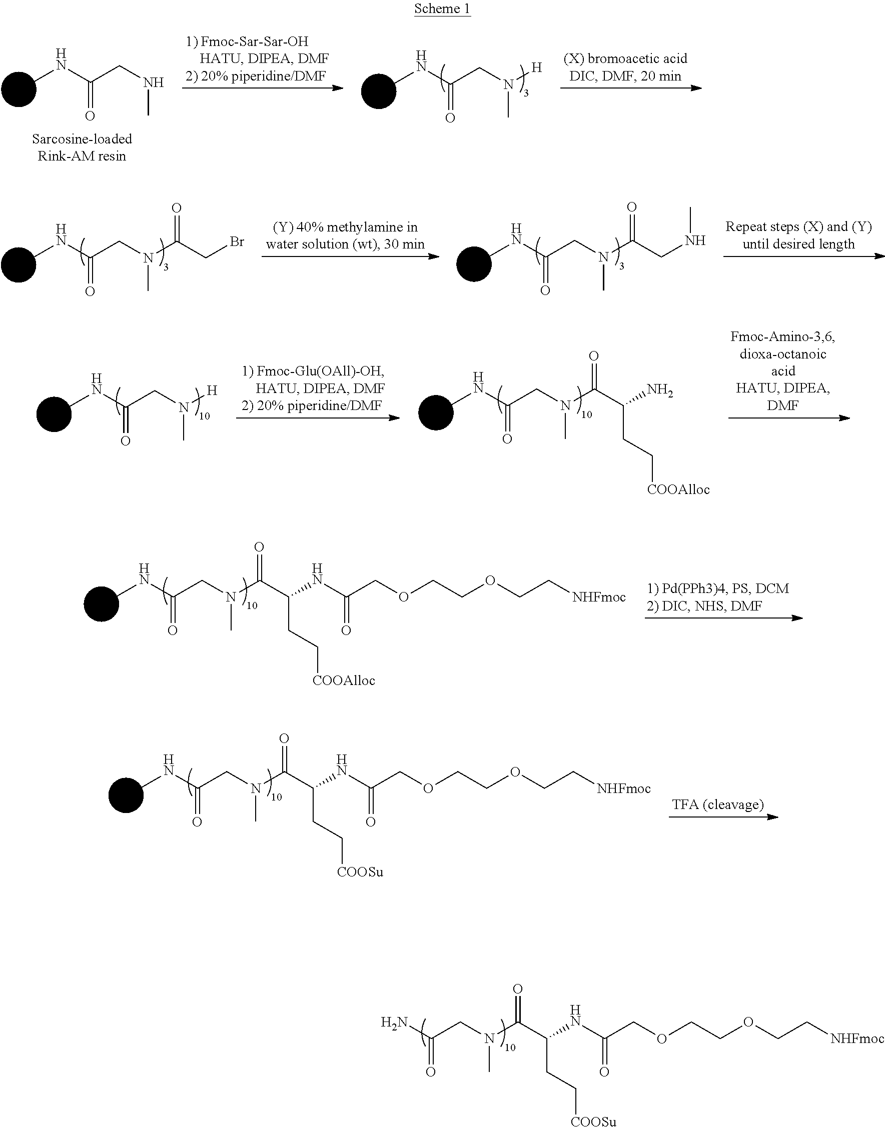

The PSAR intermediate may be prepared using Scheme 1. On-resin synthesis of polysarcosines is performed using sub-monomer synthesis iterative procedures for Rink amide with the commercial Fmoc-Sar-Sar-OH dipeptoid building block. Unless otherwise indicated, all reactions are performed at room temperature.

Rink amide preloaded with a first Fmoc-sarcosine residue is used as starting material. Fmoc-sarcosine preloaded Rink amide resin is treated with 20% piperidine in DMF (1 mL per 100 mg of resin) for 2×, 15 min at room temperature. The resin then is washed with DMF (4×) and DCM (4×). To the resin is added a solution of Fmoc-Sar-Sar-OH (3 eq), HATU (2.9 eq) and DIPEA (6 eq) in DMF (1 mL per 100 mg of resin). The reaction vessel is agitated for 2 hours and the resin is washed with DMF (4×) and DCM (4×). The resin is treated with 20% piperidine in DMF (1 mL per 100 mg of resin) for 2 times 15 min, at room temperature. The resin then is washed with DMF (4×) and DCM (4×) to provide a Rink resin having an n=3 polysarcosine oligomer.

Elongation of the n=3 polysarcosine oligomer is performed using sub-monomer synthesis procedures until the desired length is obtained via alternating bromoacetylation and amine displacement steps. The bromoacetylation step is performed by adding 10 eq of bromoacetic acid and 13 eq of diisopropylcarbodiimide in DMF (2 mL per 100 mg of resin). The mixture is agitated for 30 min, drained, and washed with DMF (4×). For the amine displacement step, a 40% (wt) methylamine in water solution is added (1.5 mL per 100 mg of resin) and the vessel is shaken for 30 min, drained, and washed with DMF (4×) and DCM (4×).

When the desired polysarcosine oligomer length is reached (e.g., a PSAR 10-mer), orthogonal chemical functionalization is performed. Orthogonal chemical functionalization may be followed by a final capping with a Fmoc-protected amino acid group or other group. The Fmoc protecting group may be removed before or after resin cleavage.

The polysarcosine may be functionalized with glutamic acid and Amino-3,6 dioxaoctanoic acid as follows. Fmoc-Glu (OAll)-OH (3 eq), HATU (2.9 eq) and DIPEA (6 eq) in DMF (1 mL per 100 mg of resin) are added to the Rink resin. The reaction vessel is agitated for 90 min and the resin is washed with DMF (4×) and DCM (4×). Resin then is treated with 20% piperidine in DMF (1 mL per 100 mg of resin) for 2×, 15 min at room temperature. The resin is washed with DMF (4×) and DCM (4×) followed by a 1-hour coupling with Fmoc-Amino-3,6 dioxaoctanoic acid (3 eq), HATU (2.9 eq), DIPEA (6 eq) in DMF (1 mL per 100 mg of resin). The resin is washed with DMF (4×), DCM (4×). The alloc-protecting group is removed by a 2×, 30 min treatment with a DCM solution containing 0.25 eq of Pd(PPh₃)₄ and 20 eq of phenylsilane (gently agitated under a stream of argon). The resin then is washed with DMF (5×) and DCM (5×). An N-hydroxysuccinimide (NHS) ester is introduced to the carboxylic acid side chain of the final polysarcosine compound by a 90 min treatment with a DMF solution containing 50 eq of DIC and 60 eq of N-hydroxysuccinimide (1.5 mL per 100 mg of resin). The resin then is washed with DMF (4×) and DCM (4×). Final polysarcosine compounds then are cleaved from the resin (100% TFA 2×, 30 min).

### 4-beta-glucuronide-3-nitro-octopamine intermediate: (2S,3R,4S,5S,6S)-2-(4-(2-((tert-butoxycarbonyl)amino)-1-(((4-nitrophenoxy) carbonyl)oxy)ethyl)-2-nitrophenoxy)-6-(methoxycarbonyl)tetrahydro-2H-pyran-3,4,5-triyl triacetate

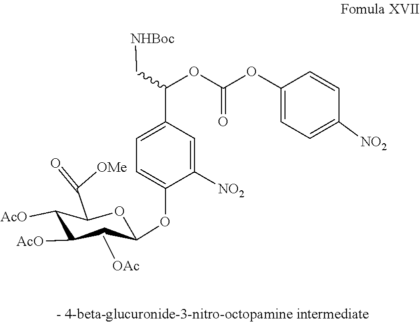

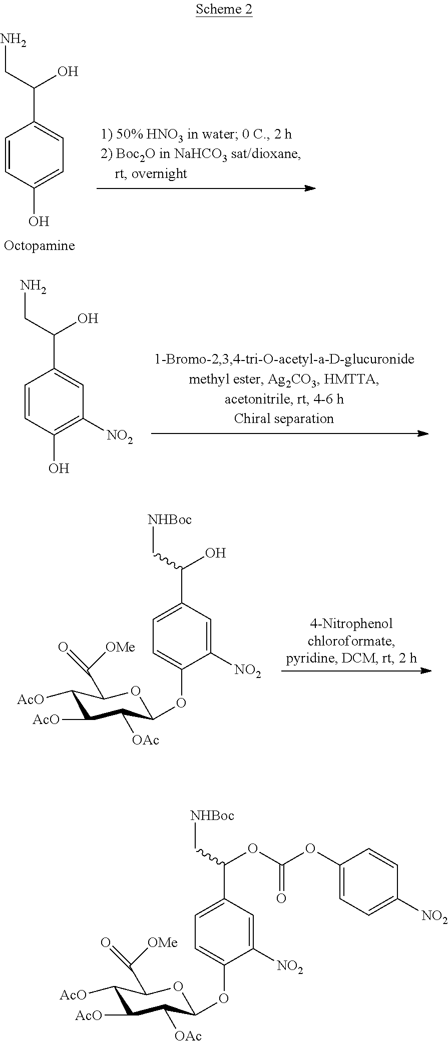

The 4-beta-glucuronide-3-nitro-octopamine intermediate 4-beta-glucuronide-3-nitro-octopamine intermediate: (2S,3R,4S,5S,6S)-2-(4-(2-((tert-butoxycarbonyl)amino)-1-(((4-nitrophenoxy) carbonyl)oxy)ethyl)-2-nitrophenoxy)-6-(methoxycarbonyl)tetrahydro-2H-pyran-3,4,5-triyl triacetate may be prepared using Scheme 2.

Octopamine or octopamine intermediates as utilized in the present methods may be obtained as racemic mixtures or as enantiopure compounds. Racemic mixtures may be subjected to chiral separation as known in the art to obtain enantiopure compounds.

Octopamine (±) hydrochloride (1690 mg, 11 mmol) is suspended in 4 mL of distilled water. The flask is chilled at 0° C. and 4 mL of a pre-chilled 65% nitric acid solution is slowly added. The reaction is kept at 0° C. for 20 minutes and mono-nitration is assessed by HPLC. The mono-nitrated octopamine precursor is transferred to a 250 mL pre-chilled Erlenmeyer flask and slowly neutralized at 0° C. with a saturated NaHCO₃ solution until a pH value of 8-9 is reached. 30 mL of dioxane then is added, followed by Boc₂O (7202 mg/13.2 mmol). The reaction is allowed to reach room temperature and is stirred overnight. The reaction then is diluted with EtOAc and washed 3× with a saturated citric acid solution and once with a saturated NaCl solution. The organic phase is dried over MgSO₄, filtered, and evaporated under vacuum to afford a crude product that is purified by chromatography on silica gel (petroleum ether/EtOAc, gradient from 70:30 to 20:80) to provide the mono-nitrated octopamine precursor: tert-butyl (2-hydroxy-2-(4-hydroxy-3-nitrophenyl)ethyl) carbamate. The resulting mono-nitrated octopamine precursor then is subjected to chiral separation prior to performing further synthesis steps.

Chiral separation of racemic tert-butyl (2-hydroxy-2-(4-hydroxy-3-nitrophenyl)ethyl) carbamate is performed using an MPLC (medium pressure liquid chromatography) column and a mobile phase of DCM±0.2% (v/v) EtOH (isocratic gradient) and sample solvent of DCM±0.2% (v/v) EtOH. To determine absolute configuration, phenolic position of both enantiomers is esterified with 1.2 molar equivalents of 4-nitrobenzoyl chloride and 2 molar equivalents of triethylamine in anhydrous THF. Compounds are purified by chromatography on silica gel (petroleum ether/EtOAc, gradient from 90:10 to 10:90) to provided 4-(2-((tert-butoxycarbonyl)amino)-1-hydroxyethyl)-2-nitrophenol 4-nitrobenzoate. Absolute configuration of enantiomers (previously dissolved in a 1:1 mixture of heptane/dichloromethane and allowed to slowly evaporate for 3 weeks to induce the formation of crystals) is confirmed by x-ray crystallography.

In a round-bottom flash, Ag₂CO₃ (1500 mg, 5.4 mmol) and 1,1,4,7,10,10-hexamethyltriethylenetetramine (251 mg, 1.1 mmol) are suspended in 4 mL of anhydrous acetonitrile and stirred for 2 hours at room temperature. Enantiopure tert-butyl (2-hydroxy-2-(4-hydroxy-3-nitrophenyl)ethyl) carbamate (292 mg, 0.98 mmol) and 1-bromo-2,3,4-tri-O-acetyl-a-D-glucuronide methyl ester (583 mg, 1.46 mmol) are added at 0° C. and the solution mixture is stirred for 4 h at room temperature. The reaction then is filtered on diatomaceous earth, diluted with EtOAc, and washed 3 times with a saturated citric acid solution and once with a saturated NaCl solution. The organic phase is dried over MgSO₄, filtered and evaporated under vacuum to afford a crude product that is purified by chromatography on silica gel (petroleum ether/EtOAc, gradient from 70:30 to 30:70) to provide (2S,3R,4S,5S,6S)-2-(4-(2-((tert-butoxycarbonyl)amino)-1-hydroxyethyl)-2-nitrophenoxy)-6-(methoxycarbonyl)tetrahydro-2H-pyran-3,4,5-triyl triacetate.

(2S,3R,4S,5S,6S)-2-(4-(2-((tert-butoxycarbonyl)amino)-1-hydroxyethyl)-2-nitrophenoxy)-6-(methoxycarbonyl)tetrahydro-2H-pyran-3,4,5-triyl triacetate (334 mg/0.54 mmol) and 4-nitrophenyl chloroformate (219 mg/1.09 mmol) are dissolved in 6 mL of dry DCM at 0° C. Anhydrous pyridine (112 mg, 1.41 mmol) is added and the mixture is stirred 30 min at room temperature. The reaction is filtered over a 0.45-um PTFE filter and purified by chromatography on silica gel (petroleum ether/EtOAc, gradient from 85:15 to 30:70) to provide (2S,3R,4S,5S,6S)-2-(4-(2-((tert-butoxycarbonyl)amino)-1-(((4-nitrophenoxy) carbonyl)oxy)ethyl)-2-nitrophenoxy)-6-(methoxycarbonyl)tetrahydro-2H-pyran-3,4,5-triyl triacetate.

### 4-beta-glucuronide-3-nitro-octopamine-exatecan intermediate: (2S,3R,4S,5S,6S)-2-(4-(2-amino-1-((((1R,9R)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl) carbamoyl)oxy)ethyl)-2-nitrophenoxy)-3,4,5-trihydroxytetrahydro-2H-pyran-2-carboxylic acid

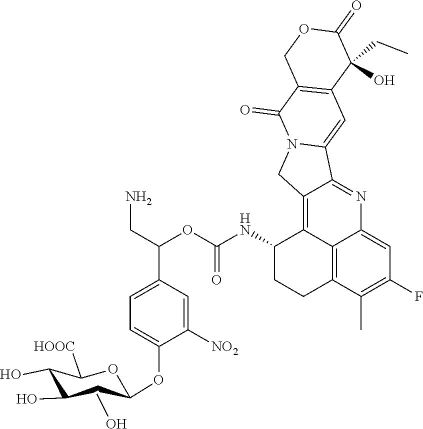

Formula XVIII-4-beta-glucuronide-3-nitro-octopamine-exatecan Intermediate

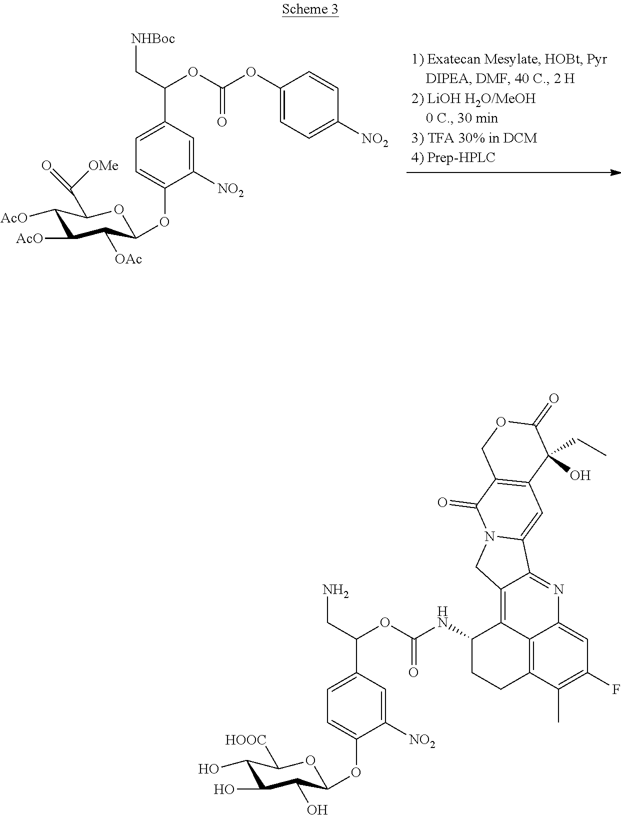

The 4-beta-glucuronide-3-nitro-octopamine intermediate may be conjugated to exatecan using Scheme 3 to provide the 4-beta-glucuronide-3-nitro-octopamine-exatecan intermediate: (2S,3R,4S,5S,6S)-2-(4-(2-amino-1-((((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy)ethyl)-2-nitrophenoxy)-3,4,5-trihydroxytetrahydro-2H-pyran-2-carboxylic acid.

101 mg (0.13 mmol) of 4-beta-glucuronide-3-nitro-octopamine intermediate (2S,3R,4S,5S,6S)-2-(4-(2-((tert-butoxycarbonyl)amino)-1-(((4-nitrophenoxy) carbonyl)oxy)ethyl)-2-nitrophenoxy)-6-(methoxycarbonyl)tetrahydro-2H-pyran-3,4,5-triyl triacetate, 0.14 mmol of Exatecan Mesylate, and 18 mg (0.13 mmol) of HOBt are dissolved in 1 mL of a 85:15 (v/v) mixture of anhydrous DMF/pyridine. 16.7 mg (0.13 mmol) of DIPEA is added. The reaction is stirred for 2 hours at 40° C. and volatiles are evaporated under reduced pressure. The crude residue is purified by chromatography on silica gel (DCM/MeOH gradient from 99:1 to 95:5) to provide an intermediate compound that next is subjected to deprotection.

144 mg (0.106 mmol) of this intermediate compound is dissolved in 3 mL of MeOH (75:25) at 0° C. LiOH monohydrate (44.5 mg, 1.06 mmol) is dissolved in water (0.4 mL) and is added to the reaction vessel. After stirring, the mixture is neutralized with acetic acid (83 mg, 1.4 mmol) and concentrated under reduced pressure. The obtained crude product is re-dissolved with a TFA/DCM solution and stirred. Volatiles are evaporated under reduced pressure, the crude residue is taken up and purified using HPLC to provide (2S,3R,4S,5S,6S)-2-(4-(2-amino-1-((((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy)ethyl)-2-nitrophenoxy)-3,4,5-trihydroxytetrahydro-2H-pyran-2-carboxylic acid.

### Conjugation of PSAR Intermediate and 4-Beta-Glucuronide-3-Nitro-Octopamine-Exatecan Intermediate to Provide Functionalized PSAR-4-Beta-Glucuronide-3-Nitro-Octopamine-Exatecan Intermediate

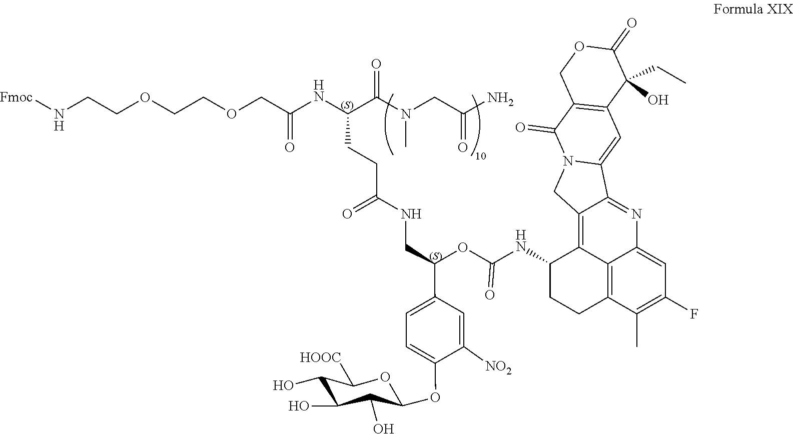

### Formula XIX-Functionalized PSAR-4-Beta-Glucuronide-3-Nitro-Octopamine-Exatecan Intermediate

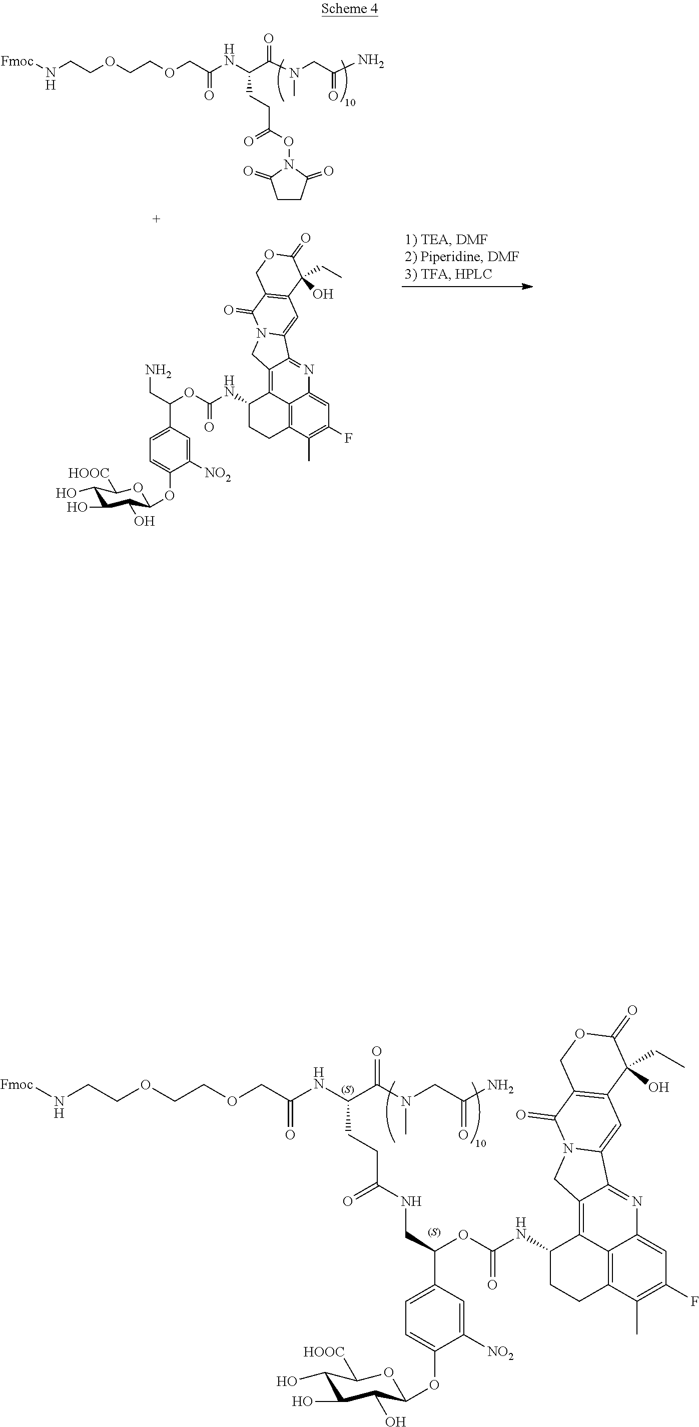

The PSAR intermediate and the 4-beta-glucuronide-3-nitro-octopamine-exatecan intermediate may be conjugated using Scheme 4 to provide the functionalized PSAR-4-beta-glucuronide-3-nitro-octopamine-exatecan intermediate.

100 mg (0.081 mmol) of the PSAR intermediate and 51 mg (0.061 mmol) of the 4-beta-glucuronide-3-nitro-octopamine-exatecan intermediate are dissolved in anhydrous DMF. 41 mg (0.405 mmol) of triethylamine is added and the reaction is stirred 30 min at room temperature. After entire conversion of the reaction is assessed by HPLC, piperidine is directly added into the reaction vial in order to reach an 8% (v/v) piperidine solution in DMF. The reaction is then stirred at room temperature 5-10 min, until entire Fmoc-deprotection is observed by HPLC. The reaction is slowly neutralized with a 10% TFA solution in water/ACN 1:1 (v/v) and purified using HPLC to provide the functionalized-PSAR-4-beta-glucuronide-3-nitro-octopamine-exatecan intermediate.

### Linker-Payload of Formula XIV: (2S,3S,4S,5R,6S)-6-(4-((3S,9S)-40-amino-9-(2-(2-(2-(3-(2,5-dioxo-2,5-dihydro-1H-pyrrol-1-yl) propanamido) ethoxy) ethoxy) acetamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-11,14,17,20,23,26,29,32,35,38-decamethyl-1,6,10,13,16,19,22,25,28,31,34,37,40-tridecaoxo-2-oxa-5,11,14,17,20,23,26,29,32,35,38-undecaazatetracontan-3-yl)-2-nitrophenoxy)-3,4,5-trihydroxytetrahydro-2H-pyran-2-carboxylic acid

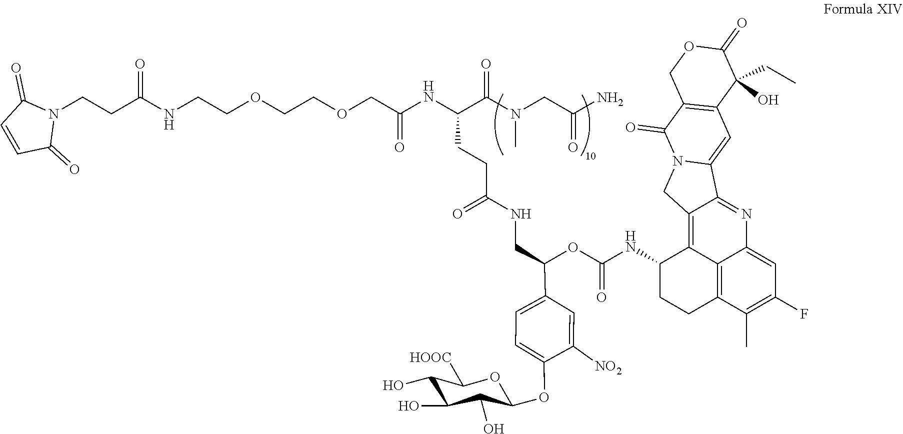

### Linker-Payload of Formula XIV

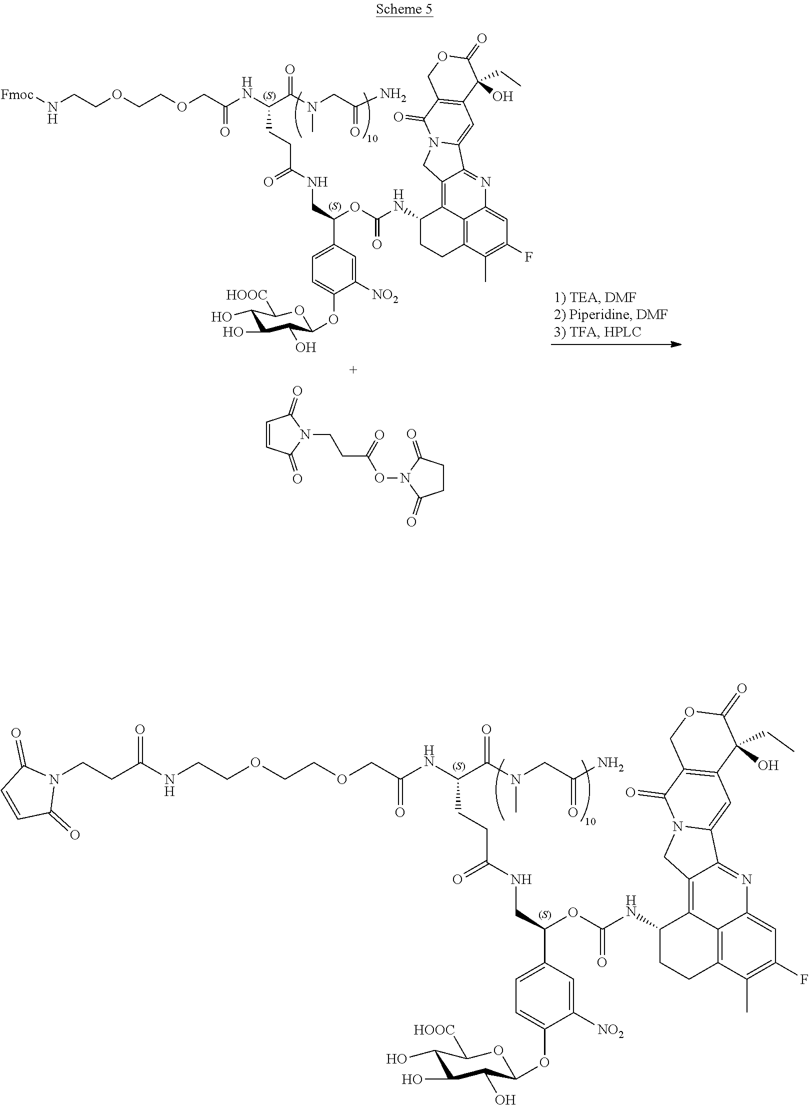

The Linker-Payload of Formula XIV may be prepared by conjugating maleimidopropionic acid N-hydroxysuccinimide ester and the functionalized-PSAR-4-beta-glucuronide-3-nitro-octopamine-exatecan intermediate using Scheme 5.

Maleimidopropionic acid N-hydroxysuccinimide ester and functionalized-PSAR-4-beta-glucuronide-3-nitro-octopamine-exatecan intermediate are dissolved in anhydrous DMF (0.1 M concentration of maleimide compound). 1.56 mg (0.015) of triethylamine was added and the reaction was stirred for 2 hours until entire conversion of the reaction as observed by HPLC. The reaction mixture is then diluted with a 1% TFA solution in water/ACN 1:1 (v/v) and purified using HPLC preparative method 6 to provide Linker-Payload of Formula XIV.

### Synthesis of PSAR-Val-Ala-Exatecan-Containing Linker-Payload of Formula XV

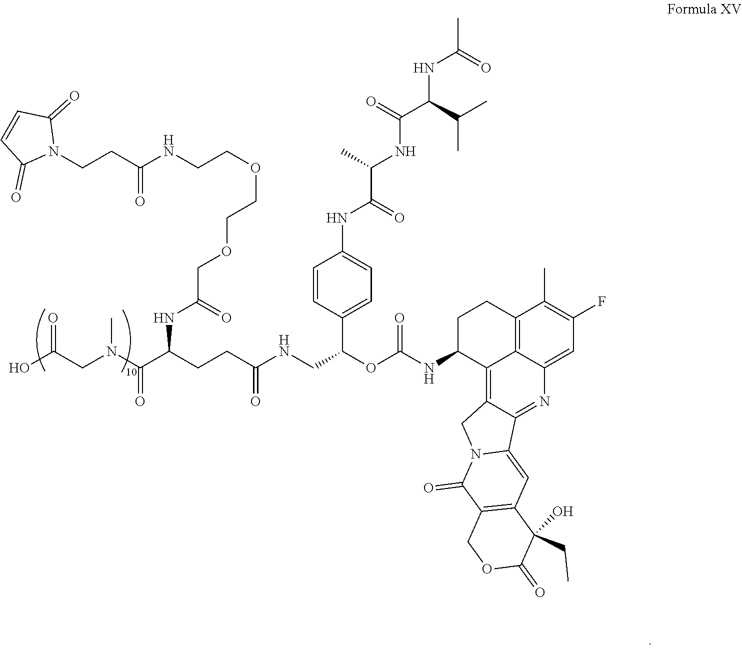

To prepare for conjugation to PTK antibodies of the present disclosure, the linker-payload of Formula XV was synthesized from precursors or intermediates that include: a polysarcosine (PSAR) compound; a valine-alanine compound; an exatecan compound; and a maleimido-compound. The linker-payload may be synthesized using methods disclosed in WO2019081455 and WO2022207699.

### (S)-32-(3-(((S)-2-(4-((S)-2-((S)-2-Acetamido-3-methylbutanamido) propanamido)phenyl)-2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy)ethyl)amino)-3-oxopropyl)-45-(2,5-dioxo-2,5-dihydro-1H-pyrrol-1-yl)-3,6,9,12,15,18,21,24,27,30-decamethyl-4,7,10,13,16,19,22,25,28,31,34,43-dodecaoxo-36,39-dioxa-3,6,9,12,15,18,21,24,27,30,33,42-dodecaazapentatetracontanoic acid

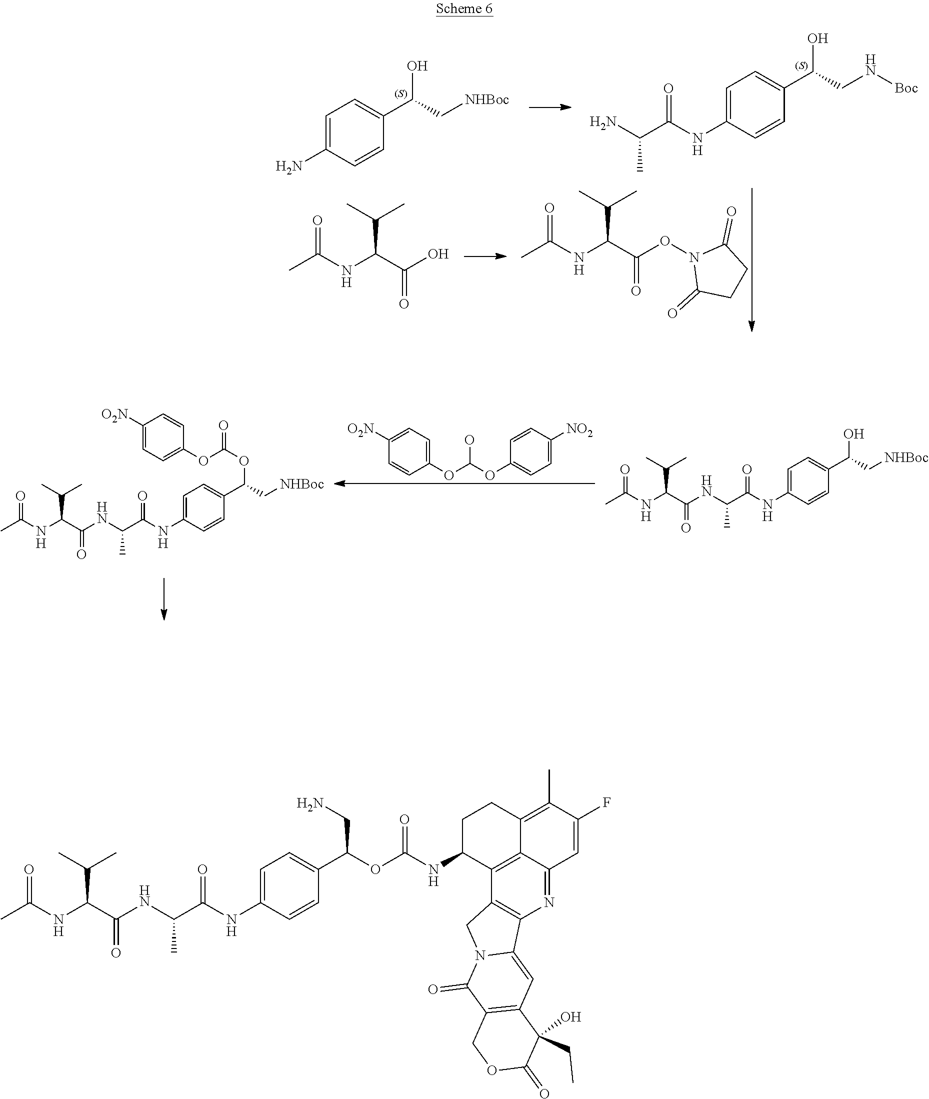

### Step 6.1a

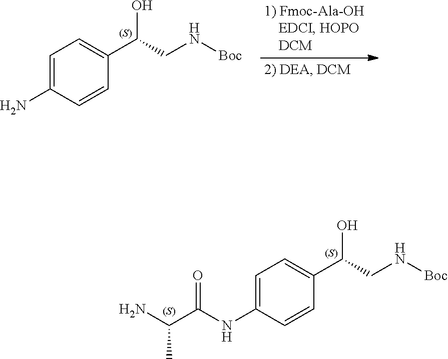

### tert-Butyl((S)-2-(4-((S)-2-aminopropanamido)phenyl)-2-hydroxyethyl)carbamate

Fmoc-Ala-OH (473.5 g. 1.52 mmol, 1.3 eq) was solubilized in DCM (3000 mL). The solution was cooled to 10° C. HOPO (154.2 g, 1.39 mmol, 1.6 eq) and EDCI (291.3 g, 1.88 mmol, 1.2 eq) were added under N₂. After stirring at 10° C. for 0.5 h, tert-butyl(S)-(2-(4-aminophenyl)-2-hydroxyethyl) carbamate (295.0 g, 1.17 mmol, 1 eq) was added and stirred 1 h at 15° C. DEA (1.67 kg, 19.5 eq) was added dropwise over 1 h. After completion of addition, the mixture was stirred 1 h at 15° C. and then 14 h at 0° C. The mixture was concentrated under vacuum (35° C.). Chromatography: eluent: n-heptane/DCM=10/1-1/10; DCM/THF=10/1-1/10; eluent: THF/MeOH=100/1-10/1. After evaporation under reduced pressure, title compound (270.0 g, 71%) was obtained.

### Step 6.1b

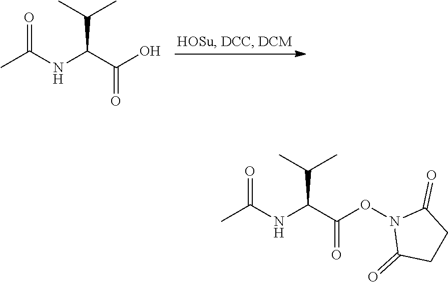 [SMILES: CC(=O)N[C@H](C(=O)O)C(C)C ; CC(=O)N[C@H](C(=O)ON1C(=O)CCC1=O)C(C)C]

### 2,5-Dioxopyrrolidin-1-yl acetyl-L-valinate

Ac-Val-OH (60.0 g, 1 eq) was solubilized in DCM (1200 mL), then addition of NHS (52.1 g, 1.2 eq) and stirred at 0° C. for 18 h. Added in one portion of DCC (93.3 g, 1.2 eq) and stirred at 0° C. for 18 h. Filtered, washing 3 times with DCM, and evaporated (40° C.) to half volume. Cooled to 30° C. and stirred for 1 h. Filtered, washing cake with 1:2 DCM/MTBE (50 mL), and drying under vacuum (30° C.) for 22 h. Obtained title compound (86.7 g. 89.8%) as a solid.

### Step 6.2

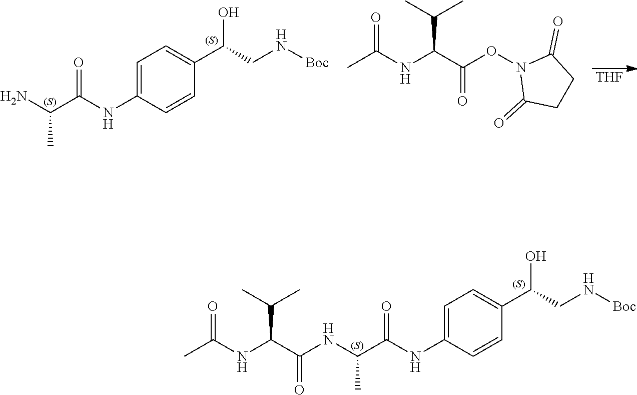

### tert-Butyl ((S)-2-(4-((S)-2-((S)-2-acetamido-3-methylbutanamido) propanamido)phenyl)-2-hydroxyethyl)carbamate

tert-Butyl ((S)-2-(4-((S)-2-aminopropanamido)phenyl)-2-hydroxyethyl) carbamate (17.0 g, 52.6 mmol, 1 eq) was solubilized in THF (250 mL). Temperature was adjusted to 10 to 20° C. 2,5-Dioxopyrrolidin-1-yl acetyl-L-valinate (13.47 g, 52.6 mmol, 1.0 eq) was added. Stirred at 10 to 20° C. for 3 h. Temperature was adjusted to −5 to 5° C. The reaction mixture was filtered, and the filter cake was dried for 14 h under N₂ flow at 10 to 20° C. The filter cake was taken up in THF (100 mL) and H₂O (350 mL) was added. Temperature was adjusted to 10 to 20° C. and stirred for 13 h. Filtered and washed filter cake with H₂O) (60 mL). Dried filter cake at 40 to 50° C. for 54 h. Obtained title compound (21 g, 86%) as a solid.

### Step 6.3

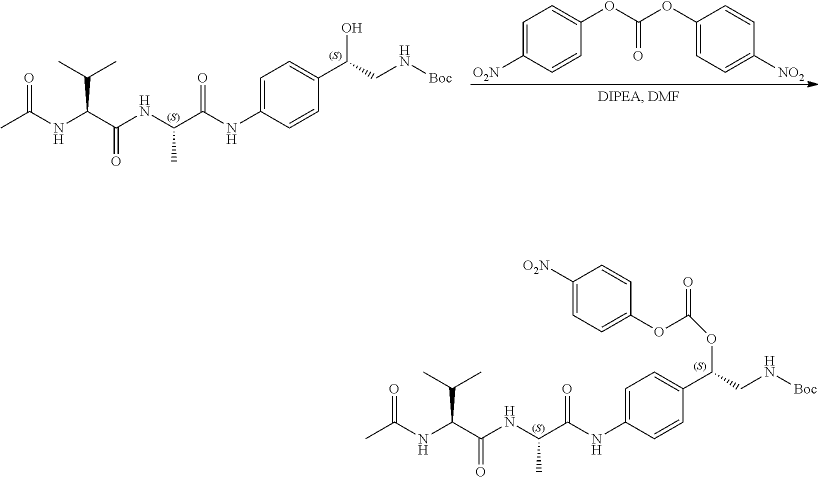

### tert-Butyl ((S)-2-(4-((S)-2-((S)-2-acetamido-3-methylbutanamido) propanamido)phenyl)-2-(((4-nitrophenoxy) carbonyl)oxy)ethyl) carbamate

tert-Butyl ((S)-2-(4-((S)-2-((S)-2-acetamido-3-methylbutanamido) propanamido)phenyl)-2-hydroxyethyl) carbamate (100.0 g, 215 mmol, 1 eq) was solubilized in anhydrous DMF (900 g) under N₂. Temperature was adjusted to 5 to 15° C. and stirred for 5 to 15 min. Bis(4-nitrophenyl) carbonate (132.0 g, 434 mmol, 2.0 eq) was added. DIPEA (83.5 g, 3 eq) was added dropwise at 5 to 15° C. under N₂. Temperature was adjusted to 15 to 25° C. and stirred 4 to 6 h. More bis(4-nitrophenyl) carbonate (6.5 g, 21 mmol, 0.10 eq) was added. Stirred 1 to 2 h at 15 to 25° C. under N₂. Added EtOAc (4400 g) and H₂O (4500 g). Stirred 10 to 30 min at 15 to 25° C. Allowed to stand for 10 to 30 min. Separated the organic layer and kept for later. Added EtOAc (4400 g) to the aqueous layer and stirred 10 to 30 min at 15 to 25° C. Allowed to stand for 10 to 30 min. Added the previous organic layer back in, followed by addition of 10% aqueous NaCl (4000 g). Stirred at 15 to 25° C. for 10 to 30 min. Allowed to stand for 10 to 30 min. Separated aqueous layer. Added anhydrous Na₂SO₄ (200 g) to the organic layer. Adjusted temperature to 15 to 25° C. and stirred for 10 to 30 min. Filtered and washed the filter cake with EtOAc (200 g). Collected the organic layer and concentrated below 40° C. to about half volume. Adjusted temperature to 30 to 40° C. and stirred for 10 to 30 min. Added n-heptane (1400 g) dropwise at 30 to 40° C. over 0.5 h. Adjusted temperature to 10 to 20° C. and stirred for 3 to 6 h. Filtered and washed filter cake with 1:2 EtOAc/n-heptane (500 g). Took up filter cake in EtOAc (9000 g) under N₂, adjusted temperature to 30 to 40° C., and stirred for 10 to 30 min. Added n-heptane (1400 g) dropwise at 30 to 40° C. over 0.5 h. Adjusted temperature to 10 to 20° C. and stirred for 3 to 6 h. Filtered and washed filter cake with 1:2 EtOAc/n-heptane (500 g). Dried filter cake at 30 to 40° C. for 20 to 30 h. Obtained title compound (119 g, 88%) as a solid.

### Step 6.4

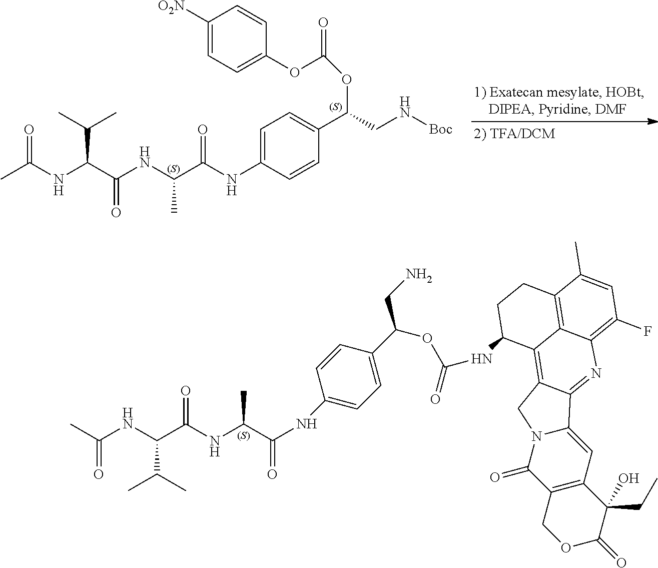

### (S)-1-(4-((S)-2-((S)-2-Acetamido-3-methylbutanamido) propanamido)phenyl)-2-aminoethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate

To a suspension of Exatecan mesylate (100.0 g, 188.1 mmol, 1 eq) in DMF (1504 g) and pyridine (392 g) at 10 to 20° C. under N₂ was added tert-butyl ((S)-2-(4-((S)-2-((S)-2-acetamido-3-methylbutanamido) propanamido)phenyl)-2-(((4-nitrophenoxy) carbonyl)oxy)ethyl) carbamate (142.2 g, 225.8 mmol, 1.2 eq), HOBt (25.42 g, 188.1 mmol, 1.0 eq), and DIPEA (48.63 g, 376.3 mmol, 2.0 eq). Stirred for 4 to 6 h under N₂ at 10 to 20° C. Adjusted temperature to 15 to 25° C. Added DCM (8000 g) and 10% aqueous Na₂CO₃ and stirred for 10 to 30 min. Allowed to stand for 10 to 30 min and separated the aqueous layer. Added DMF (450 g), H₂O (1000 g), and 10% aqueous citric acid (150.0 g) to the organic layer, adjusting pH 6 to 8. Stirred 5 to 10 min at 10 to 20° C. Allowed to stand for 5 to 30 min under N₂ and separated the aqueous layer. Added anhydrous Na₂SO₄ (500 g) to the organic layer and stirred at 10 to 20° C. for 10 to 30 min. Filtered and washed with DCM (500 g). Concentrated the filtrate at ≤30° C. This mixture was added dropwise to MTBE (3000 g) and adjusted temperature to 10 to 20° C. Stirred 0.5 to 1 h under N₂ at 10 to 20° C. Filtered mixture and washed with MTBE (800 g). Added DCM to the filter cake under N₂, adjusted temperature to −10 to 0° C., and stirred for 5 to 15 min. Added TFA (459 g) dropwise over 0.5 h. Stirred 1 to 4 h under N₂ at −10 to 0° C. Added more TFA (30 g) dropwise. Stirred 0.5 to 2 h under N₂ at −10 to 0° C. Added more TFA (30 g) dropwise. Stirred 0.5 to 2 h under N₂ at −10 to 0° C. Added reaction solution dropwise over 0.5 h to a solution of MTBE (14000 g) and n-heptane (3415 g) cooled to −10 to 5° C. Filtered reaction mixture and washed with MTBE (800 g). Dried the filter cake at 25 to 35° C. for 10 to 15 h. Obtained title compound (139 g, 90%) as a solid.

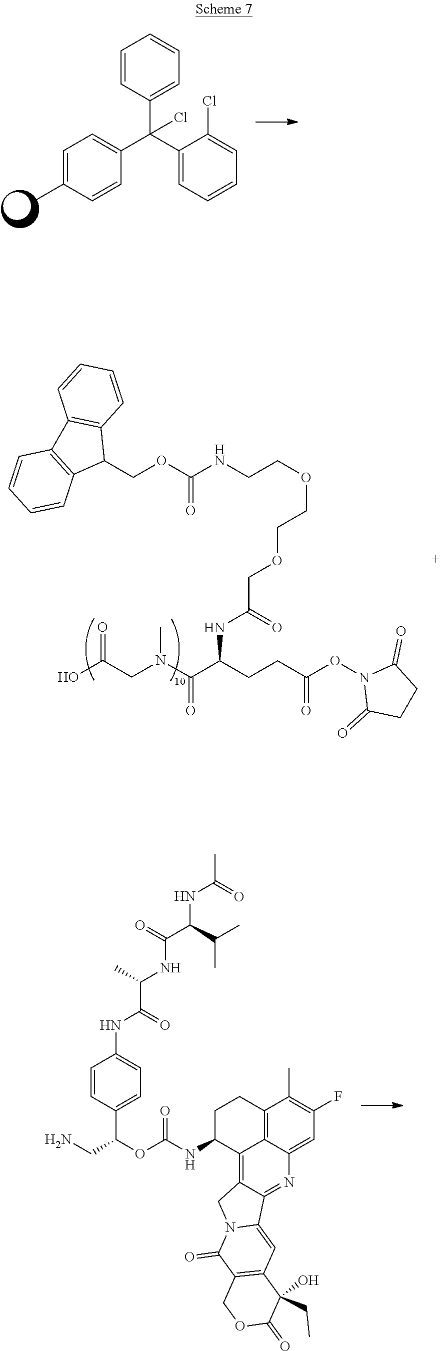

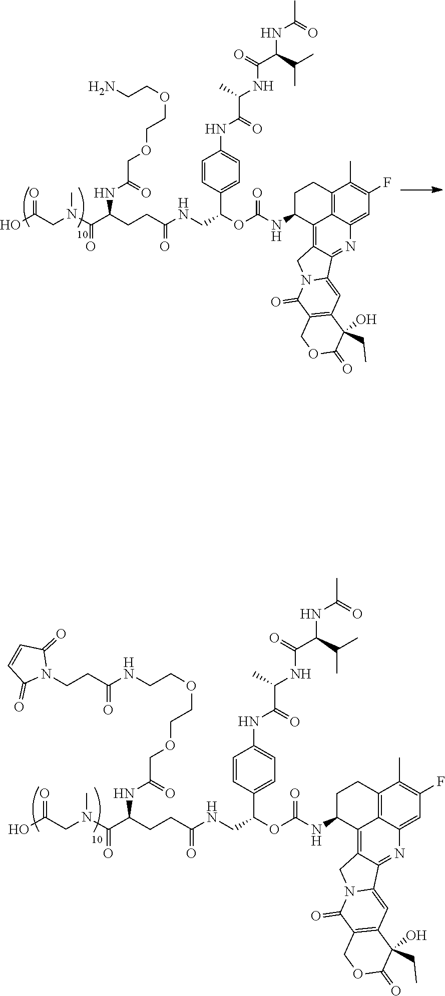

### Step 7.1

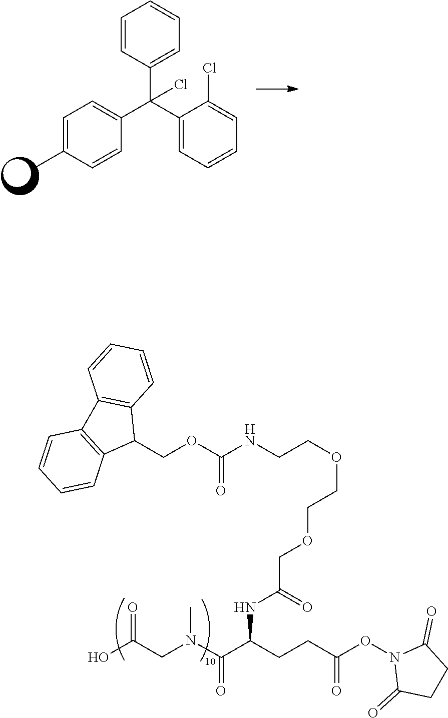

### (S)-14-(3-((2,5-dioxopyrrolidin-1-yl)oxy)-3-oxopropyl)-1-(9H-fluoren-9-yl)-16,19,22,25,28,31,34,37,40,43-decamethyl-3,12,15,18,21,24,27,30,33,36,39,42-dodecaoxo-2,7,10-trioxa-4,13,16,19,22,25,28,31,34,37,40,43-dodecaazapentatetracontan-45-oic acid

1) Added DCM to CTC Resin (12.80 g, 10.00 mmol, 0.78 mmol/g) and Fmoc-Sar-OH (2.48 g, 8.00 mmol, 0.80 eq) with N₂ bubbling.

2) Added DIPEA (4.00 eq) dropwise and mix for 2 h.

3) Added MeOH (13 mL) and mixed for 30 min.

4) Drained and washed with DMF 5 times.

5) Added 20% piperidine/DMF and reacted 30 min.

6) Drained and washed with DMF 5 times.

7) Added Fmoc-amino acid solution and mixed 30 seconds, then added activation buffer, N₂ bubbling for about 1 h.

8) Repeated above steps 4 to 7 for the coupling of following amino acids: Synthesized scale: 10.00 mmol

| No. | Materials | Coupling reagents |
|---|---|---|
| 1 | Fmoc-Sar-OH (0.80 eq) | DIPEA (4.00 eq) |
| 2 | Fmoc-Sar-OH (3.00 eq) | HBTU (2.85 eq) and DIPEA (6.00 eq) |
| 3 | Fmoc-Sar-OH (3.00 eq) | HBTU (2.85 eq) and DIPEA (6.00 eq) |
| 4 | Fmoc-Sar-OH (3.00 eq) | HBTU (2.85 eq) and DIPEA (6.00 eq) |
| 5 | Fmoc-Sar-OH (3.00 eq) | HBTU (2.85 eq) and DIPEA (6.00 eq) |
| 6 | Fmoc-Sar-OH (3.00 eq) | HBTU (2.85 eq) and DIPEA (6.00 eq) |
| 7 | Fmoc-Sar-OH (3.00 eq) | HBTU (2.85 eq) and DIPEA (6.00 eq) |
| 8 | Fmoc-Sar-OH (3.00 eq) | HBTU (2.85 eq) and DIPEA (6.00 eq) |
| 9 | Fmoc-Sar-OH (3.00 eq) | HBTU (2.85 eq) and DIPEA (6.00 eq) |
| 10 | Fmoc-Sar-OH (3.00 eq) | HBTU (2.85 eq) and DIPEA (6.00 eq) |
| 11 | Fmoc-Glu(OAll)-OH | HATU (2.85 eq) and DIPEA (6.00 eq) |
| | (3.00 eq) | |
| 12 | Fmoc-AEEA-OH | HATU (2.85 eq) and DIPEA (6.00 eq) |
| | (3.00 eq) | |
| 13 | De-Alloc | PhSiH₃(10.00 eq) and Pd(PPh₃)₄ |
| | | (0.10 eq) |
| 14 | NHS (60.00 eq) | DIC (50.00 eq) |

The coupling reaction was monitored by ninhydrin test, and the resin was washed with DMF 5 times.

### Peptide Cleavage and Purification:

1) Added cleavage buffer (1% TFA, 99% DCM) to the flask containing the side chain protected peptide at room temperature.

2) Filtered and collected the filtrate.

3) Removed the solvent under vacuum to give the crude peptide.

4) Purified the crude peptide by Prep-HPLC (A: 0.075% TFA in H₂O, B: ACN) to give the title compound (3.10 g, 90.80% purity, 23.44% yield).

### Purification Conditions:

Dissolution condition: Dissolved in ACN/H₂O
Instrument: Hanbon DAC-100
Mobile Phase: A: H₂O (0.1% TFA in H₂O); B: 0.1% TFA in ACN
Gradient 18-48%-42 min.
Retention time: 30 min
Column: Luna 250*100 mm, C18, 10 μm, 100 Å
Flow Rate: 250 mL/min
Wavelength: 220/254 nm
Oven Temp.: Room temperature

### Step 7.2

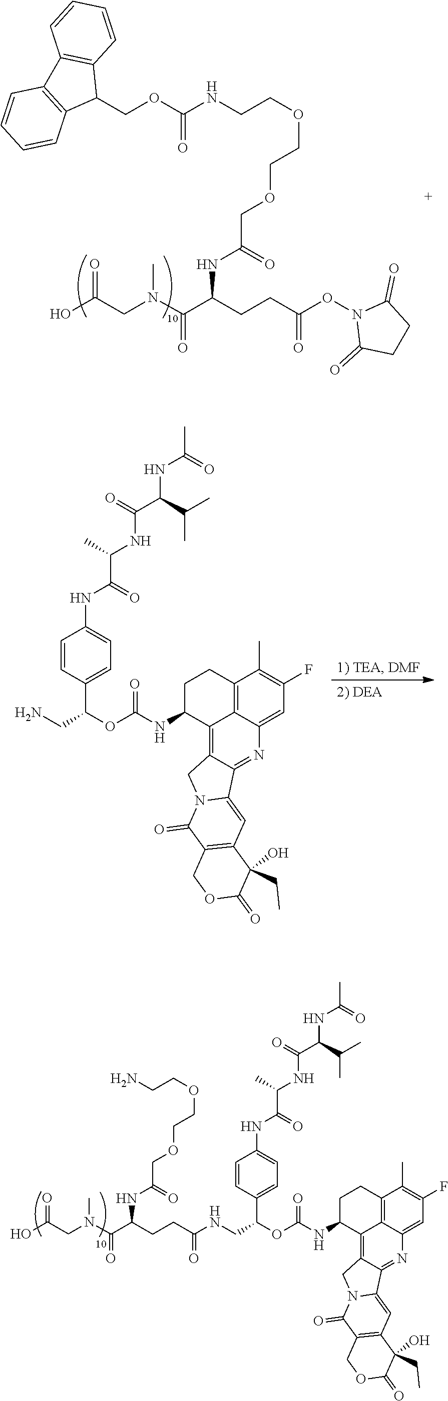

### (S)-32-(3-(((S)-2-(4-((S)-2-((S)-2-acetamido-3-methylbutanamido) propanamido)phenyl)-2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy)ethyl)amino)-3-oxopropyl)-41-amino-3,6,9,12,15,18,21,24,27,30-decamethyl-4,7,10,13,16,19,22,25,28,31,34-undecaoxo-36,39-dioxa-3,6,9,12,15,18,21,24,27,30,33-undecaazahentetracontanoic acid

Added(S)-1-(4-((S)-2-((S)-2-acetamido-3-methylbutanamido) propanamido)phenyl)-2-aminoethyl ((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl) carbamate (100.0 g, 1 eq) and DMF (400 g) to a mixture of(S)-14-(3-((2,5-dioxopyrrolidin-1-yl)oxy)-3-oxopropyl)-1-(9H-fluoren-9-yl)-16,19,22,25,28,31,34,37,40,43-decamethyl-3,12,15,18,21,24,27,30,33,36,39,42-dodecaoxo-2,7,10-trioxa-4,13,16,19,22,25,28,31,34,37,40,43-dodecaazapentatetracontan-45-oic acid (121.68 g) and DMF (540 g) cooled to −5 to 5° C. under N₂ and stirred for 5 to 15 min. TEA (49 g, 4.0 eq) was added over 15 min and stirred for 1 to 2 h under N₂ at −5 to 5° C. Added more(S)-1-(4-((S)-2-((S)-2-acetamido-3-methylbutanamido) propanamido)phenyl)-2-aminoethyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl) carbamate (1.0 g, 0.01 eq) and stirred 1 to 2 h. Added DEA (35.4 g) dropwise over 0.5 h. Stirred for 12 to 22 h under N₂ at −5 to 5° C. Transferred the reaction solution dropwise over 0.5 h to MTBE (14800 g) cooled to 0 to 10° C. and stirred 0.5 to 1 h under N₂. The reaction mixture was filtered and washed with MTBE (1200 g). The filter cake was dried at 5 to 15° C. for 5 to 12 h under N₂ flow. Obtained title compound (105.2 g, 48%) as a solid.

### Step 7.3

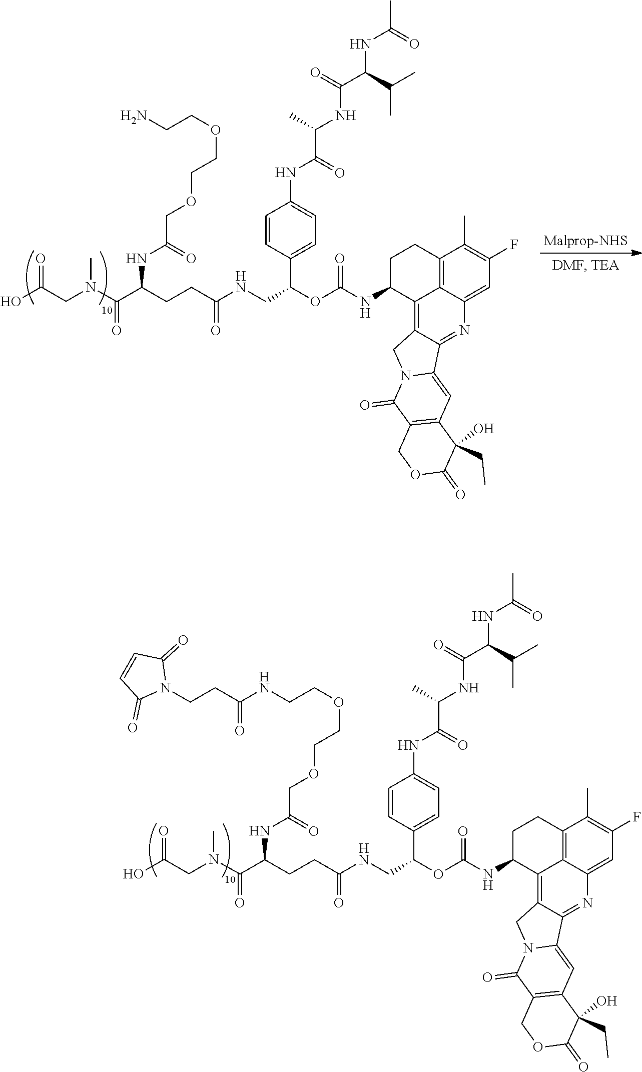

### (S)-32-(3-(((S)-2-(4-((S)-2-((S)-2-Acetamido-3-methylbutanamido) propanamido)phenyl)-2-((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl) carbamoyl)oxy)ethyl)amino)-3-oxopropyl)-45-(2,5-dioxo-2,5-dihydro-1H-pyrrol-1-yl)-3,6,9,12,15,18,21,24,27,30-decamethyl-4,7,10,13,16,19,22,25,28,31,34,43-dodecaoxo-36,39-dioxa-3,6,9,12,15,18,21,24,27,30,33,42-dodecaazapentatetracontanoic acid

Added 3-maleimidopropionic acid N-hydroxysuccinimide ester (16.8 g, 63.1 mmol) and DMF (100 g) to a solution of(S)-32-(3-(((S)-2-(4-((S)-2-((S)-2-acetamido-3-methylbutanamido) propanamido)phenyl)-2-((((1S,9S)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl) carbamoyl)oxy)ethyl)amino)-3-oxopropyl)-41-amino-3,6,9,12,15,18,21,24,27,30-decamethyl-4,7,10,13,16,19,22,25,28,31,34-undecaoxo-36,39-dioxa-3,6,9,12,15,18,21,24,27,30,33-undecaazahentetracontanoic acid (100.0 g, 55.2 mmol) and DMF (900 g) cooled to −15 to −5° C. Stirred 5 to 15 min under N₂ at −15 to −5° C. Added TEA (5.59 g) dropwise and stirred 1 to 4 h under N₂ at −15 to −5° C. Added more TEA (0.559 g) dropwise and stirred 0.5 to 2 h under N₂ at −15 to −5° C. Added MTBE (7400 g) and adjust temperature to −5 to 5° C. Filtered reaction mixture and washed with MTBE (600 g). Dried filter cake at 5 to 15° C. for 5 to 12 h under N₂ flow. Obtained title compound (73.6 g, 68%) as a solid.

Part I. Preparation of ADCs with Maleimide Linker-Payload

To prepare antibody-drug conjugates with eight drugs per antibody (DAR 8), the IgG1 antibody is fully reduced using a reducing reagent such as DTT or TCEP at 6 to 8 molar equivalents at 37° C. for 2 h. The reduced antibody is then buffer exchanged using a PD-10 desalting column with 50 mM HEPES with 2 mM EDTA at pH 7.0, and the eluent is adjusted with HEPES buffer to a protein concentration between 5-10 mg/ml. An excess of linker-payloads, such as 10 molar equivalents, is added for 1 hour, and the conjugation reaction may be stopped by adding a substantial excess of L-cysteine, such as 6 molar equivalents. The resulting mixture of ADCs may be purified on a PD-10 desalting column equilibrated in 25 mM histidine, 9% sucrose at pH 5.5, followed by 3 spin cycles with a 30 kDa MWCO centrifugal unit to remove any unreacted linker-payload related species. Finally, the resulting ADC may be sterile filtered through a 0.2-μM filter and stored at 4° C. or −80° C. for future use.

Part II. Preparation of ADCs with Bromoacetyl Linker-Payload

To prepare antibody-drug conjugates with eight drugs per antibody, the IgG1 antibody is fully reduced using a reducing reagent such as DTT or TCEP at 6 to 8 molar equivalents at 37° C. for 2 h. The reduced antibody is then buffer exchanged using a PD-10 desalting column with 50 mM HEPES with 2 mM EDTA at pH 7.4, and the eluent is adjusted with HEPES buffer to a protein concentration between 5-10 mg/ml. An excess of linker-payloads, such as 12 molar equivalents, is added for 2-3 hrs., and the conjugation reaction may be stopped by adding a substantial excess of L-cysteine, such as 10 molar equivalents. The resulting mixture of ADCs may be purified on a PD-10 desalting column equilibrated in 25 mM Histidine 9% Sucrose at pH 5.5, followed by 3 spin cycles with a 30 kDa MWCO centrifugal unit to remove any unreacted linker-payload related species. Finally, the resulting ADC may be sterile filtered through a 0.2 UM filter and stored at 4° C. or −80° C. for future use.

### Example 3: Antibody Binding Affinity, Cross-Reactivity, and Selectivity

### Characterizing the Human and Cross-Species Binding of PTK-7 ADCs by Surface Plasmon Resonance

A Biacore 8K+ instrument (Cytiva, Marlborough, MA) was used to determine kinetics and affinity parameters for the binding interactions of ADCs binding to recombinant human PTK7 (Acro Biosystems Cat. No. PT7-H52H3, Newark, DE).

An anti-human Fc sensor surface was prepared by amine-coupling of goat anti-human IgG Fc (Southern Biotech Cat. No. 2014-01, Birmingham, AL) to a Biacore Series S C1 (Cytiva Cat. No. BR-100535) sensor surface at 25° C. For immobilization, a running buffer of 10 mM HEPES, 150 mM NaCl, 0.05% Surfactant P20, pH 7.4 was used. Flow cells 1 & 2 of all 8 channels were activated with a 1:1 (v/v) mixture of 400 mM 1-Ethyl-3-(3-dimethylaminopropyl) carbodiimide hydrochloride (EDC) and 100 mM N-Hydroxysuccinimide (NHS) at a flow rate of 10 μL/min for 7 minutes. Then the anti-human IgG Fc capture reagent was coupled to the sensor surface (diluted to 50 μg/mL in 10 mM Acetate pH 4.5 buffer) by injecting it in all flow cells and channels at a flow rate of 10 μL/min for 7 minutes. Remaining active groups were blocked by injecting 100 mM ethylenediamine (in 200 mM Borate buffer, pH 8.5) in all flow cells and channels at a flow rate of 10 μL/min for 7 minutes. All channels and flow cells were then preconditioned using three consecutive 1-minute injections of 75 mM phosphoric acid at 10 L/min.

For kinetics and affinity analysis, the running and sample dilution buffer was 10 mM HEPES, 150 mM NaCl, 0.05% Surfactant P20, pH 7.4, 1 mg/mL bovine serum albumin (BSA) and the analysis temperature was 37° C.

In each analysis cycle, a different ADC was captured on flow cell 2 of each channel by injecting a 5 μg/mL solution at 10 μL/min for 2 minutes. After capture, the same analyte was injected on flow cells 1 and 2 in all 8 channels for 2 minutes at 30 μL/min and dissociation was monitored for 10 minutes. After dissociation, all surfaces were regenerated with three consecutive 1-minute injections of 75 mM phosphoric acid at 10 μL/min. ADC capture and analyte cycles were repeated to obtain human PTK7 analyte binding of each ADC at analyte concentrations of 0, 2.47, 7.41, 22.2, 66.7, 200, and 600 nMs.

Sensorgram data were reference subtracted and blank subtracted, then globally fit with the default 1:1 binding model in Biacore Insight Evaluation Software v3.0.12.15655, as shown in Table 4.

Certain antibodies of the present disclosure, including Abs 1-4, bind cynomolgus monkey with K_{D}'s in the nM range.

TABLE 4
Kinetics and affinity parameters for
ADC/PTK-7 interactions at 37° C.

| Ab in the ADC | Antigen | kₐ (1/Ms) | k_{d} (1/s) | K_{D} (nM) |
|---|---|---|---|---|
| Ab1-PSAR10- | human PTK-7 | 1.19 × 10⁶ | 3.42 × 10⁻² | 28.9 |
| VA-exatecan | | | | |
| Ab2- PSAR10- | human PTK-7 | 1.81 × 10⁵ | 1.34 × 10⁻² | 74.2 |
| VA-exatecan | | | | |
| Ab3- PSAR10- | human PTK-7 | 1.30 × 10⁵ | 8.04 × 10⁻³ | 61.8 |
| VA-exatecan | | | | |
| Ab4- PSAR10- | human PTK-7 | 3.68 × 10⁵ | 3.72 × 10⁻² | 101 |
| VA-exatecan | | | | |

Kinetics and affinity parameters for ADC/human PTK7 interactions at 37° C. kₐ is the association rate constant, k_{d} is the dissociation rate constant, K_{D} is the equilibrium dissociation constant (calculated using K_{D} = k_{d}/kₐ).

### Characterizing Cell Surface Binding of PTK-7 Antibodies on Cell Lines Expressing the PTK-7 Receptor

Certain PTK-7 antibodies of the present disclosure were tested for cell surface binding to three PTK-7 expressing tumor cell lines. Cell lines were selected to represent high, medium, and low receptor densities. OV90-PTK-7, overexpressing engineered ovarian tumor line representing high expression, NCI-H446 human lung cancer cells representing medium endogenous expression and OVCAR-3 human ovarian cancer cells representing low endogenous expression. OV90-PTK-7, NCI-H446 and OVCAR3 cells were determined to have antibody binding capacity of ˜500,000, ˜40,000 and ˜20,000 respectively (using MESF quantification kit, Bangs Laboratories). OV90 parental cells and Namalwa-luc-GFP cells were selected as the PTK-7 negative cell lines. Antibody binding was quantified via flow cytometry and both the EC₅₀ of the binding curves and the maximum binding MFI for each antibody were recorded.

Cells were dissociated using non-enzymatic dissociation buffer for 5 min at 37° C. Cells were counted and aliquoted to a V-bottom polypropylene 96-well plate at 10⁵ cells/well. Cells were centrifuged at 1800 rpm×3 min and the supernatant discarded. An 11-point antibody dilution series was prepared in assay buffer (1×PBS containing 5% FBS) starting at 300 nM and diluting 1:4 down. The dilution series was added to cells at 100 μL/well and mixed by pipetting. Several untreated control wells/cell lines were prepared in assay buffer alone. Cells and antibodies were incubated at 4° C. on an orbital shaker for 1 hour. After incubation, the assay plates were centrifuged and washed twice with 300 μL/well of assay buffer. Cell pellets were then stained with a 1:500 dilution of Alexa647 conjugated mouse anti-human IgG secondary antibody in assay buffer, at 100 μL/well. Assay plates were incubated shaking in the dark at 4° C. for 1 hour. After incubation, plates were centrifuged, and the cells were washed twice with 300 μL/well of assay buffer. A 1:2000 dilution of Zombie Green viability marker in 1×PBS was aliquoted to the cells at 100 μL/well and plates were incubated in the dark for 10 min shaking at room temperature. Cell pellets were then washed 1× with assay buffer and fixed with 200 μL/well of 4% paraformaldehyde in 1×PBS in the dark at room temperature for 15 min. Cells were centrifuged and washed before resuspending in 100 μL of assay buffer for acquisition on Attune Cytpix Cytometer.

Cells were acquired on an Attune Cytpix Cytometer and FCS files generated using Attune™ Cytometric Software (v. 6.0.1). FCS files were then analyzed on FlowJo (v10.8.1). Debris was excluded from analysis by Forward scatter (FSC) vs side scatter (SSC) gating and single cells were selected via forward scatter area (FSC-A) vs height (FSC-H) gating. Finally, dead cells positive for staining with Zombie Green were excluded and the MFIs of live, Alexa647 positive cells were quantified. Data was graphed and analyzed on GraphPad Prism (v10.1.1). EC₅₀s were determined via agonist vs response—variable slope (four-parameter) curve fitting and % of Cofetuzumab maximum binding calculated by setting the average of maximum Cofetuzumab MFIs for each cell line at 100%.

Certain PTK7 antibodies of the present disclosure did not bind to PTK-7 negative cell lines OV90 parental and namalwa-luc-GFP, as expected. The four antibodies shown in Table 5 bound to all three PTK-7 expressing tumor lines.

TABLE 5
Cell Surface Binding of PTK-7 Antibodies to PTK-7 expressing cell lines.
Max MFI, Percent of Cofetuzumab Max Binding of PTK-7 Antibodies

| | | | | | % of Cofetuzumab | | | | | |
|---|---|---|---|---|---|---|---|---|---|---|
| | | Average Max MFI | | | (Average Max MFI) | | | EC₅₀ (nM) | | |
| | Cell | OV90- | NCI- | | OV90- | NCI- | | OV90- | NCI- | |
| mAbs | lines | PTK-7 | H446 | OVCAR3 | PTK-7 | H446 | OVCAR3 | PTK-7 | H446 | OVCAR3 |
| Cofetuzumab | | 6.85E+04 | 2.68E+04 | 2.19E+04 | 100% | 100% | 100% | 1.41 | 0.54 | 0.70 |
| Ab1 | | 6.61E+04 | 2.32E+04 | 1.57E+04 | 96.5% | 86.3% | 71.6% | 1.25 | 0.70 | 2.81 |
| Ab2 | | 7.05E+04 | 2.62E+04 | 1.92E+04 | 103% | 97.6% | 87.7% | 1.83 | 0.60 | 0.52 |
| Ab3 | | 7.12E+04 | 2.79E+04 | 2.02E+04 | 104% | 104% | 92% | 1.35 | 0.52 | 0.41 |
| Ab4 | | 6.99E+04 | 2.61E+04 | 1.64E+04 | 102% | 97.4% | 75% | 1.28 | 0.54 | 0.95 |

MFI = Median Fluorescence Intensity

### Example 4: ADC Binding and Internalization

### Characterizing the Internalization Capacity of PTK-7 Antibodies and ADCs in Human PTK-7 Positive Cells Using Fluorescence Imaging

Single clone HeLa PTK-7-eGFP overexpressing engineered cells were plated at 5,000 cells per well in a 384-well plate (PhenoPlate 384-well, black, optically clear flat-bottom, tissue-culture treated, PerkinElmer 6057308) in complete culture medium (DMEM, +10% FBS+Glutamax+Pen/Strep). Next day, the cells were treated with 6 μg/ml (40 nM) ADCs or antibodies and pHrodo labeling reagent (Zenon™ pHrodo™ iFL IgG labeling reagents, Catalog number: Z25612, Invitrogen™) with concentration of 6 μg/mL (120 nM). The ADCs or antibodies was mixed with pHrodo labeling reagent for at least 10 min prior to adding to the cells. Then, the cells were imaged using the Opera Phenix Plus high-content screening system every 90 min up to 24 hours. Data were processed and analyzed in Harmony and Microsoft Excel and graphed in GraphPad Prism.

As shown in Table 6, PTK-7 antibodies and PTK-7 ADCs exhibit superior lysosomal trafficking (approximately double) in comparison to cofetuzumab pelidotin.

TABLE 6
Percent activity and EC₅₀ (nM) of PTK-7 Antibodies and ADCs

| | pHrodo intensity | | pHrodo intensity |
|---|---|---|---|
| Antibodies | signal at 24 hrs | ADCs | signal at 24 hrs |
| Cofetuzumab | 2.44 ± 0.14 | Cofetuzumab- | 1.71 ± 0.06 |
| | | Pelidotin | |
| Ab1 | 3.00 ± 0.1 | Ab1-PSAR10- | 3.72 ± 0.23 |
| | | VA-Exatecan | |
| Ab2 | 3.31 ± 0.08 | Ab2-PSAR10- | 3.67 ± 0.15 |
| | | VA-Exatecan | |
| Ab3 | 2.64 ± 0.1 | Ab3-PSAR10- | 3.56 ± 0.18 |
| | | VA-Exatecan | |
| Ab4 | 2.83 ± 0.18 | Ab4-PSAR10- | 3.99 ± 0.29 |
| | | VA-Exatecan | |

### Example 5: ADC Cytotoxicity and Bystander Activity

### Characterizing the Activity of PTK-7 ADCs in Low (OVCAR-3, ABC=˜20,000) and High (OV90-PTK-7, ABC=˜500,000) PTK-7 Expressing Cell Lines

OVCAR3 cells were seeded in white clear-bottom 96-well tissue culture plates in culture medium (OVCAR3: ATCC modified RPMI 1640+1× GlutaMax+20% Heat Inactivated Fetal Bovine Serum+10 μg/mL bovine insulin). Cells were incubated at 37° C. with 5% CO₂ overnight. Next day, ADCs were added at final working concentration from 200 nM, 1:4 serially diluted in culture medium. Plates were covered with Breathe-Easy® sealing membrane and incubated at 37° C. with 5% CO₂. Plates were read after 7 days treatment using CellTiter-Glo Luminescent Cell Viability Assay. 100 μL/well CellTiter-Glo reagent were incubated in plates at room temperature for 10 min. Luminescence was read in the SpectraMax M5e. RLUs (Relative Light Units) were obtained with SoftMax Pro 5.4. Percentage of cell killing was calculated against no treatment as 0%. Data was graphed and analyzed using Graphpad Prism version v10.1.1. IC₅₀ was determined via log (inhibitor) vs. response-variable slope (four parameters) curve fitting.

OV90-PTK-7 engineered cell line was seeded in culture medium (1:1 MCDB105: Medium 199+15% Heat Inactivated Fetal Bovine Serum+1×Glutamax). ADCs were added at final working concentration from 200 nM, 1:4 serially diluted in culture medium for 5 days.

Certain PTK-7 ADCs of the present disclosure exhibited similar robust maximal cell killing as Cofetuzumab-pelidotin conjugate on PTK-7 cell lines with differential expression levels. IC₅₀ values suggest similar potencies across all ADCs in OV90-PTK-7 cells and variable potencies amongst ADCs tested in OVCAR3 cells. No non-specific cytotoxicity was observed on OV90 Parental (PTK-7 negative cell line).

TABLE 7
Cytotoxicity of PTK-7 exatecan conjugates
on high and low expression PTK-7 cell lines

| | OV90-PTK-7 | OVCAR-3 |
|---|---|---|
| ADCs | (IC₅₀; nM) | (IC₅₀; nM) |
| Ab1-VA-Exatecan | 4.49 e−008 | 4.00 |
| Ab2-VA-Exatecan | 2.06 e−008 | 4.84 |
| Ab3-VA-Exatecan | 4.88 e−009 | 1.12 |
| Ab4-VA-Exatecan | 8.0 e−008 | 2.02 |
| Cofetuzumab-Pelidotin | 3.31e−005 | 2.53 |

Characterizing Bystander Effect of PTK-7 ADCs in Namalwa-Luc-GFP Cells (PTK-7 Negative Cell-Line) Co-Cultured with OV90-PTK-7 and NCI-H446 (PTK-7 Positive Cell Lines with Different Expression Levels)

In flat clear bottom 96-well plates, OV90-PTK-7: Namalwa-Luc-GFP clone cells were mixed and seeded at a total of 3000 cells/well/100 μL with a ratio of 2:1 in culture medium (1:1 MCDB105: Medium 199+15% Heat Inactivated Fetal Bovine Serum+1×Glutamax). Namalwa-Luc-GFP cells were negative for PTK-7 expression. Plates were incubated at 37° C. with 5% CO₂ overnight. Next day, ADCs were added at final working concentration from 50 nM, 1:4 serially diluted in culture medium. Plates were covered with Breathe-Easy® sealing membrane. Plates were read after 5-days treatment using ONE-Glo™ Luciferase Assay System. 100 μL/well ONE-Glo™ Assay reagent was incubated in plates at room temperature for 10 min. Luminescence was read in the SpectraMax M5e. RLUs (Relative Light Units) were obtained with SoftMax Pro 5.4. Percentage of Namalwa-Luc-GFP killing was calculated against no treatment as 0%. Data was graphed and analyzed using Graphpad Prism version v10.1.1. IC₅₀ was determined via log (inhibitor) vs. response (three parameters) curve fitting for ADC cytotoxicity.

In flat clear bottom 96-well plates, NCI-H446: Namalwa-Luc-GFP clone cells were mixed and seeded at a total of 5000 cells/well/100 μL with a ratio of 9:1 in culture medium (RPMI 1640+10% Heat Inactivated Fetal Bovine Serum+1× Glutamax+1 mM sodium pyruvate). Plates were incubated at 37° C. with 5% CO₂ overnight. Next day, ADCs were added at final working concentration from 300 nM, 1:4 serially diluted in culture medium. Plates were covered with Breathe-Easy® sealing membrane. Plates were read after 7-days treatment using ONE-Glo™ Luciferase Assay System.

As shown in Table 8, certain PTK-7 ADCs of the present disclosure showed better bystander effect than cofetuzumab pelidotin on Namalwa-Luc-GFP cells with OV90-PTK-7 as positive cell line and similar bystander effect on Namalwa-Luc-GFP cell with NCI-H446 as positive cell line.

TABLE 8
IC₅₀s of Bystander Effect by PTK-7 Exatecan
conjugates on Namalwa-Luc-GFP negative cells

| | OV90-PTK-7 + | NCI-H446 + |
|---|---|---|
| | Namalwa-Luc-GFP | Namalwa-Luc-GFP |
| ADCs | (IC₅₀; nM) | (IC₅₀; nM) |
| Ab1-VA-Exatecan | 0.51 | 3.71 |
| Ab2-VA-Exatecan | 0.58 | 4.47 |
| Ab3-VA-Exatecan | 0.54 | 6.03 |
| Ab4-VA-Exatecan | 0.48 | 4.51 |
| Cofetuzumab-Pelidotin | 1.50 | 3.18 |

### Example 6: ADCC, ADCP, and/or CDC Assays

### In Vitro Antibody-Dependent Cell-Mediated Cytotoxicity (ADCC) Assay for PTK-7 Antibodies

OV90 cell line was engineered to express high level of human PTK-7, having antibody binding capacity of 500,000 (using MESF quantification kit, Bangs Laboratories). Target cells, OV90-human PTK-7, were added in clear tissue culture 96-well plates in test medium (IMDM+1× GlutaMax+10% Heat Inactivated Fetal Bovine Serum+Pen-Strep 100 U/ml-100 μg/mL) and incubated at 37° C. with 5% CO₂ overnight. Next day, 40 μL/well antibodies were added at final working concentration from 200 nM, 1:3 serially diluted in test medium. Antibodies were incubated at 37° C. with 5% CO₂ for one hour. Effector cells, Jurkat-Lucia NFAT-CD16, were then added at 150,000/80 μL/well and incubated at 37° C. with 5% CO₂. 24 h later, 20 μL supernatants and 50 μL of pre-prepared QUANTI-Luc/well were mixed in white opaque plates. Luminescence was read on a SpectraMax M5e. RLUs (Relative Light Units) were obtained with SoftMax Pro 5.4 and plotted on Y-axis against compound concentrations on X-axis using GraphPad Prism version 9.5.1.

Unlike cofetuzumab antibody, which has wildtype IgG1 Fc, PTK7 Abs 1˜4 of the present disclosure are effector null antibodies and did not result in antibody dependent cellular cytotoxicity potential.

### In Vitro Antibody-Dependent Cellular Phagocytosis (ADCP) Assay for PTK-7 Antibodies

OV90 cell line was engineered to express high level of human PTK-7, having antibody binding capacity of 500,000 (using MESF quantification kit, Bangs Laboratories). Target cells, OV90-human PTK-7, were seeded in clear tissue culture 96-well plates in test medium (IMDM+1× GlutaMax+10% Heat Inactivated Fetal Bovine Serum+Pen-Strep 100 U/ml-100 μg/mL) and incubated at 37° C. with 5% CO₂ overnight. Next day, 40 μL/well mAbs were added at final working concentration from 200 nM, 1:3 serially diluted in test medium and incubated at 37° C. with 5% CO₂ for one hour. Effector cells, Jurkat-Lucia NFAT-CD32, were then added at 200,000/80 μL/well and incubated at 37° C. with 5% CO₂. 24 h later, 20 μL supernatants and 50 μL of pre-prepared QUANTI-Luc/well were mixed in white opaque plates. Luminescence was read on a SpectraMax M5e. RLUs (Relative Light Units) were obtained with SoftMax Pro 5.4 and plotted on Y-axis against compound concentrations on X-axis using GraphPad Prism version 9.5.1.

Unlike cofetuzumab antibody, which has wildtype IgG1 Fc, the PTK-7 Abs 1˜4 of the present disclosure are effector null antibodies and did not result in antibody dependent cellular phagocytosis potential.

### In Vitro Complement-Dependent Cytotoxicity (CDC) Assay for PTK-7 Antibodies

OV90 cell line was engineered to express high level of human PTK7, having antibody binding capacity of 500,000 (using MESF quantification kit, Bangs Laboratories). OV90-human PTK7 cells were added in white clear-bottom 96-well tissue culture plates in assay medium (1:1 MCDB105/Medium199+1×GlutaMax+15% Heat Inactivated Fetal Bovine Serum) and incubated in the incubator at 37° C. with 5% CO₂ overnight. Next day, 50 μL/well antibodies were added at final working concentration from 200 nM, 1:3 serially diluted in assay medium and incubated at 37° C. with 5% CO₂ for one hour. Diluted human serum complement (1:3) was then added at 50 μL/well in assay medium and incubated at 37° C. with 5% CO₂ for 3 hours. Plate was read using CellTiter-Glo Luminescent Cell Viability Assay. 100 μL/well CellTiter-Glo reagent were incubated in plates at room temperature for 10 min. Luminescence was read on a SpectraMax M5e. RLUs (Relative Light Units) were obtained with SoftMax Pro 5.4. Percentage of cell killing was calculated against no treatment. Data was graphed and analyzed using GraphPad Prism version 9.5.1.

As a positive control, Jeko-1 cells were treated as above with anti-CD20 antibody in assay medium (RPMI1640+1×GlutaMax+10% Heat Inactivated Fetal Bovine Serum).

Like cofetuzumab, the PTK-7 Abs 1˜4 of the present disclosure did not show CDC potential in PTK-7 expressing OV90 cell line. Anti-CD20 control antibody resulted in CDC activity, used as positive control on Jeko-1 cells expressing CD20.

### Example 7: Efficacy of PTK-7 ADCs in Tumor Xenograft Models

Female athymic nude mice from Envigo between 5-7 weeks of age with weight 18-20 grams were subcutaneously implanted on the right flank with 5×10\hat{ }6 OVCAR3, NCI-H446 or OV90-PTK7 cells with 50% Matrigel in Hank's Balanced Salt Solution. When tumors reached approximately 150-250 mm³; animals were matched by tumor volume into treatment or control groups and dosing initiated (Day 0, n=5-8 per group). The test articles were given intravenously at a single dose (2 mg/kg for OVCAR3 and NCI-H446; 0.5 mg/kg for OV90-PTK7) formulated with 5% Dextrose. Tumors were measured biweekly using a digital caliper.

As shown in Table 8, treatment with the PTK-7 ADCs using Abs1-4 resulted in tumor growth inhibition in OVCAR3 cell line that was more efficacious than the benchmark cofetuzumab-pelidotin ADC.

TABLE 8
Tumor measurements (Mean tumor volume ± SEM)
after treatment with PTK-7 ADCs

| | OVCAR3 (Day 61) | | H446 (Day 32) | | OV90-PTK7 (Day 28) | |
|---|---|---|---|---|---|---|
| | Mean | | Mean | | Mean | |
| | (mm³) | SEM | (mm³) | SEM | (mm³) | SEM |
| Vehicle | 733.54 | 118.4 | 1090.86 | 239.6 | 1251.16 | 127.2 |
| Cofetuzumab | 352.26 | 57.2 | N/A | N/A | N/A | N/A |
| pelidotin | | | | | | |
| Ab1-PSAR10-VA- | 81.04 | 21.1 | 85.58 | 12.9 | 166.65 | 43.4 |
| Exatecan | | | | | | |
| Ab2-PSAR10-VA- | 101.24 | 7.4 | 139.31 | 31.1 | 138.26 | 8.3 |
| Exatecan | | | | | | |
| Ab3-PSAR10-VA- | 62.24 | 25.3 | 190.12 | 45.3 | 116.28 | 21.6 |
| Exatecan | | | | | | |
| Ab4-PSAR10-VA- | 59.01 | 21.5 | 128.18 | 27.1 | 182.01 | 9.4 |
| Exatecan | | | | | | |

SEM = Standard error of mean

### Example 8: Stability and Tolerability

Stability of an ADC with a PTK7 antibody of the present disclosure with PSAR10-VA-exatecan was assessed in rat and human plasma (100 μg/ml). Minimal DAR lost (˜2) over 7 days.

Tolerability of an ADC of the present disclosure with a PTK7 antibody of the present disclosure (with PSAR10-VA-exatecan) was tested in two male cynomolgus monkeys. The cynomolgus monkeys received 50 mg/kg by slow bolus IV injection on days 1 and 22. Samples were collected pre-dose and at ˜0.5 (30 mins), 1, 4, 8, 24, 48, 96, and 168, as well as (on day 1 only) 240, 336, and 504 hours post dose. Bioanalysis was done by LCMS using qualified methods. No ADA was detected. No mortalities, hematopoietic (BM, thymus, spleen, hematology), GI (min to moderate histopathology in GI tract). T1/2 was ˜8 days.

Amino Acid and Nucleotide Sequences

SEQ ID NO: 1 (human PTK-7)
MGAARGSPARPRRLPLLSVLLLPLLGGTQTAIVFIKQPSSQDALQGRRALLRCEVE
APGPVHVYWLLDGAPVQDTERRFAQGSSLSFAAVDRLQDSGTFQCVARDDVTG
EEARSANASFNIKWIEAGPVVLKHPASEAEIQPQTQVTLRCHIDGHPRPTYQWFR
DGTPLSDGQSNHTVSSKERNLTLRPAGPEHSGLYSCCAHSAFGQACSSQNFTLSIA
DESFARVVLAPQDVVVARYEEAMFHCQFSAQPPPSLQWLFEDETPITNRSRPPHL
RRATVFANGSLLLTQVRPRNAGIYRCIGQGQRGPPIILEATLHLAEIEDMPLFEPRV
FTAGSEERVTCLPPKGLPEPSVWWEHAGVRLPTHGRVYQKGHELVLANIAESDA
GVYTCHAANLAGQRRQDVNITVATVPSWLKKPQDSQLEEGKPGYLDCLTQATP
KPTVVWYRNQMLISEDSRFEVFKNGTLRINSVEVYDGTWYRCMSSTPAGSIEAQ
ARVQVLEKLKFTPPPQPQQCMEFDKEATVPCSATGREKPTIKWERADGSSLPEW
VTDNAGTLHFARVTRDDAGNYTCIASNGPQGQIRAHVQLTVAVFITFKVEPERTT
VYQGHTALLQCEAQGDPKPLIQWKGKDRILDPTKLGPRMHIFQNGSLVIHDVAP
EDSGRYTCIAGNSCNIKHTEAPLYVVDKPVPEESEGPGSPPPYKMIQTIGLSVGAA
VAYIIAVLGLMFYCKKRCKAKRLQKQPEGEEPEMECLNGGPLQNGQPSAEIQEE
VALTSLGSGPAATNKRHSTSDKMHFPRSSLQPITTLGKSEFGEVFLAKAQGLEEG
VAETLVLVKSLQSKDEQQQLDFRRELEMFGKLNHANVVRLLGLCREAEPHYMV
LEYVDLGDLKQFLRISKSKDEKLKSQPLSTKQKVALCTQVALGMEHLSNNRFVH
KDLAARNCLVSAQRQVKVSALGLSKDVYNSEYYHFRQAWVPLRWMSPEAILEG
DFSTKSDVWAFGVLMWEVFTHGEMPHGGQADDEVLADLQAGKARLPQPEGCP
SKLYRLMQRCWALSPKDRPSFSEIASALGDSTVDSKP
SEQ ID NO: 2 (HC of Ab1)
EVQLVESGGGLVKPGGSLRLSCAASGFTFSSYIMNWVRQAPGKGLEWVSSISSSS
TFIYYADSVKGRFTISRDNAKNSLYLQMNSLRAEDTAVYYCAKGFDYWGQGTL
VTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDK
THTCPPCPAPEAAGGPSVFLFPPKPKDTLMISRTPEVTCVVVSVSHEDPEVKFNW
YVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQ
KSLSLSPGK
SEQ ID NO: 3 (LC of Ab1)
DIQLTQSPSFLSASVGDRVTITCRASQDISSYLVWYQQKPGKAPKLLIYAASTLQS
GVPSRFSGSGSGTEFTLTISSLQPEDFATYYCQQLNTYPRTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO: 4 (HCDR1 of Ab1)
AASGFTFSSYIMN
SEQ ID NO: 5 (HCDR2 of Ab1)
SISSSSTFIY
SEQ ID NO: 6 (HCDR3 of Ab1)
AKGFDY
SEQ ID NO: 7 (LCDR1 of Ab1)
RASQDISSYLV
SEQ ID NO: 8 (LCDR2 of Ab1)
YAASTLQS
SEQ ID NO: 9 (LCDR3 of Ab1)
QQLNTYPRT
SEQ ID NO: 10 (VH of Ab1)
EVQLVESGGGLVKPGGSLRLSCAASGFTFSSYIMNWVRQAPGKGLEWVSSISSSS
TFIYYADSVKGRFTISRDNAKNSLYLQMNSLRAEDTAVYYCAKGFDYWGQGTL
VTVSS
SEQ ID NO: 11 (VL of Ab1)
DIQLTQSPSFLSASVGDRVTITCRASQDISSYLVWYQQKPGKAPKLLIYAASTLQS
GVPSRFSGSGSGTEFTLTISSLQPEDFATYYCQQLNTYPRTFGQGTKVEIK
SEQ ID NO: 12 (HC of Ab2)
QVQLQESGPGLVKPSETLSLTCTVSGGSISTYYWSWIRQPAGKGLEWIGRIYTSGL
TNYNPSLKSRVTMSVDTSKNQFSLKLSSVTAADTAVYYCARDPHYYDGSGFDY
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVE
PKSCDKTHTCPPCPAPEAAGGPSVFLFPPKPKDTLMISRTPEVTCVVVSVSHEDPE
VKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALH
NHYTQKSLSLSPGK
SEQ ID NO: 13 (LC of Ab2)
EIVLTQSPGTLSLSPGERATLSCRASQSVSDNYLAWYQQKPGQAPRLLIYGASSR
ATGIPDRFSGSGSGTDFTLTISRLEPEDFAVYYCQQYGSSPFTFGPGTKVDIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 14 (HCDR1 of Ab2)
TVSGGSISTYYWS
SEQ ID NO: 15 (HCDR2 of Ab2)
RIYTSGLTN
SEQ ID NO: 16 (HCDR3 of Ab2)
ARDPHYYDGSGFDY
SEQ ID NO: 17 (LCDR1 of Ab2)
RASQSVSDNYLA
SEQ ID NO: 18 (LCDR2 of Ab2 and Ab4)
YGASSRAT
SEQ ID NO: 19 (LCDR3 of Ab2)
QQYGSSPFT
SEQ ID NO: 20 (VH of Ab2)
QVQLQESGPGLVKPSETLSLTCTVSGGSISTYYWSWIRQPAGKGLEWIGRIYTSGL
TNYNPSLKSRVTMSVDTSKNQFSLKLSSVTAADTAVYYCARDPHYYDGSGFDY
WGQGTLVTVSS
SEQ ID NO: 21 (VL of Ab2)
EIVLTQSPGTLSLSPGERATLSCRASQSVSDNYLAWYQQKPGQAPRLLIYGASSR
ATGIPDRFSGSGSGTDFTLTISRLEPEDFAVYYCQQYGSSPFTFGPGTKVDIK
SEQ ID NO: 22 (HC of Ab3)
QVQLQESGPGLVKPSETLSLTCTVSGGSISTYYWNWIRQPAGKGLEWIGRIYSSGS
TNYNPSLKSRVTMSVDTSKNQFSLKLSSVTAADTAVYYCARDPLYSGSLFDIWG
QGTMVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKS
CDKTHTCPPCPAPEAAGGPSVFLFPPKPKDTLMISRTPEVTCVVVSVSHEDPEVKF
NWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHY
TQKSLSLSPGK
SEQ ID NO: 23 (LC of Ab3)
EIVLTQSPGTLSLSPGERATLSCRASQSVSSSYLAWYQQKPGQAPRLLIYGASIRA
TGIPDRFSGSGSGTDFTLTISRLEPEDFAVYYCQQYGSSPYTFGQGTKLEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 24 (HCDR1 of Ab3)
TVSGGSISTYYWN
SEQ ID NO: 25 (HCDR2 of Ab3)
RIYSSGSTN
SEQ ID NO: 26 (HCDR3 of Ab3)
ARDPLYSGSLFDI
SEQ ID NO: 27 (LCDR1 of Ab3)
RASQSVSSSYLA
SEQ ID NO: 28 (LCDR2 of Ab3)
YGASIRAT
SEQ ID NO: 29 (LCDR3 of Ab3)
QQYGSSPYT
SEQ ID NO: 30 (VH of Ab3)
QVQLQESGPGLVKPSETLSLTCTVSGGSISTYYWNWIRQPAGKGLEWIGRIYSSGS
TNYNPSLKSRVTMSVDTSKNQFSLKLSSVTAADTAVYYCARDPLYSGSLFDIWG
QGTMVTVSS
SEQ ID NO: 31 (VL of Ab3)
EIVLTQSPGTLSLSPGERATLSCRASQSVSSSYLAWYQQKPGQAPRLLIYGASIRA
TGIPDRFSGSGSGTDFTLTISRLEPEDFAVYYCQQYGSSPYTFGQGTKLEIK
SEQ ID NO: 32 (HC of Ab4)
EVQLVESGGGLVKPGGSLRLSCAASGFTFSSYSMNWVRQAPGKGLEWVSSIDSS
SSFIYYADSVKGRFTISRDNAKNSLYLQMNSLRAEDTAVYYCARGTGLFDFWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKS
CDKTHTCPPCPAPEAAGGPSVFLFPPKPKDTLMISRTPEVTCVVVSVSHEDPEVKF
NWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHY
TQKSLSLSPGK
SEQ ID NO: 33 (LC of Ab4)
EIVLTQSPGTLSLSPGERATLSCRASQSFSSSYLAWYQQKPGQAPRLLIYGASSRA
TGIPDRFSGSGSGTDFTLTISRLEPEDFAVYYCQQYGRTPWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 34 (HCDR1 of Ab4)
AASGFTFSSYSMN
SEQ ID NO: 35 (HCDR2 of Ab4)
SIDSSSSFIY
SEQ ID NO: 36 (HCDR3 of Ab4)
ARGTGLFDF
SEQ ID NO: 37 (LCDR1 of Ab4)
RASQSFSSSYLA
SEQ ID NO: 38 (LCDR3 of Ab4)
QQYGRTPWT
SEQ ID NO: 39 (VH of Ab4)
EVQLVESGGGLVKPGGSLRLSCAASGFTFSSYSMNWVRQAPGKGLEWVSSIDSS
SSFIYYADSVKGRFTISRDNAKNSLYLQMNSLRAEDTAVYYCARGTGLFDFWGQ
GTLVTVSS
SEQ ID NO: 40 (VL of Ab4)
EIVLTQSPGTLSLSPGERATLSCRASQSFSSSYLAWYQQKPGQAPRLLIYGASSRA
TGIPDRFSGSGSGTDFTLTISRLEPEDFAVYYCQQYGRTPWTFGQGTKVEIK
SEQ ID NO: 41 (DNA of HC for Ab1)
GAGGTGCAGCTGGTGGAGTCTGGGGGAGGCCTGGTCAAGCCTGGGGGGTCCC
TGAGACTCTCCTGTGCAGCCTCTGGATTCACCTTCAGTAGCTATATCATGAAC
TGGGTCCGCCAGGCTCCAGGGAAGGGACTGGAGTGGGTCTCTTCCATTAGTA
GTAGTAGTACTTTCATATACTACGCAGACTCAGTGAAGGGCCGATTCACCATC
TCCAGAGACAACGCCAAGAACTCACTGTATCTGCAAATGAACAGCCTGAGAG
CCGAGGACACGGCTGTGTATTACTGTGCGAAAGGTTTTGACTACTGGGGCCA
GGGAACCCTGGTCACCGTCTCCTCAgcctccaccaagggcccatcggtcttccccctggcaccctcctcc
aagagcacctctgggggcacagcggccctgggctgcctggtcaaggactacttccccgaaccggtgacggtgtcgtggaactc
aggcGCGctgaccagcggcgtgcacaccttcccggctgtcctacagtcctcaggactctactccctcagcagcgtggtgacc
gtgccctccagcagcttgggcacccagacctacatctgcaacgtgaatcacaagcccagcaacaccaaggtggacaagagag
ttgagcccaaatcttgtgacaaaactcacacatgcccaccgtgcccagcacctgaagccgccgggggaccgtcagtcttcctctt
ccccccaaaacccaaggacaccctcatgatctcccggacccctgaggtcacatgcgtggtggtgtccgtgagccacgaagacc
ctgaggtcaagttcaactggtatgtggacggcgtggaggtgcataatgccaagacaaagccgcgggaggagcagtacaacag
cacgtaccgtgtggtcagcgtcctcaccgtcctgcaccaagactggctgaatggcaaggagtacaagtgcaaggtctccaacaa
agccctcccagcccccatcgagaaaaccatctccaaagccaaagggcagccccgagaaccacaggtgtacaccctgccccca
tcccgggaggagatgaccaagaaccaagtcagcctgacctgcctggtcaaaggcttctatcccagcgacatcgccgtggagtg
ggagagcaatgggcagccggagaacaactacaagaccacgcctcccgtgctggactccgacggctccttcttcctctattccaa
gctcaccgtggacaagagcaggtggcagcaggggaacgtcttctcatgctccgtgatgcatgaggctctgcacaaccactaca
cgcagaagagcctctccctgtctccgggcaaatga
SEQ ID NO: 42 (DNA of LC for Ab1)
GACATCCAGTTGACCCAGTCTCCATCCTTCCTGTCTGCATCTGTAGGAGACAG
AGTCACCATCACTTGCCGGGCCAGTCAGGACATTAGCAGTTATTTAGTCTGGT
ATCAGCAAAAACCAGGGAAAGCCCCTAAGCTCCTGATCTATGCTGCATCCAC
TTTGCAAAGTGGGGTCCCATCAAGGTTCAGCGGCAGTGGATCTGGGACAGAA
TTCACTCTCACAATCAGCAGCCTGCAGCCTGAAGATTTTGCAACTTATTACTG
TCAACAACTTAATACTTACCCTCGGACGTTCGGCCAAGGGACCAAGGTGGAA
ATCAAAagaactgtggcggcgccatctgtcttcatcttcccgccatctgatgagcagttgaaatccggaactgcctctgttgt
gtgcctgctgaataacttctatcccagagaggccaaagtacagtggaaggtggataacgccctccaatcgggtaactcccagga
gagtgtcacagagcaggacagcaaggacagcacctacagcctcagcagcaccctgacgctgagcaaagcagactacgagaa
acacaaagtctacgcctgcgaagtcacccatcagggcctgagctcgcccgtcacaaagagcttcaacaggggagagtgctaa
SEQ ID NO: 43 (DNA of HC for Ab2)
CAGGTGCAGCTGCAGGAGTCGGGCCCAGGACTGGTGAAGCCTTCGGAGACCC
TGTCCCTCACCTGCACTGTCTCTGGTGGCTCCATCAGTACTTACTACTGGAGC
TGGATCCGGCAGCCCGCCGGGAAGGGACTGGAGTGGATTGGACGTATCTATA
CCAGTGGTCTCACCAACTACAACCCCTCCCTCAAGAGTCGAGTCACCATGTCT
GTAGACACGTCCAAGAACCAGTTCTCCCTGAAGCTGAGCTCTGTGACCGCCG
CGGACACGGCCGTATATTACTGTGCGAGAGACCCCCATTACTATGATGGTAG
TGGTTTTGACTACTGGGGCCAGGGAACCCTGGTCACCGTCTCCTCAgcctccaccaa
gggcccatcggtcttccccctggcaccctcctccaagagcacctctgggggcacagcggccctgggctgcctggtcaaggact
acttccccgaaccggtgacggtgtcgtggaactcaggcGCGctgaccagcggcgtgcacaccttcccggctgtcctacagtc
ctcaggactctactccctcagcagcgtggtgaccgtgccctccagcagcttgggcacccagacctacatctgcaacgtgaatca
caagcccagcaacaccaaggtggacaagagagttgagcccaaatcttgtgacaaaactcacacatgcccaccgtgcccagca
cctgaagccgccgggggaccgtcagtcttcctcttccccccaaaacccaaggacaccctcatgatctcccggacccctgaggtc
acatgcgtggtggtgtccgtgagccacgaagaccctgaggtcaagttcaactggtatgtggacggcgtggaggtgcataatgcc
aagacaaagccgcgggaggagcagtacaacagcacgtaccgtgtggtcagcgtcctcaccgtcctgcaccaagactggctga
atggcaaggagtacaagtgcaaggtctccaacaaagccctcccagcccccatcgagaaaaccatctccaaagccaaagggca
gccccgagaaccacaggtgtacaccctgcccccatcccgggaggagatgaccaagaaccaagtcagcctgacctgcctggtc
aaaggcttctatcccagcgacatcgccgtggagtgggagagcaatgggcagccggagaacaactacaagaccacgcctcccg
tgctggactccgacggctccttcttcctctattccaagctcaccgtggacaagagcaggtggcagcaggggaacgtcttctcatg
ctccgtgatgcatgaggctctgcacaaccactacacgcagaagagcctctccctgtctccgggcaaatga
SEQ ID NO: 44 (DNA of LC for Ab2)
GAAATTGTGTTGACGCAGTCTCCAGGCACCCTGTCTTTGTCTCCAGGGGAAAG
AGCCACCCTCTCCTGCAGGGCCAGTCAGAGTGTTAGCGACAACTACTTAGCCT
GGTACCAGCAGAAACCTGGCCAGGCTCCCAGGCTCCTCATCTATGGTGCATC
CAGCAGGGCCACTGGCATCCCAGACAGGTTCAGTGGCAGTGGGTCTGGGACA
GACTTCACTCTCACCATCAGCAGACTGGAGCCTGAAGATTTTGCAGTGTACTA
CTGTCAGCAGTATGGTAGTTCACCATTCACTTTCGGCCCTGGGACCAAGGTGG
ATATCAAAagaactgtggcggcgccatctgtcttcatcttcccgccatctgatgagcagttgaaatccggaactgcctctgt
tgtgtgcctgctgaataacttctatcccagagaggccaaagtacagtggaaggtggataacgccctccaatcgggtaactcccag
gagagtgtcacagagcaggacagcaaggacagcacctacagcctcagcagcaccctgacgctgagcaaagcagactacgag
aaacacaaagtctacgcctgcgaagtcacccatcagggcctgagctcgcccgtcacaaagagcttcaacaggggagagtgcta
a
SEQ ID NO: 45 (DNA of HC for Ab3)
CAGGTGCAGCTGCAGGAGTCGGGCCCAGGACTGGTGAAGCCTTCGGAGACCC
TGTCCCTCACCTGCACTGTCTCTGGTGGCTCCATCAGTACTTACTACTGGAAC
TGGATCCGGCAGCCCGCCGGGAAGGGACTGGAGTGGATTGGGCGTATCTATA
GTAGTGGGAGCACCAACTACAACCCCTCCCTCAAGAGTCGAGTCACCATGTC
AGTAGACACGTCCAAGAACCAGTTCTCCCTGAAGCTGAGCTCTGTGACCGCC
GCGGACACGGCCGTGTATTACTGTGCGAGAGATCCCTTGTATAGTGGGAGCC
TTTTTGATATCTGGGGCCAAGGGACAATGGTCACCGTCTCTTCAgcctccaccaaggg
cccatcggtcttccccctggcaccctcctccaagagcacctctgggggcacagcggccctgggctgcctggtcaaggactactt
ccccgaaccggtgacggtgtcgtggaactcaggcGCGctgaccagcggcgtgcacaccttcccggctgtcctacagtcctca
ggactctactccctcagcagcgtggtgaccgtgccctccagcagcttgggcacccagacctacatctgcaacgtgaatcacaag
cccagcaacaccaaggtggacaagagagttgagcccaaatcttgtgacaaaactcacacatgcccaccgtgcccagcacctga
agccgccgggggaccgtcagtcttcctcttccccccaaaacccaaggacaccctcatgatctcccggacccctgaggtcacatg
cgtggtggtgtccgtgagccacgaagaccctgaggtcaagttcaactggtatgtggacggcgtggaggtgcataatgccaaga
caaagccgcgggaggagcagtacaacagcacgtaccgtgtggtcagcgtcctcaccgtcctgcaccaagactggctgaatgg
caaggagtacaagtgcaaggtctccaacaaagccctcccagcccccatcgagaaaaccatctccaaagccaaagggcagccc
cgagaaccacaggtgtacaccctgcccccatcccgggaggagatgaccaagaaccaagtcagcctgacctgcctggtcaaag
gcttctatcccagcgacatcgccgtggagtgggagagcaatgggcagccggagaacaactacaagaccacgcctcccgtgct
ggactccgacggctccttcttcctctattccaagctcaccgtggacaagagcaggtggcagcaggggaacgtcttctcatgctcc
gtgatgcatgaggctctgcacaaccactacacgcagaagagcctctccctgtctccgggcaaatga
SEQ ID NO: 46 (DNA of LC for Ab3)
GAAATTGTGTTGACGCAGTCTCCAGGCACCCTGTCTTTGTCTCCAGGGGAAAG
AGCCACCCTCTCCTGCAGGGCCAGTCAGAGTGTTAGCAGCAGCTACTTAGCCT
GGTACCAACAGAAACCTGGCCAGGCTCCCAGGCTCCTCATCTATGGTGCATC
CATCAGGGCCACTGGCATCCCAGACAGGTTCAGTGGCAGTGGGTCTGGGACA
GACTTCACTCTCACCATCAGCAGACTGGAGCCTGAAGATTTTGCAGTGTATTA
CTGTCAGCAGTATGGTAGCTCACCGTACACTTTTGGCCAGGGGACCAAGCTG
GAGATCAAAagaactgtggcggcgccatctgtcttcatcttcccgccatctgatgagcagttgaaatccggaactgcctct
gttgtgtgcctgctgaataacttctatcccagagaggccaaagtacagtggaaggtggataacgccctccaatcgggtaactccc
aggagagtgtcacagagcaggacagcaaggacagcacctacagcctcagcagcaccctgacgctgagcaaagcagactacg
agaaacacaaagtctacgcctgcgaagtcacccatcagggcctgagctcgcccgtcacaaagagcttcaacaggggagagtg
ctaa
SEQ ID NO: 47 (DNA of HC for Ab4)
GAGGTGCAGCTGGTGGAGTCTGGGGGAGGCCTGGTCAAGCCTGGGGGGTCCC
TGAGACTCTCCTGTGCAGCCTCTGGATTCACCTTCAGTAGCTATAGCATGAAC
TGGGTCCGCCAGGCTCCAGGGAAGGGGCTGGAGTGGGTCTCATCCATTGATA
GTAGTAGTAGTTTCATATACTACGCAGACTCAGTGAAGGGCCGATTCACCATC
TCCAGGGACAACGCCAAGAACTCACTGTATCTGCAAATGAACAGCCTGAGAG
CCGAGGACACGGCCGTGTATTATTGTGCGAGAGGAACTGGGCTCTTTGACTTC
TGGGGCCAGGGAACCCTGGTCACCGTCTCCTCAgcctccaccaagggcccatcggtcttccccct
ggcaccctcctccaagagcacctctgggggcacagcggccctgggctgcctggtcaaggactacttccccgaaccggtgacg
gtgtcgtggaactcaggcGCGctgaccagcggcgtgcacaccttcccggctgtcctacagtcctcaggactctactccctcag
cagcgtggtgaccgtgccctccagcagcttgggcacccagacctacatctgcaacgtgaatcacaagcccagcaacaccaag
gtggacaagagagttgagcccaaatcttgtgacaaaactcacacatgcccaccgtgcccagcacctgaagccgccgggggac
cgtcagtcttcctcttccccccaaaacccaaggacaccctcatgatctcccggacccctgaggtcacatgcgtggtggtgtccgtg
agccacgaagaccctgaggtcaagttcaactggtatgtggacggcgtggaggtgcataatgccaagacaaagccgcgggagg
agcagtacaacagcacgtaccgtgtggtcagcgtcctcaccgtcctgcaccaagactggctgaatggcaaggagtacaagtgc
aaggtctccaacaaagccctcccagcccccatcgagaaaaccatctccaaagccaaagggcagccccgagaaccacaggtgt
acaccctgcccccatcccgggaggagatgaccaagaaccaagtcagcctgacctgcctggtcaaaggcttctatcccagcgac
atcgccgtggagtgggagagcaatgggcagccggagaacaactacaagaccacgcctcccgtgctggactccgacggctcct
tcttcctctattccaagctcaccgtggacaagagcaggtggcagcaggggaacgtcttctcatgctccgtgatgcatgaggctctg
cacaaccactacacgcagaagagcctctccctgtctccgggcaaatga
SEQ ID NO: 48 (DNA of LC for Ab4)
GAAATTGTGTTGACGCAGTCTCCAGGCACCCTGTCTTTGTCTCCAGGGGAAAG
AGCCACCCTCTCCTGCAGGGCCAGTCAGAGTTTTAGCAGCAGCTACTTAGCCT
GGTACCAGCAGAAACCTGGCCAGGCTCCCAGGCTCCTCATCTATGGTGCATC
CAGCAGGGCCACTGGCATCCCAGACAGGTTCAGTGGCAGTGGGTCTGGGACA
GACTTCACTCTCACCATCAGCAGACTGGAGCCTGAAGATTTTGCAGTGTATTA
CTGTCAGCAGTATGGTAGGACACCGTGGACGTTCGGCCAAGGGACCAAGGTG
GAAATCAAAagaactgtggcggcgccatctgtcttcatcttcccgccatctgatgagcagttgaaatccggaactgcctct
gttgtgtgcctgctgaataacttctatcccagagaggccaaagtacagtggaaggtggataacgccctccaatcgggtaactccc
aggagagtgtcacagagcaggacagcaaggacagcacctacagcctcagcagcaccctgacgctgagcaaagcagactacg
agaaacacaaagtctacgcctgcgaagtcacccatcagggcctgagctcgcccgtcacaaagagcttcaacaggggagagtg
ctaa
SEQ ID NO: 49:
-Ala-Leu-Ala-Leu-(also known as ALAL in one letter code)
SEQ ID NO: 50:
-Leu-Ala-Leu-Ala-(also known as LALA in one letter code)
SEQ ID NO: 51
-Gly-Phe-Leu-Gly-(also known as GFLG in one letter code)
SEQ ID NO: 52
-Gly-Leu-Phe-Gly-(also known as GLFG in one letter code)
SEQ ID NO: 53
-Ala-Ala-Ala-Ala-(also known as AAAA in one letter code)
SEQ ID NO: 54
-Gly-Ala-Gly-Gly-(also known as GAGG in one letter code)
SEQ ID NO: 55
-Gly-Gly-Ala-Gly-(also known as GGAG in one letter code)
SEQ ID NO: 56
-Gly-Val-Gly-Gly-(also known as GVGG in one letter code)
SEQ ID NO: 57
-Gly-Gly-Val-Gly-(also known as GGVG in one letter code)
SEQ ID NO: 58:
-Gly-Phe-Gly-Gly-(also known as GFGG in one letter code)
SEQ ID NO: 59
-Gly-Gly-Phe-Gly-(also known as GGFG in one letter code)

We claim:

1. An antibody-drug conjugate (ADC) comprising an antibody conjugated to a cytotoxic agent, wherein the antibody binds human PTK-7, and comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein:
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19;
c) the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29; or
d) the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

2. The ADC of claim 1, wherein:
a) the VH comprises SEQ ID NO: 10 and the VL comprises SEQ ID NO: 11;
b) the VH comprises SEQ ID NO: 20 and the VL comprises SEQ ID NO: 21;
c) the VH comprises SEQ ID NO: 30 and the VL comprises SEQ ID NO: 31; or
d) the VH comprises SEQ ID NO: 39 and the VL comprises SEQ ID NO: 40.

3. The ADC of claim 1, wherein the antibody comprises a heavy chain (HC) and a light chain (LC), wherein:
a) the HC comprises amino acids 2-441 of SEQ ID NO: 2 and the LC comprises SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23; or
d) the HC comprises amino acids 2-444 of SEQ ID NO: 32 and the LC comprises amino acids 2-215 of SEQ ID NO: 33.

4. The ADC of claim 3, wherein:
a) the HC consists of SEQ ID NO: 2 and the LC consists of SEQ ID NO: 3;
b) the HC consists of SEQ ID NO: 12 and the LC consists of SEQ ID NO: 13;
c) the HC consists of SEQ ID NO: 22 and the LC consists of SEQ ID NO: 23; or
d) the HC consists of SEQ ID NO: 32 and the LC consists of SEQ ID NO: 33.

5. The ADC of claim 1, wherein the cytotoxic agent is selected from the group consisting of a microtubule inhibitor, a topoisomerase I inhibitor, a DNA damaging agent, a DNA alkylating agent, and a DNA minor groove binder.

6. The ADC of claim 5, wherein the cytotoxic agent is a topoisomerase I inhibitor.

7. The ADC of claim 6, wherein the topoisomerase I inhibitor is a camptothecin analog.

8. The ADC of claim 7, wherein the camptothecin analog comprises the Formula:

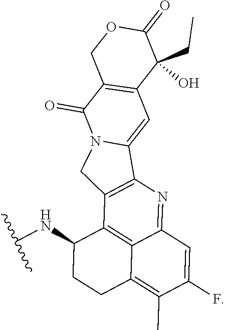 [SMILES: *N[C@@H]1CCc2c(C)c(F)cc3nc4c(c1c23)Cn1c-4cc2c(c1=O)COC(=O)[C@@]2(O)CC]

9. The ADC of claim 8, wherein the ADC comprises the Formula:

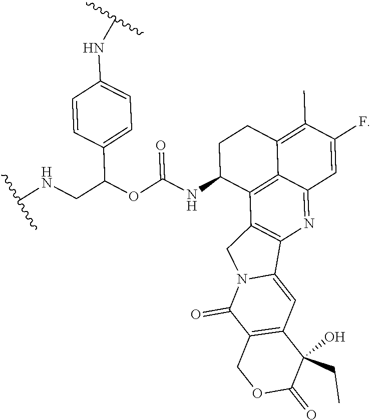 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(N*)cc1]

10. The ADC of claim 9, wherein the ADC further comprises a linker which connects the antibody to the cytotoxic agent.

11. The ADC of claim 10, wherein the linker comprises a peptide unit or a sugar cleavable unit.

12. The ADC of claim 11, wherein the peptide unit comprises Val-Ala, Val-Cit, Phe-Lys, or Ala-Ala-Asn.

13. The ADC of claim 12, wherein the ADC comprises the Formula:

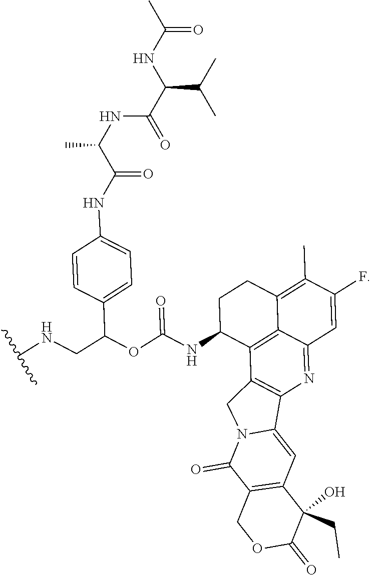 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(NC(=O)[C@H](C)NC(=O)[C@@H](NC(C)=O)C(C)C)cc1]

14. The ADC of claim 11, wherein the sugar cleavable unit comprises the Formula:

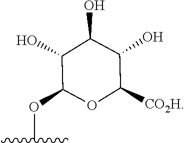 [SMILES: *O[C@@H]1O[C@H](C(=O)O)[C@@H](O)[C@H](O)[C@H]1O]

15. The ADC of claim 14, wherein the ADC comprises the Formula:

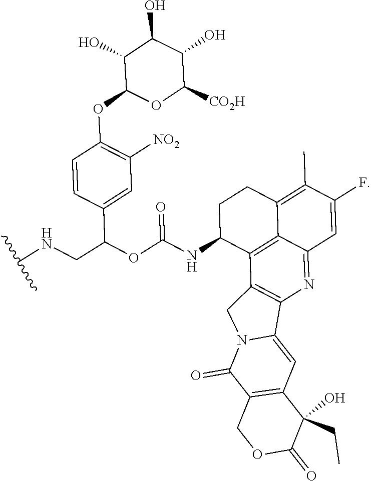 [SMILES: *NCC(OC(=O)N[C@H]1CCc2c(C)c(F)cc3nc4c(c1c23)Cn1c-4cc2c(c1=O)COC(=O)[C@]2(O)CC)c1ccc(O[C@@H]2O[C@H](C(=O)O)[C@@H](O)[C@H](O)[C@H]2O)c([N+](=O)[O-])c1]

16. The ADC of claim 11, wherein the linker further comprises a hydrophobicity masking group.

17. The ADC of claim 16, wherein the hydrophobicity masking group is selected from polysarcosine or polyethylene glycol.

18. The ADC of claim 17, wherein the hydrophobicity masking group is in a branched configuration on the linker.

19. The ADC of claim 18, wherein the hydrophobicity masking group is polysarcosine of the Formula: 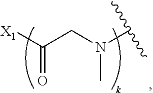
and wherein k is an integer from 6 to 12, and X₁ is H, OH, or NH₂.

20. The ADC of claim 16, wherein the linker further comprises a connecting unit.

21. The ADC of claim 20, wherein the connecting unit is one of the Formula: 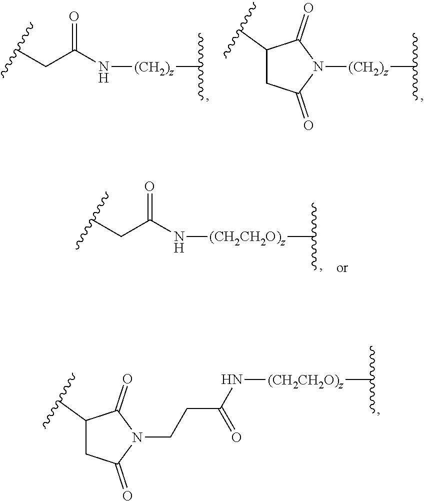
and
wherein z is from 1 to 5.

22. The ADC of claim 3, wherein the ADC is one of the Formula: 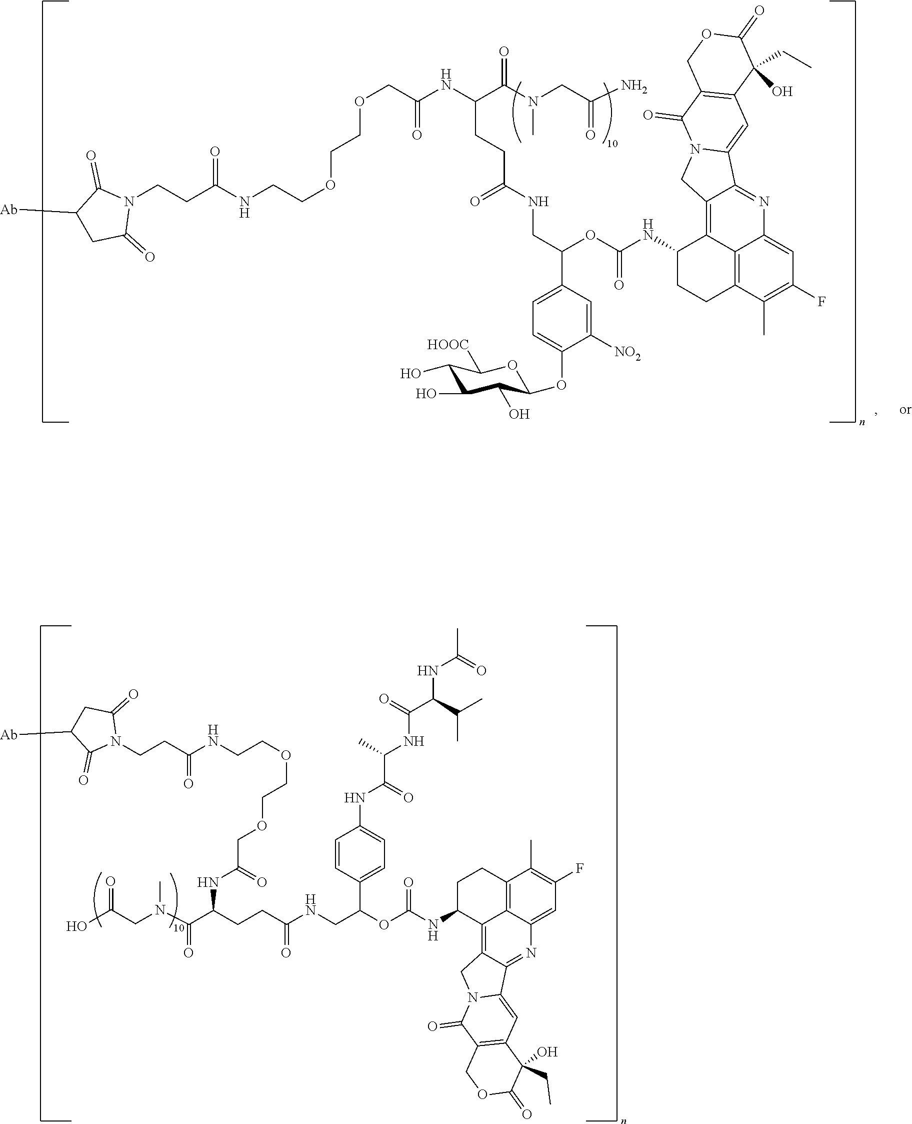
wherein:
Ab is the antibody and wherein:
a) the HC comprises amino acids 2-441 of SEQ ID NO: 2 and the LC comprises SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23; or
d) the HC comprises amino acids 2-444 of SEQ ID NO: 32 and the LC comprises amino acids 2-215 of SEQ ID NO: 33, and
n is from about 1 to 16.

23. The ADC of claim 22, wherein the ADC is of the Formula:

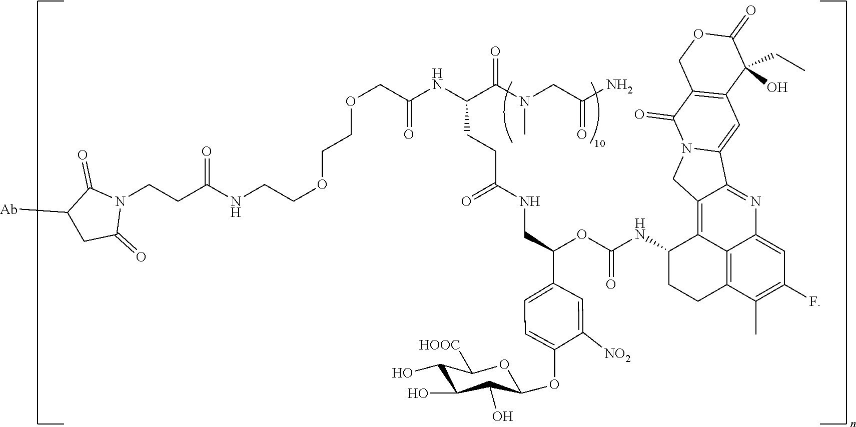

24. The ADC of claim 22, wherein the ADC is of the Formula:

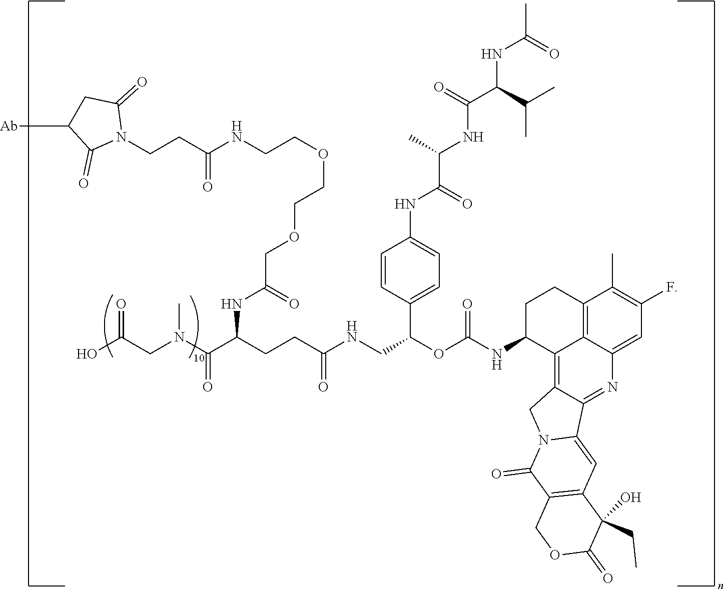

25. The ADC of claim 22, wherein connection to the antibody occurs through a thiol group on one or more cysteines of the antibody.

26. The ADC of claim 25, wherein the one or more cysteines are each a natural cysteine in the hinge region of the antibody.

27. The ADC of claim 26, wherein the Ab comprises a HC comprising amino acids 2-441 of SEQ ID NO: 2 and a LC comprising SEQ ID NO: 3, and wherein n is about 8.

28. The ADC of claim 26, wherein the Ab comprises a HC comprising amino acids 2-448 of SEQ ID NO: 12 and a LC comprising amino acids 2-215 of SEQ ID NO: 13, and wherein n is about 8.

29. The ADC of claim 26, wherein the Ab comprises a HC comprising amino acids 2-447 of SEQ ID NO: 22 and a LC comprising amino acids 2-215 of SEQ ID NO: 23, and wherein n is about 8.

30. The ADC of claim 26, wherein the Ab comprising amino acids 2-444 of SEQ ID NO: 32 and a LC comprising amino acids 2-215 of SEQ ID NO: 33, and wherein n is about 8.

31. An antibody that binds human PTK-7, wherein the antibody comprises a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3, wherein:
a) the HCDR1 comprises SEQ ID NO: 4, the HCDR2 comprises SEQ ID NO: 5, the HCDR3 comprises SEQ ID NO: 6, the LCDR1 comprises SEQ ID NO: 7, the LCDR2 comprises SEQ ID NO: 8, and the LCDR3 comprises SEQ ID NO: 9;
b) the HCDR1 comprises SEQ ID NO: 14, the HCDR2 comprises SEQ ID NO: 15, the HCDR3 comprises SEQ ID NO: 16, the LCDR1 comprises SEQ ID NO: 17, the LCDR2 comprises SEQ ID NO: 18, and the LCDR3 comprises SEQ ID NO: 19;
c) the HCDR1 comprises SEQ ID NO: 24, the HCDR2 comprises SEQ ID NO: 25, the HCDR3 comprises SEQ ID NO: 26, the LCDR1 comprises SEQ ID NO: 27, the LCDR2 comprises SEQ ID NO: 28, and the LCDR3 comprises SEQ ID NO: 29; or
d) the HCDR1 comprises SEQ ID NO: 34, the HCDR2 comprises SEQ ID NO: 35, the HCDR3 comprises SEQ ID NO: 36, the LCDR1 comprises SEQ ID NO: 37, the LCDR2 comprises SEQ ID NO: 18, and the LCDR3 comprises SEQ ID NO: 38.

32. The antibody of claim 31, wherein:
a) the VH comprises SEQ ID NO: 10 and the VL comprises SEQ ID NO: 11;
b) the VH comprises SEQ ID NO: 20 and the VL comprises SEQ ID NO: 21;
c) the VH comprises SEQ ID NO: 30 and the VL comprises SEQ ID NO: 31; or
d) the VH comprises SEQ ID NO: 39 and the VL comprises SEQ ID NO: 40.

33. The antibody of claim 31, wherein the antibody comprises a heavy chain (HC) and a light chain (LC), wherein:
a) the HC comprises amino acids 2-441 of SEQ ID NO: 2 and the LC comprises SEQ ID NO: 3;
b) the HC comprises amino acids 2-448 of SEQ ID NO: 12 and the LC comprises amino acids 2-215 of SEQ ID NO: 13;
c) the HC comprises amino acids 2-447 of SEQ ID NO: 22 and the LC comprises amino acids 2-215 of SEQ ID NO: 23; or
d) the HC comprises amino acids 2-444 of SEQ ID NO: 32 and the LC comprises amino acids 2-215 of SEQ ID NO: 33.

34. The antibody of claim 33, wherein:
a) the HC consists of SEQ ID NO: 2 and the LC consists of SEQ ID NO: 3;
b) the HC consists of SEQ ID NO: 12 and the LC consists of SEQ ID NO: 13;
c) the HC consists of SEQ ID NO: 22 and the LC consists of SEQ ID NO: 23; or
d) the HC consists of SEQ ID NO: 32 and the LC consists of SEQ ID NO: 33.

35. A pharmaceutical composition comprising the ADC of claim 1 and one or more pharmaceutically acceptable carriers, diluents, or excipients.

36. A method of treating cancer, comprising administering to a patient in need thereof, an effective amount of the ADC of claim 1.

37. The method of claim 36, wherein the cancer is ovarian cancer, lung cancer, breast cancer, gastric cancer, kidney cancer, prostate cancer, liver cancer, or colorectal cancer.

38. The method of claim 37, further comprising administrating simultaneously, separately, or sequentially a PD-1 inhibitor or PD-L1 inhibitor.

39. A method of preparing an ADC comprising conjugating the antibody of claim 31 to a linker-payload, wherein the linker-payload comprises one of the Formula:

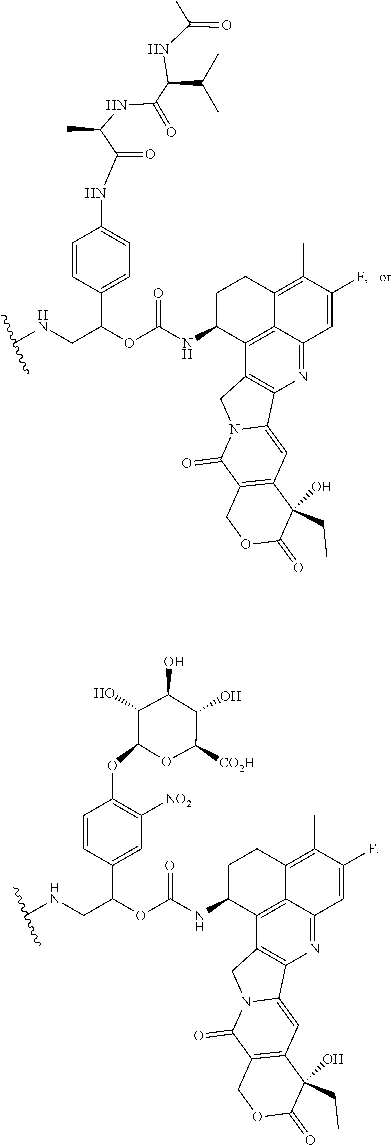

40. A method of producing an ADC, the method comprising contacting the antibody of claim 31 with a compound of the Formula: [chemical structure]


SEQUENCE LISTING (59 sequences)

SEQ ID NO: 1 - protein, 1070 aa, Homo sapiens
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 2 - protein, 443 aa, synthetic construct
EVQLVESGGGLVKPGGSLRLSCAASGFTFSSYIMNWVRQAPGKGLEWVSSISSSSTFIYYADSVKGRFTISRDNAKNSLYLQMNSLRAEDTAVYYCAKGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIQLTQSPSFLSASVGDRVTITCRASQDISSYLVWYQQKPGKAPKLLIYAASTLQSGVPSRFSGSGSGTEFTLTISSLQPEDFATYYCQQLNTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 13 aa, synthetic construct
AASGFTFSSYIMN

SEQ ID NO: 5 - protein, 10 aa, synthetic construct
SISSSSTFIY

SEQ ID NO: 6 - protein, 6 aa, synthetic construct
AKGFDY

SEQ ID NO: 7 - protein, 11 aa, synthetic construct
RASQDISSYLV

SEQ ID NO: 8 - protein, 8 aa, synthetic construct
YAASTLQS

SEQ ID NO: 9 - protein, 9 aa, synthetic construct
QQLNTYPRT

SEQ ID NO: 10 - protein, 113 aa, synthetic construct
EVQLVESGGGLVKPGGSLRLSCAASGFTFSSYIMNWVRQAPGKGLEWVSSISSSSTFIYYADSVKGRFTISRDNAKNSLYLQMNSLRAEDTAVYYCAKGFDYWGQGTLVTVSS

SEQ ID NO: 11 - protein, 107 aa, synthetic construct
DIQLTQSPSFLSASVGDRVTITCRASQDISSYLVWYQQKPGKAPKLLIYAASTLQSGVPSRFSGSGSGTEFTLTISSLQPEDFATYYCQQLNTYPRTFGQGTKVEIK

SEQ ID NO: 12 - protein, 450 aa, synthetic construct
QVQLQESGPGLVKPSETLSLTCTVSGGSISTYYWSWIRQPAGKGLEWIGRIYTSGLTNYNPSLKSRVTMSVDTSKNQFSLKLSSVTAADTAVYYCARDPHYYDGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 215 aa, synthetic construct
EIVLTQSPGTLSLSPGERATLSCRASQSVSDNYLAWYQQKPGQAPRLLIYGASSRATGIPDRFSGSGSGTDFTLTISRLEPEDFAVYYCQQYGSSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 13 aa, synthetic construct
TVSGGSISTYYWS

SEQ ID NO: 15 - protein, 9 aa, synthetic construct
RIYTSGLTN

SEQ ID NO: 16 - protein, 14 aa, synthetic construct
ARDPHYYDGSGFDY

SEQ ID NO: 17 - protein, 12 aa, synthetic construct
RASQSVSDNYLA

SEQ ID NO: 18 - protein, 8 aa, synthetic construct
YGASSRAT

SEQ ID NO: 19 - protein, 9 aa, synthetic construct
QQYGSSPFT

SEQ ID NO: 20 - protein, 120 aa, synthetic construct
QVQLQESGPGLVKPSETLSLTCTVSGGSISTYYWSWIRQPAGKGLEWIGRIYTSGLTNYNPSLKSRVTMSVDTSKNQFSLKLSSVTAADTAVYYCARDPHYYDGSGFDYWGQGTLVTVSS

SEQ ID NO: 21 - protein, 108 aa, synthetic construct
EIVLTQSPGTLSLSPGERATLSCRASQSVSDNYLAWYQQKPGQAPRLLIYGASSRATGIPDRFSGSGSGTDFTLTISRLEPEDFAVYYCQQYGSSPFTFGPGTKVDIK

SEQ ID NO: 22 - protein, 449 aa, synthetic construct
QVQLQESGPGLVKPSETLSLTCTVSGGSISTYYWNWIRQPAGKGLEWIGRIYSSGSTNYNPSLKSRVTMSVDTSKNQFSLKLSSVTAADTAVYYCARDPLYSGSLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 215 aa, synthetic construct
EIVLTQSPGTLSLSPGERATLSCRASQSVSSSYLAWYQQKPGQAPRLLIYGASIRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 13 aa, synthetic construct
TVSGGSISTYYWN

SEQ ID NO: 25 - protein, 9 aa, synthetic construct
RIYSSGSTN

SEQ ID NO: 26 - protein, 13 aa, synthetic construct
ARDPLYSGSLFDI

SEQ ID NO: 27 - protein, 12 aa, synthetic construct
RASQSVSSSYLA

SEQ ID NO: 28 - protein, 8 aa, synthetic construct
YGASIRAT

SEQ ID NO: 29 - protein, 9 aa, synthetic construct
QQYGSSPYT

SEQ ID NO: 30 - protein, 119 aa, synthetic construct
QVQLQESGPGLVKPSETLSLTCTVSGGSISTYYWNWIRQPAGKGLEWIGRIYSSGSTNYNPSLKSRVTMSVDTSKNQFSLKLSSVTAADTAVYYCARDPLYSGSLFDIWGQGTMVTVSS

SEQ ID NO: 31 - protein, 108 aa, synthetic construct
EIVLTQSPGTLSLSPGERATLSCRASQSVSSSYLAWYQQKPGQAPRLLIYGASIRATGIPDRFSGSGSGTDFTLTISRLEPEDFAVYYCQQYGSSPYTFGQGTKLEIK

SEQ ID NO: 32 - protein, 446 aa, synthetic construct
EVQLVESGGGLVKPGGSLRLSCAASGFTFSSYSMNWVRQAPGKGLEWVSSIDSSSSFIYYADSVKGRFTISRDNAKNSLYLQMNSLRAEDTAVYYCARGTGLFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 215 aa, synthetic construct
EIVLTQSPGTLSLSPGERATLSCRASQSFSSSYLAWYQQKPGQAPRLLIYGASSRATGIPDRFSGSGSGTDFTLTISRLEPEDFAVYYCQQYGRTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 13 aa, synthetic construct
AASGFTFSSYSMN

SEQ ID NO: 35 - protein, 10 aa, synthetic construct
SIDSSSSFIY

SEQ ID NO: 36 - protein, 9 aa, synthetic construct
ARGTGLFDF

SEQ ID NO: 37 - protein, 12 aa, synthetic construct
RASQSFSSSYLA

SEQ ID NO: 38 - protein, 9 aa, synthetic construct
QQYGRTPWT

SEQ ID NO: 39 - protein, 116 aa, synthetic construct
EVQLVESGGGLVKPGGSLRLSCAASGFTFSSYSMNWVRQAPGKGLEWVSSIDSSSSFIYYADSVKGRFTISRDNAKNSLYLQMNSLRAEDTAVYYCARGTGLFDFWGQGTLVTVSS

SEQ ID NO: 40 - protein, 108 aa, synthetic construct
EIVLTQSPGTLSLSPGERATLSCRASQSFSSSYLAWYQQKPGQAPRLLIYGASSRATGIPDRFSGSGSGTDFTLTISRLEPEDFAVYYCQQYGRTPWTFGQGTKVEIK

SEQ ID NO: 41 - DNA, 1332 bp, synthetic construct
  source 1..1332: mol_type = other DNA
gaggtgcagctggtggagtctgggggaggcctggtcaagcctggggggtccctgagactctcctgtgcagcctctggattcaccttcagtagctatatcatgaactgggtccgccaggctccagggaagggactggagtgggtctcttccattagtagtagtagtactttcatatactacgcagactcagtgaagggccgattcaccatctccagagacaacgccaagaactcactgtatctgcaaatgaacagcctgagagccgaggacacggctgtgtattactgtgcgaaaggttttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 42 - DNA, 645 bp, synthetic construct
  source 1..645: mol_type = other DNA
gacatccagttgacccagtctccatccttcctgtctgcatctgtaggagacagagtcaccatcacttgccgggccagtcaggacattagcagttatttagtctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacaacttaatacttaccctcggacgttcggccaagggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaa

SEQ ID NO: 43 - DNA, 1353 bp, synthetic construct
  source 1..1353: mol_type = other DNA
caggtgcagctgcaggagtcgggcccaggactggtgaagccttcggagaccctgtccctcacctgcactgtctctggtggctccatcagtacttactactggagctggatccggcagcccgccgggaagggactggagtggattggacgtatctataccagtggtctcaccaactacaacccctccctcaagagtcgagtcaccatgtctgtagacacgtccaagaaccagttctccctgaagctgagctctgtgaccgccgcggacacggccgtatattactgtgcgagagacccccattactatgatggtagtggttttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 44 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagtgttagcgacaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtactactgtcagcagtatggtagttcaccattcactttcggccctgggaccaaggtggatatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaa

SEQ ID NO: 45 - DNA, 1350 bp, synthetic construct
  source 1..1350: mol_type = other DNA
caggtgcagctgcaggagtcgggcccaggactggtgaagccttcggagaccctgtccctcacctgcactgtctctggtggctccatcagtacttactactggaactggatccggcagcccgccgggaagggactggagtggattgggcgtatctatagtagtgggagcaccaactacaacccctccctcaagagtcgagtcaccatgtcagtagacacgtccaagaaccagttctccctgaagctgagctctgtgaccgccgcggacacggccgtgtattactgtgcgagagatcccttgtatagtgggagcctttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 46 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccatcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaa

SEQ ID NO: 47 - DNA, 1341 bp, synthetic construct
  source 1..1341: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattgatagtagtagtagtttcatatactacgcagactcagtgaagggccgattcaccatctccagggacaacgccaagaactcactgtatctgcaaatgaacagcctgagagccgaggacacggccgtgtattattgtgcgagaggaactgggctctttgacttctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 48 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagttttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtaggacaccgtggacgttcggccaagggaccaaggtggaaatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaa

SEQ ID NO: 49 - protein, 4 aa, synthetic construct
ALAL

SEQ ID NO: 50 - protein, 4 aa, synthetic construct
LALA

SEQ ID NO: 51 - protein, 4 aa, synthetic construct
GFLG

SEQ ID NO: 52 - protein, 4 aa, synthetic construct
GLFG

SEQ ID NO: 53 - protein, 4 aa, synthetic construct
AAAA

SEQ ID NO: 54 - protein, 4 aa, synthetic construct
GAGG

SEQ ID NO: 55 - protein, 4 aa, synthetic construct
GGAG

SEQ ID NO: 56 - protein, 4 aa, synthetic construct
GVGG

SEQ ID NO: 57 - protein, 4 aa, synthetic construct
GGVG

SEQ ID NO: 58 - protein, 4 aa, synthetic construct
GFGG

SEQ ID NO: 59 - protein, 4 aa, synthetic construct
GGFG